(19) United States
(12) Patent Application Publication
(10) Pub. No.: US 2010/0099093 A1
(43) Pub. Date: Apr. 22, 2010

(54) Biomarkers for the Identification Monitoring and Treatment of Head and Neck Cancer

(75) Inventors: David Weaver, Newton, MA (US); XioaZhe Wang, Auburdale, MA (US); Kam Marie Sprott, Needham, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: THE DNA REPAIR COMPANY, INC., Cambridge, MA (US)

(21) Appl. No.: 12/466,291

(22) Filed: May 14, 2009

Related U.S. Application Data
(60) Provisional application No. 61/053,210, filed on May 14, 2008.

(51) Int. Cl. C12Q 1/68

(52) U.S. Cl. 435/6

(57) ABSTRACT

This present invention compositions and methods of treating cancer and methods of accessing/monitoring the responsiveness of a cancer cell to a therapeutic compound.

### RELATED APPLICATIONS

This application claims the benefit of U.S. Ser. No. 61/053,210 filed May 14, 2008 the contents of which are incorporated by reference in its entirety.

### FIELD OF THE INVENTION

The present invention relates generally to the identification of biomarkers and methods of using such biomarkers in the screening, prevention, diagnosis, therapy, monitoring, and prognosis of Head and Neck cancer.

### BACKGROUND OF THE INVENTION

Head and neck cancer represents the fifth most common malignancy worldwide, it is the most common neoplasm in the upper aerodigestive tract (Parkin D M et al. 2001). The majority of HN malignancies are squamous cell carcinomas (SCC). For practical purposes, head and neck cancer is divided into three clinical stages: early, locoregionally advanced, and metastatic or recurrent. Treatment approaches can vary depending on the disease stage. Chemotherapy in the treatment of locoregionally advanced head and neck cancer has improved disease-free and/or overall survival outcome, and concurrent chemoradiotherapy has been accepted as a standard treatment for patients with locoregionally advanced unresectable disease (Seiwert T Y et al 2007; Salama J K et al 2007). Current therapeutic decisions, which, however, often fail to predict patient outcome. Relatively little biomarker information is available for head and neck cancer patients stratification and to direct treatment decisions. Therefore, better methods of cancer detection, methods of molecular margin evaluation and prognostic biomarkers are of significant utility. This should lead to an improved stratification between higher-risk and lower-risk patients, which can be treated in a more selective and personalized manner.

DNA repair refers to a collection of processes by which a cell identifies and corrects damage to the DNA molecules that encode its genome. In human cells, both normal metabolic activities and environmental factors such as UV light can cause DNA damage, resulting in as many as 1 million individual molecular lesions per cell per day. Many of these lesions cause structural damage to the DNA molecule and can alter or eliminate the cell's ability to transcribe the gene that the affected DNA encodes. Other lesions induce potentially harmful mutations in the cell's genome, which will affect the survival of its daughter cells after it undergoes mitosis. Consequently, the DNA repair process must be constantly active so it can respond rapidly to any damage in the DNA structure.

The rate of DNA repair is dependent on many factors, including the cell type, the age of the cell, and the extracellular environment. A cell that has accumulated a large amount of DNA damage, or one that no longer effectively repairs damage incurred by its DNA, can enter one of three possible states: an irreversible state of dormancy, known as senescence; cell suicide, also known as apoptosis or programmed cell death or unregulated cell division, which can lead to the formation of a tumor.

### SUMMARY OF THE INVENTION

The present invention relates in part to the discovery that certain biological markers (referred to herein as “HNCMARKERS”), such as proteins, nucleic acids, polymorphisms, metabolites, and other analytes, as well as certain physiological conditions and states.

The invention provides a method of accessing the effectiveness of a treatment regimen treatment of a subject having a head and neck cancer by detecting the level of an effective amount of one or more HNCMARKERS in a sample from the subject, and comparing the level of the effective amount of the one or more HNCMARKERS to a reference value.

In another aspect the invention provides a method of monitoring a treatment regimen of a subject with head and neck cancer by detecting the level of an effective amount of one or more HNCMARKERS in a first sample from the subject at a first period of time and detecting the level of an effective amount of one or more HNCMARKERS in a second sample from the subject at a second period of time. The level of the effective amount of one or more HNCMARKERS detected in the first sample to the amount detected in the second sample, or reference value is compared.

In a further aspect, the invention provides method of determining whether a subject with head and neck cancer would derive a benefit from a treatment regimen by detecting the level of an effective amount of one or more HNCMARKERS comparing the level of the effective amount of one or more HNCMARKERS detected to a reference value.

In yet a further aspect, the invention provides a method for predicting the survivability of a head and neck cancer-diagnosed subject by detecting the level of an effective amount of one or more HNCMARKERS in a sample from the subject, and comparing the level of the effective amount of the one or more HNCMARKERS to a reference value.

In another aspect the invention provides method of determining the sensitivity of a head and neck cancer to a chemotherapeutic agent comprising identifying an alteration in at least one HNCMARKER. The presence of said alteration indicates said cell is sensitive to a chemotherapeutic agent.

In on aspect the invention provides a method of determining the resistance of a head and neck cancer to a chemotherapeutic agent comprising identifying an alteration in at least one HNCMARKER. The absence of said alteration indicates said cell is resistant to a chemotherapeutic agent.

The alteration is an increase or a decrease. The alteration is determined by detecting a mutation in a HNCMARKER or a post-translation modification of a HNCMARKER. Post-translational modifications include for example, phosphorylation, ubiquitination, sumo-ylation, acetylation, alkylation, methylation, glycylation, glycosylation, isoprenylation, lipoylation, phosphopantetheinylation, sulfation, selenation and C-terminal amidation.

The treatment regimen is immunotherapy such as Cetuximab, induction chemotherapy, concurrent chemoradiotherapy or a combination thereof. The chemotherapy or chemoradiotherapy comprises is carboplatin, or one of the related class of platinum drugs, taxane, or one of the class of taxanes, or both

The subject has received treated for head and neck cancer. For example, the subject has received immunotherapy, induction chemotherapy, concurrent chemoradiotherapy or a combination thereof. Alternatively, the subject has not received treatment for head and neck cancer.

Optionally, the methods of the invention further include measuring at least one standard parameters associated with a tumor. The level of a HNCMARKER is measured by immunohistochemistry.

The HNCMARKER is any marker disclosed herein. For example, the HNCMARKER is any marker listed on Table 1.

In some aspects the HNCMARKER is XPF, FANCD2, RAD51, BRCA1, ATM, PAR, p53, ERCC1, pH2AX, orpMK2.

In one aspect the HNCMARKER is:

a) XPF and at least one HNCMARKER selected from the group consisting of FANCD2, pMAPKAP Kinase 2 (pMK2), pH2AX, BRCA1, PAR, ATM, ERCC1, RAD51, p53, POL H, MUS81, p16, and HPV

b) FANCD2 and at least one HNCMARKER selected from the group consisting of XPF, pMAPKAP Kinase 2 (pMK2), pH2AX, BRCA1, PAR, ATM, ERCC1, RAD51, p53, POL H, MUS81, p16, and HPV.

c) pMAPKAP Kinase 2 (pMK2) and at least one HNCMARKER selected from the group consisting of XPF, FANCD2, pH2AX, BRCA1, PAR, ATM, ERCC1, RAD51, p53, POL H, MUS81, p16, and HPV.

d) pH2AX and at least one HNCMARKER selected from the group consisting of XPF, FANCD2, pMAPKAP Kinase 2 (pMK2), BRCA1, PAR, ATM, ERCC1, RAD51, p53, POL H, MUS81, p16, and HPV.

e) BRCA1 and at least one HNCMARKER selected from the group consisting of XPF, FANCD2, pMAPKAP Kinase 2 (pMK2), pH2AX, PAR, ATM, ERCC1, RAD51, p53, POL H, MUS81, p16, and HPV.

f) PAR and at least one HNCMARKER selected from the group consisting of XPF, FANCD2, pMAPKAP Kinase 2 (pMK2), pH2AX, BRCA1, ATM, ERCC1, RAD51, p53, POL H, MUS81, p16, and HPV.

g) ATM and at least one HNCMARKER selected from the group consisting of XPF, FANCD2, pMAPKAP Kinase 2 (pMK2), pH2AX, BRCA1, PAR, ERCC1, RAD51, p53, POL H, MUS81, p16, and HPV.

h) ERCC1 and at least one HNCMARKER selected from the group consisting of XPF, FANCD2, pMAPKAP Kinase 2 (pMK2), pH2AX, BRCA1, PAR, ATM, RAD51, p53, POL H, MUS81, p16, and HPV.

i) RAD51 and at least one HNCMARKER selected from the group consisting of XPF, FANCD2, pMAPKAP Kinase 2 (pMK2), pH2AX, BRCA1, PAR, ATM, ERCC1, p53, POL H, MUS81, p16, and HPV.

j) p53, and at least one HNCMARKER selected from the group consisting of XPF, FANCD2, pMAPKAP Kinase 2 (pMK2), pH2AX, BRCA1, PAR, ATM, ERCC1, RAD51, POL H, MUS81, p16, and HPV.

k) POL H and at least one HNCMARKER selected from the group consisting of XPF, FANCD2, pMAPKAP Kinase 2 (pMK2), pH2AX, BRCA1, PAR, ATM, ERCC1, RAD51, p53, MUS81, p16, and HPV.

l) MUS81 and at least one HNCMARKER selected from the group consisting of XPF, FANCD2, pMAPKAP Kinase 2 (pMK2), pH2AX, BRCA1, PAR, ATM, ERCC1, RAD51, p53, POL H, p16, and HPV.

m) p16 and at least one HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and HPV.

n) HPV and at least one HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and p16.

In another aspect the HNCMARKER is

a) XPF and at least two HNCMARKER selected from the group consisting of FANCD2, pMAPKAP Kinase 2 (pMK2), pH2AX, BRCA1, PAR, ATM, ERCC1, RAD51, p53, POL H, MUS81, p16, and HPV

b) FANCD2 and at least two HNCMARKER selected from the group consisting of XPF, pMAPKAP Kinase 2 (pMK2), pH2AX, BRCA1, PAR, ATM, ERCC1, RAD51, p53, POL H, MUS81, p16, and HPV.

c) pMAPKAP Kinase 2 (pMK2) and at least two HNCMARKER selected from the group consisting of XPF, FANCD2, pH2AX, BRCA1, PAR, ATM, ERCC1, RAD51, p53, POL H, MUS81, p16, and HPV.

d) pH2AX and at least two HNCMARKER selected from the group consisting of XPF, FANCD2, pMAPKAP Kinase 2 (pMK2), BRCA1, PAR, ATM, ERCC1, RAD51, p53, POL H, MUS81, p16, and HPV.

e) BRCA1 and at least two HNCMARKER selected from the group consisting of XPF, FANCD2, pMAPKAP Kinase 2 (pMK2), pH2AX, PAR, ATM, ERCC1, RAD51, p53, POL H, MUS81, p16, and HPV.

f) PAR and at least two HNCMARKER selected from the group consisting of XPF, FANCD2, pMAPKAP Kinase 2 (pMK2), pH2AX, BRCA1, ATM, ERCC1, RAD51, p53, POL H, MUS81, p16, and HPV.

g) ATM and at least two HNCMARKER selected from the group consisting of XPF, FANCD2, pMAPKAP Kinase 2 (pMK2), pH2AX, BRCA1, PAR, ERCC1, RAD51, p53, POL H, MUS81, p16, and HPV.

h) ERCC1 and at least two HNCMARKER selected from the group consisting of XPF, FANCD2, pMAPKAP Kinase 2 (pMK2), pH2AX, BRCA1, PAR, ATM, RAD51, p53, POL H, MUS81, p16, and HPV.

i) RAD51 and at least two HNCMARKER selected from the group consisting of XPF, FANCD2, pMAPKAP Kinase 2 (pMK2), pH2AX, BRCA1, PAR, ATM, ERCC1, p53, POL H, MUS81, p16, and HPV.

j) p53, and at least two HNCMARKER selected from the group consisting of XPF, FANCD2, pMAPKAP Kinase 2 (pMK2), pH2AX, BRCA1, PAR, ATM, ERCC1, RAD51, POL H, MUS81, p16, and HPV.

k) POL H and at least two HNCMARKER selected from the group consisting of XPF, FANCD2, pMAPKAP Kinase 2 (pMK2), pH2AX, BRCA1, PAR, ATM, ERCC1, RAD51, p53, MUS81, p16, and HPV.

l) MUS81 and at least two HNCMARKER selected from the group consisting of XPF, FANCD2, pMAPKAP Kinase 2 (pMK2), pH2AX, BRCA1, PAR, ATM, ERCC1, RAD51, p53, POL H, p16, and HPV.

m) p16 and at least two HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and HPV.

n) HPV and at least two HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and p16.

In a further aspect the HNCMARKER is

a) XPF and at least three HNCMARKER selected from the group consisting of FANCD2, pMAPKAP Kinase 2 (pMK2), pH2AX, BRCA1, PAR, ATM, ERCC1, RAD51, p53, POL H, MUS81, p16, and HPV

b) FANCD2 and at least three HNCMARKER selected from the group consisting of XPF, pMAPKAP Kinase 2 (pMK2), pH2AX, BRCA1, PAR, ATM, ERCC1, RAD51, p53, POL H, MUS81, p16, and HPV.

c) pMAPKAP Kinase 2 (pMK2) and at least three HNCMARKER selected from the group consisting of XPF, FANCD2, pH2AX, BRCA1, PAR, ATM, ERCC1, RAD51, p53, POL H, MUS81, p16, and HPV.

d) pH2AX and at least three HNCMARKER selected from the group consisting of XPF, FANCD2, pMAPKAP Kinase 2 (pMK2), BRCA1, PAR, ATM, ERCC1, RAD51, p53, POL H, MUS81, p16, and HPV.

e) BRCA1 and at least three HNCMARKER selected from the group consisting of XPF, FANCD2, pMAPKAP Kinase 2 (pMK2), pH2AX, PAR, ATM, ERCC1, RAD51, p53, POL H, MUS81, p16, and HPV.

f) PAR and at least three HNCMARKER selected from the group consisting of XPF, FANCD2, pMAPKAP Kinase 2 (pMK2), pH2AX, BRCA1, ATM, ERCC1, RAD51, p53, POL H, MUS81, p16, and HPV.

g) ATM and at least three HNCMARKER selected from the group consisting of XPF, FANCD2, pMAPKAP Kinase 2 (pMK2), pH2AX, BRCA1, PAR, ERCC1, RAD51, p53, POL H, MUS81, p16, and HPV.

h) ERCC1 and at least three HNCMARKER selected from the group consisting of XPF, FANCD2, pMAPKAP Kinase 2 (pMK2), pH2AX, BRCA1, PAR, ATM, RAD51, p53, POL H, MUS81, p16, and HPV.

i) RAD51 and at least three HNCMARKER selected from the group consisting of XPF, FANCD2, pMAPKAP Kinase 2 (pMK2), pH2AX, BRCA1, PAR, ATM, ERCC1, p53, POL H, MUS81, p16, and HPV.

j) p53, and at least three HNCMARKER selected from the group consisting of XPF, FANCD2, pMAPKAP Kinase 2 (pMK2), pH2AX, BRCA1, PAR, ATM, ERCC1, RAD51, POL H, MUS81, p16, and HPV.

k) POL H and at least three HNCMARKER selected from the group consisting of XPF, FANCD2, pMAPKAP Kinase 2 (pMK2), pH2AX, BRCA1, PAR, ATM, ERCC1, RAD51, p53, MUS81, p16, and HPV.

l) MUS81 and at least three HNCMARKER selected from the group consisting of XPF, FANCD2, pMAPKAP Kinase 2 (pMK2), pH2AX, BRCA1, PAR, ATM, ERCC1, RAD51, p53, POL H, p16, and HPV.

m) p16 and at least three HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and HPV.

n) HPV and at least three HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and p16.

In another aspect the HNCMARKER is any marker(s) enumerated on Tables 2-10.

Also provided by the invention is an algorithm that is derived from the list of biomarkers in Table 1 and Table 2 which specifies how the biomarkers are associated in relation to the other biomarkers in the panel, such that the biomarker algorithm indicates a predictive or prognostic value in treatment response of head and neck cancer

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 Association of scoring by pathologist Intensity and Quantity values versus machine assisted image analysis and quantitation. Comparisons are made between alternative scoring strategies for immunohistochemistry with the XPF DNA repair biomarker for each head and neck cancer patient. Machine scoring includes Percentage Nuclei (% N), Percentage Nuclei X Average Intensity (% N×Av I), and % Nuclei×Intensity (% N×I). Pathologist scores were by Intensity (I) or Quantity (Q). Correlation plots as shown are computed for the similarity by an R² value. R² values range from 0.744−0.839 in these correlations.

FIG. 2 Marker output variations between patients far exceed the intersample variability in head and neck cancer. A, Theoretical definition of the calculation for core-core variability and rank change assessment; B, Table indicating the average error and N number of patients being evaluated for HNCMARKERS; C, Results from patient ranking for four HNCMARKERS. Patient marker scores are sorted from lowest to highest, and core-core variance per patient is displayed as a vertical dashed line.

FIG. 3. Partition analysis for all 1-, 2-, 3-, and 4-marker models of HNCMARKERS in tests of discrimination of Head and Neck cancer patient Overall Survival following concurrent chemoradiotherapy. A partition analysis was calculated for the HNCMARKERS in the study in examples of 1-, 2-, 3-, and 4-marker models. Shown are the distributions of all models of these types as determined for the following statistical parameters: p value, Positive predictive value, Relative risk, and Average Error Rate (AER). The median value for all the models of each type is illustrated by a convergence to the box plots, and the range of values is shown by the brackets. Dark shaded boxes indicate the 95% values for each distribution of models. It is evident that for these four statistical parameters illustrate an improvement by increasing the marker number in the algorithm such that 1<2<3<4 in statistical power.

FIG. 4. Root marker performance improved in multimarker models for overall survival following treatment with concurrent chemoradiotherapy. A partition analysis was calculated for the HNCMARKERS in the study with targeted start points of specific single biomarkers. In the example shown, there are five root markers, FANCD2, XPF, BRCA1, ATM and RAD51, that were calculated. Shown are the starting 1-marker models and then all 2-, 3-, and 4-marker models that always will contain the same root marker. In each case, the computed log 10 P-value (squares), Positive Predictive Value (PPV) (triangles) and AER (black circles) are shown for each Root Marker alone, and in combination with other HNCMARKERS in 2-, 3- and 4-marker models. The median values of all the models are plotted for each model.

FIG. 5. Partition analysis for HNCMARKERS multimarker algorithms in predicting overall survival in Head and Neck cancer patients treated with concurrent chemoradiotherapy. Two examples of the role of a root marker in specific marker combinations are illustrated by comparison of selected 2-, 3- and 4-marker models. Statistical analysis shown is taken from data tables, highlighting the Kaplan-Meier survival curves for the HNCMARKERS in the group. P-values are inserted in each plot and black dashed line is the trend for all patients in the study. Example 1, The Root marker is PAR, and the markers added in 2-, 3-, and 4-marker combinations are RAD51, XPF, and FANCD2. Example 2, The Root marker is pMK2, and the markers added in 2-, 3-, and 4-marker combinations are BRCA1, FANCD2, and p53.

FIG. 6. Probability Analysis Schematic. Probability analysis is a computational process that allows for a continuous scoring of the HNCMARKER outputs. In the algorithm, a region of low incidence of death and a region of high incidence of death is proposed from estimates of the probability density distributions. For the High survival (ie. Disease free-survival or overall survival) and Low survival groups, a single score reflecting group membership is constructed from the individual group probabilities. Similar analysis is operational for additional endpoints, such as recurrence.

FIG. 7. Single HNCMARKER Probability Analysis on Head and Neck cancer patients treated with chemoradiotherapy. An example HNCMARKER, XPF, is shown indicating the projections for the Scores by Outcomes, Kaplan-Meier disease-specific survival Curve, Predicted Outcome from Score, ROC Plot from Score from the Probability Analysis and statistical calculations. For the Kaplan-Meier projection of the outcomes for survival, HIGH, High Survival Subgroup, LOW, Low DSS subgroup. Black dashed line, ALL Patients

FIG. 8. Probability prediction of Single HNCMARKERS for overall survival for patients treated with concurrent chemoradiotherapy The example shows five biomarkers in the study in univariate probability analysis (XPF, FANCD2, BRCA1, ATM, and RAD51). Kaplan-Meier Survival Curves, HIGH and LOW refer to the patient subgrouping into High overall survival rate (Good Outcome) and Low overall survival rate (Poor Outcome) respectively. Additional data with other statistical parameters are in Table 6.

FIG. 9. Probability Analysis of the DNA Repair HNCMARKERS on all 1-, 2-, 3-, and 4-HNCMARKER models for overall survival of Head and Neck cancer patients following treatment with concurrent chemoradiotherapy. The markers in the analysis included the group of HNCMARKERS. All 1-marker, 2-marker, 3-marker, and 4-, marker combinations were compared and plotted on x-axis as 1, 2, 3, or 4. The median value of all models in the group is represented by a narrow white box is the center region of each plotted value. Black box denotes 95% confidence interval for the median. Outside white box denotes the middle half of the data (white part above median is quarter of data, white part below median is quarter of data. The statistical values assessed were Fraction Sample Assigned, AUC, Sensitivity, and Specificity.

FIG. 10. Probability analysis of Root Marker Performance. A probability analysis was calculated for the HNCMARKERS in the study with targeted start points of specific single biomarkers. In the example shown, there are five root markers, FANCD2, XPF, BRCA1, ATM and RAD51, that were calculated. Shown are the starting 1-marker models and then all 2-, 3-, and 4-marker models that always will contain the same root marker. In each case, the computed log 10 P-value (squares), Positive Predictive Value (PPV) (triangles) and AER (black circles) are shown for each Root Marker alone, and in combination with other HNCMARKERS in 2-, 3- and 4-marker models. The median values of all the models are plotted for each model

FIG. 11. Probability analysis demonstration of reduced confusion by multimarker models. The example shown illustrates the role of HNCMARKERS in diminishing the fraction of the total patients in a study group where the results of the test are confused, meaning that there is ambiguity as to whether the patient is in a good survival or poor survival group. The Outcome Score is developed from Probability analysis with the range from +1 to −1. Each patient evaluated in listed as an entry on the X-axis. Dark Vertical lines indicate 95% Confidence Intervals for all 1-HNCMARKER models (left) and 4-HNCMARKER models, and dashed lines indicate the patient ranges for the tests. The boxed areas denote the fraction of patients with a confused result of the test within the 95% confidence intervals. The four HNCMARKER models significantly reduced the confused group in the patient cohort.

FIG. 12. Probability Analysis of a Three Marker Model—RAD51, BRCA1, FANCD2—for overall survival for Head and Neck cancer patients being treated with concurrent chemoradiotherapy. Several different statistical outputs are illustrated to demonstrate the effect of HNCMARKER combinations for assessing clinical data. Scores by Outcome, patients are separated by those with an event (death) or no event (survival) and the probability of correctly calling the result of the test with the four marker test is plotted from a scale of −1.0 to +1.0. Kaplan-Meier Survival Curves, HIGH and LOW refer to the patient subgrouping into High overall survival rate (Good Outcome) and Low overall survival rate (Poor Outcome) respectively. Black dashed line indicates the trend for all of the patients in the evaluation. Predicted Outcome from Score, is shown by plotting the likelihood of an event (death) against the probability score (95% confidence intervals with dashed lines); ROC Plot from Score, Area Under Curve (AUC) sensitivity/specificity determination listed, values range from 0-1.

FIG. 13. Probability Analysis of a Four Marker Model for overall survival for Head and Neck cancer patients being treated with from concurrent chemoradiotherapy. The four HNCMARKERS used in this example are XPF, FANCD2, BRCA1, and ATM. Several different statistical outputs are illustrated to demonstrate the effect of HNCMARKER combinations for assessing clinical data. Scores by Outcome, patients are separated by those with an event (death) or no event (survival) and the probability of correctly calling the result of the test with the four marker test is plotted from a scale of −1.0 to +1.0. Kaplan-Meier Survival Curves, HIGH and LOW refer to the patient subgrouping into High overall survival rate (Good Outcome) and Low overall survival rate (Poor Outcome) respectively. Black dashed line indicates the trend for all of the patients in the evaluation. Predicted Outcome from Score, is shown by plotting the likelihood of an event (death) against the probability score (95% confidence intervals with dashed lines); ROC Plot from Score, Area Under Curve (AUC) sensitivity/specificity determination listed, values range from 0-1.

FIG. 14. Probability Analysis of a Four Marker Model for overall survival for Head and Neck cancer patients being treated with concurrent chemoradiotherapy. The four HNCMARKERS used in this example are XPF, FANCD2, RAD51, and ATM. Several different statistical outputs are illustrated to demonstrate the effect of HNCMARKER combinations for assessing clinical data. Scores by Outcome, patients are separated by those with an event (death) or no event (survival) and the probability of correctly calling the result of the test with the four marker test is plotted from a scale of −1.0 to +1.0. Kaplan-Meier Survival Curves, HIGH and LOW refer to the patient subgrouping into High overall survival rate (Good Outcome) and Low overall survival rate (Poor Outcome) respectively. Black dashed line indicates the trend for all of the patients in the evaluation. Predicted Outcome from Score, is shown by plotting the likelihood of an event (death) against the probability score (95% confidence intervals with dashed lines); ROC Plot from Score, Area Under Curve (AUC) sensitivity/specificity determination listed, values range from 0-1.

FIG. 15. HNCMARKER models are significantly discriminating patient Disease-specific survival subgroups in Head and Neck cancer treatment. An alternative clinical parameter was assessed. Disease-Specific Survival (DSS) The example shows the Partition analysis calculation for single HNCMARKERS with statistical outputs of log 10 p-values. For four of the markers exemplified, the values were XPF (p=1.74e-5), BRCA1 (p=8.5e-4), FANCD2 (p=4.58e-4), and RAD51 (p=0.0013) HIGH, High Survival Subgroup, LOW, Low DSS subgroup. Black dashed line, ALL Patients

FIG. 16. Four HNCMARKER models for distinguishing benefit from chemoradiotherapy by monitoring the Disease-Specific Survival of Head and Neck cancer patients. An alternative clinical parameter was assessed, Disease-Specific Survival (DSS) with an example of one Four HNCMARKER model composed of BRCA1, XPF, RAD51, and FANCD2. Kaplan-Meier Disease-specific survival curves were plotted and the significance between HIGH survival and LOW survival groups assessed by log 10 p-value. Partition analysis on left, log 10 p-value=2.9e-6. Probability analysis on right, log 10 p-value=5.35e-4. HIGH, High Survival Subgroup, LOW, Low DSS subgroup. Black dashed line, ALL Patients

FIG. 17. XPF univariate analysis shows improved response prediction to induction chemotherapy in head and neck cancer. The chart shows that univariate analysis of the XPF biomarker scores relative to the discrimination between Responder subgroups (CR and PR) and Stable Disease (SD). Low XPF score showed a 100% response rate to induction chemotherapy treatment. Of the 37 patients in the study, 11 had complete response, 19 had partial response, and 7 had stable disease.

FIG. 18. Two HNCMARKER analysis for prediction of success from induction chemotherapy in head and neck cancer. Two examples of DNA repair biomarker comparisons are shown in pairwise combinations with these three markers: XPF, pMK2, pH2AX. Triangles, SD patients; Circles, CR/PR patients. Patients are separated by partition analysis. Dotted-square indicates SD-containing group in which there is a 100% SD group prediction for both markers. Lower quadrant indicates an AUC value for the pairwise combination.

FIG. 19. Two marker analysis of HNCMARKERS in Induction Chemotherapy prediction in Head and Neck Cancer: alternative sphere discriminant analysis. NE05 (XPF), DR07 (pH2AX), DR02 (pMK2) HNCMARKERS are evaluated in two marker algorithms with each other, where the centers of the two concentric ellipses are compared for CR/PR (responders) and SD (non-responders). Patients group into the closest association with one group or the other have the greatest likelihood of being correctly assigned by the two-marker algorithm.

FIG. 20. Multiple HNCMARKER benefit for patient response to induction chemotherapy in head and neck cancer. The intent of the DNA repair biomarker panels was to discriminate between Responder subgroups (CR and PR) and Stable Disease (SD). In order to show the value of the biomarker and/or 2- or 3-biomarker panels in identifying patients that are truly responders, the calculations were made where there were no errors in calling the Stable Disease patients correctly (100% Stable Disease/Progressive Disease correct). In this manner, all 1-marker models were compared with all 2-marker and 3-marker models. 2-marker models were M1+M2 (additive) and M1/M2 (ratio). 3-marker models were M1+M2+M3 (additive) and M1/M2+M3 (ratio+additive). Results are computed as combined AUC and ranked based on the fraction of CR/PR (responders) called correctly.

FIG. 21. Classification tree analysis of head and neck cancer response to induction chemotherapy regarding HNCMARKERS. Decision points for each node of the Classification tree are made by recursive partitioning where each split maximally distinguishes the response subgroups (SD vs CR/PR). Splitting is continued until the stopping group size is reached (group size=1 will yield perfect classification). Pruning removes the least explanatory branches based on cost-complexity objective function. For this decision tree, a 4-marker model gives the best 5 node separation of the patient response categories. The DNA repair and response biomarkers are XPF, pMK2, PAR, and pH2AX representing several of the DNA repair pathways. The figure shows the distribution of patients at each node. Part b, Confusion matrix for 5 node HNCMARKER model pruned classification tree from induction chemotherapy. The method compares the Predicted distribution of patient response with the Actual distribution. There is a non random selection of 5 nodes based on the decrease in error for each successive size tree. Note that the 5 node model shows about ½ the total variance described and is a good ‘rule of thumb’ for simplification.

### DETAILED DESCRIPTION OF THE INVENTION

The present invention relates to the identification of biomarkers associated with head and neck cancer. Specifically, these biomarkers are proteins associated in DNA repair pathways. DNA repair pathways are important to the cellular response network to chemotherapy and radiation. Tumor cells have altered DNA repair and DNA damage response pathways and that loss of one of these pathways renders the cancer more sensitive to a particular class of DNA damaging agents. Cancer therapy procedures such as chemotherapy and radiotherapy work by overwhelming the capacity of the cell to repair DNA damage, resulting in cell death.

There are six major DNA repair pathways distinguishable by several criteria which can be divided into three groups those that repair single strand damage and those that repair double stand damage. Single stranded damage repair pathways include Base-Excision Repair (BER); Nucleotide Excision Repair (NER); Mismatch Repair (MMR); Homologous Recombination/Fanconi Anemia pathway (HR/FA); Non-Homologous Endjoining (NHEJ), and Translesion DNA Synthesis repair (TLS).

BER, NER and MMR repair single strand DNA damage. When only one of the two strands of a double helix has a defect, the other strand can be used as a template to guide the correction of the damaged strand. In order to repair damage to one of the two paired molecules of DNA, there exist a number of excision repair mechanisms that remove the damaged nucleotide and replace it with an undamaged nucleotide complementary to that found in the undamaged DNA strand. BER repairs damage due to a single nucleotide caused by oxidation, alkylation, hydrolysis, or deamination. NER repairs damage affecting longer strands of 2-30 bases. This process recognizes bulky, helix-distorting changes such as thymine dimers as well as single-strand breaks (repaired with enzymes such UvrABC endonuclease). A specialized form of NER known as Transcription-Coupled Repair (TCR) deploys high-priority NER repair enzymes to genes that are being actively transcribed. MMR corrects errors of DNA replication and recombination that result in mispaired nucleotides following DNA replication.

NHEJ and HR repair double stranded DNA damage. Double stranded damage is particularly hazardous to dividing cells. The NHEJ pathway operates when the cell has not yet replicated the region of DNA on which the lesion has occurred. The process directly joins the two ends of the broken DNA strands without a template, losing sequence information in the process. Thus, this repair mechanism is necessarily mutagenic. However, if the cell is not dividing and has not replicated its DNA, the NHEJ pathway is the cell's only option. NHEJ relies on chance pairings, or microhomologies, between the single-stranded tails of the two DNA fragments to be joined. There are multiple independent “failsafe” pathways for NHEJ in higher eukaryotes. Recombinational repair requires the presence of an identical or nearly identical sequence to be used as a template for repair of the break. The enzymatic machinery responsible for this repair process is nearly identical to the machinery responsible for chromosomal crossover during meiosis. This pathway allows a damaged chromosome to be repaired using the newly created sister chromatid as a template, i.e. an identical copy that is also linked to the damaged region via the centromere. Double-stranded breaks repaired by this mechanism are usually caused by the replication machinery attempting to synthesize across a single-strand break or unrepaired lesion, both of which result in collapse of the replication fork.

Translesion synthesis is an error-prone (almost error-guaranteeing) last-resort method of repairing a DNA lesion that has not been repaired by any other mechanism. The DNA replication machinery cannot continue replicating past a site of DNA damage, so the advancing replication fork will stall on encountering a damaged base. The translesion synthesis pathway is mediated by specific DNA polymerases that insert extra bases at the site of damage and thus allow replication to bypass the damaged base to continue with chromosome duplication. The bases inserted by the translesion synthesis machinery are template-independent, but not arbitrary; for example, one human polymerase inserts adenine bases when synthesizing past a thymine dimer.

Both normal cellular processes and exogenous agents contribute to the accumulation of DNA damage for which eukaryotic cells have evolved complex and redundant repair mechanisms to ensure stability and high fidelity replication of the genetic material. While spontaneous mutations cannot entirely account for the lifetime cancer risk, defects in DNA repair can lead to a ‘mutator’ phenotype where cells accumulate damage at an accelerated rate, leading to oncogenesis. While these defects may contribute to genomic instability and aggressiveness, they might also sensitize tumor cells to damage by exogenous DNA damaging agents such as chemotherapy and ionizing radiation. Thus, because DNA damage repair defects are more likely to be prevalent in cancer cells and relate to aggressiveness, the cellular DNA repair machinery offers an opportunity for prediction and prognosis as well as a set of targets for therapeutic development.

Accordingly, the invention provides methods of determining the responsiveness, e.g., sensitivity or resistance, of a cancer cell to a therapeutic agent (e.g. chemotherapy) or ionizing radiation by determining which DNA repair pathway is altered. These methods are also useful for monitoring subjects undergoing treatments and therapies for cancer or other cell proliferative disorders, and for selecting therapies and treatments that would be efficacious in subjects having cancer or other cell proliferative disorders, wherein selection and use of such treatments and therapies slow the progression of cancer or other cell proliferative disorders. More specifically, the invention provides methods of determining the whether a patient with a head and neck cancer will be responsive to induction chemotherapy.

### DEFINITIONS

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as “clinical parameters” defined herein, as well as “traditional laboratory risk factors”, also defined herein. Biomarkers also include any calculated indices created mathematically or combinations of any one or more of the foregoing measurements, including temporal trends and differences. Where available, and unless otherwise described herein, biomarkers which are gene products are identified based on the official letter abbreviation or gene symbol assigned by the international Human Genome Organization Naming Committee (HGNC) and listed at the date of this filing at the US National Center for Biotechnology Information (NCBI) web site.

“HNCMARKER” OR “HNCMARKERS” encompass one or more of all nucleic acids or polypeptides whose levels are changed in a subject in response to a therapy. As used herein HNCMARKERS includes XPF, FANCD2, pMAPKAP Kinase 2 (pMK2), pH2AX, BRCA1, PAR, PARP1, MLH1, ATM, ERCC1, RAD51, pHSP27, p53, POL H, MUS81, Ki67, p16, and HPV. Individual HNCMARKERS are collectively referred to herein as, inter alia, “head and neck cancer-associated proteins”, “HNCMARKER polypeptides”, or “HNCMARKER proteins”. The corresponding nucleic acids encoding the polypeptides are referred to as “head and neck cancer-associated nucleic acids”, “head and neck cancer-associated genes”, “HNCMARKER nucleic acids”, or “HNCMARKER genes”. Unless indicated otherwise, “HNCMARKER”, “head and neck cancer-associated proteins”, “head and neck cancer-associated nucleic acids” are meant to refer to any of the biomarkers disclosed herein, e.g XPF, FANCD2, pMAPKAP Kinase 2 (pMK2), pH2AX, BRCA1, PAR, PARP1, MLH1, ATM, ERCC1, RAD51, pHSP27, p53, POL H, MUS81, Ki67, p16, and HPV.

The corresponding metabolites of the HNCMARKER proteins or nucleic acids can also be measured, as well as any of the aforementioned traditional risk marker metabolites.

Physiological markers of health status (e.g., such as age, family history, and other measurements commonly used as traditional risk factors) are referred to as “HNCMARKER physiology”. Calculated indices created from mathematically combining measurements of one or more, preferably two or more of the aforementioned classes of HNCMARKERS are referred to as “HNCMARKER indices”.

A “Clinical indicator” is any physiological datum used alone or in conjunction with other data in evaluating the physiological condition of a collection of cells or of an organism. This term includes pre-clinical indicators.

“Clinical parameters” encompasses all non-sample or non-analyte biomarkers of subject health status or other characteristics, such as, without limitation, age (Age), ethnicity (RACE), gender (Sex), or family history (FamHX).

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining HNCMARKERS and other biomarkers are linear and non-linear equations and statistical classification analyses to determine the relationship between levels of HNCMARKERS detected in a subject sample and the subject's responsiveness to chemotherapy. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a HNCMARKER selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935. Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, as in the responsiveness to treatment, cancer recurrence or survival and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation” or “evaluation of risk” in the context of the present invention encompasses making a prediction of the probability, odds, or likelihood that an event or disease state may occur, the rate of occurrence of the event or conversion from one disease state. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the responsiveness to treatment thus diagnosing and defining the risk spectrum of a category of subjects defined as being responders or non-responders. In the categorical scenario, the invention can be used to discriminate between normal and other subject cohorts at higher risk for responding. Such differing use may require different HNCMARKER combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopsies, whole blood, serum, plasma, blood cells, endothelial cells, lymphatic fluid, ascites fluid, interstitial fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, or any other secretion, excretion, or other bodily fluids. A “sample” may include a single cell or multiple cells or fragments of cells. The sample is also a tissue sample. The sample is or contains a circulating endothelial cell or a circulating tumor cell. The sample includes a primary tumor cell, primary tumor, a recurrent tumor cell, or a metastatic tumor cell.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is considered highly significant at a p-value of 0.05 or less. Preferably, the p-value is 0.04, 0.03, 0.02, 0.01, 0.005, 0.001 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of cancer. A subject can be male or female.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms. Traditional laboratory risk factors for tumor recurrence include for example Proliferative index, tumor infiltrating lymphocytes. Other traditional laboratory risk factors for tumor recurrence known to those skilled in the art.

### Methods and Uses of the Invention

The methods disclosed herein are used with subjects undergoing treatment and/or therapies for a head and neck cancer, subjects who are at risk for developing a reoccurrence of head and neck cancer, and subjects who have been diagnosed with head and neck cancer and The methods of the present invention are to be used to monitor or select a treatment regimen for a subject who has a head and neck cancer, and to evaluate the predicted survivability and/or survival time of a head and neck cancer-diagnosed subject. Treatment regimens include for example but not limited to induction therapy or concurrent therapy, and combinations of thereof.

Responsiveness (e.g., resistance or sensitivity) of a cell to DNA damage agents such as a chemotherapeutic agent or ionizing radiation is determined by measuring an effective amount of a HNCMARKER proteins, nucleic acids, polymorphisms, metabolites, and other analytes (which may be two or more) in a test sample (e.g., a subject derived sample), and comparing the effective amounts to reference or index values, often utilizing mathematical algorithms or formula in order to combine information from results of multiple individual HNCMARKERS and from non-analyte clinical parameters into a single measurement or index. The cell is for example a cancer cell. Optionally, the cancer is a head or neck cancer such as cancer of the nasal cavity, sinuses, lips, mouth, salivary glands, throat, or larynx [voice box] The HNCMARKERs is for example, XPF, FANCD2, pMK2, PAR, MLH1, PARP1, pH2AX, pHSP27, BRCA1, RAD51, ERCC1, p53, p16, HPV

By resistance is meant that the failure of a cell to respond to an agent. For example, resistance to a chemotherapeutic drug or ionizing radiation means the cell is not damaged or killed by the drug. By sensitivity is meant that that the cell responds to an agent. For example, sensitivity to a chemotherapeutic drug or radiation means the cell is damaged or killed by the drug. For example, responsiveness of a cell to a chemotherapeutic agent or ionizing radiation identified by identifying a decrease in expression or activity one or more HNCMARKERS. The presence of a deficiency in HNCMARKER indicates that the cell is sensitive to a chemotherapeutic agent or ionizing radiation. Whereas, the absence of a deficiency indicates that the cell is resistant to a chemotherapeutic agent or ionizing radiation.

The methods of the present invention are useful to treat, alleviate the symptoms of, monitor the progression of or delay the onset of head and neck cancer in a subject.

Preferably, the methods of the present invention are used to identify and/or diagnose subjects who are asymptomatic for a head and neck cancer recurrence. “Asymptomatic” means not exhibiting the traditional symptoms.

The methods of the present invention are also useful to identify and/or diagnose subjects already at higher risk of developing a head and neck cancer or based on solely on the traditional risk factors.

Expression of an effective amount of HNCMARKER proteins, nucleic acids or metabolites also allows for determination of whether a subject will derive a benefit from a particular course of treatment. In this method, a biological sample is provided from a subject before undergoing treatment, e.g., chemotherapeutic or concurrent chemoradiotherapy treatment, for head and neck cancer. By “derive a benefit” it is meant that the subject will respond to the course of treatment. By responding is meant that the treatment that there is a decrease in size, prevalence, or metastatic potential of a head and neck cancer in a subject. When treatment is applied prophylactically, “responding” means that the treatment retards or prevents a head and neck cancer or a head and neck cancer recurrence from forming or retards, prevents, or alleviates a symptom of clinical head and neck cancer. Assessment of head and neck cancers are made using standard clinical protocols.

Expression of an effective amount of HNCMARKER proteins, nucleic acids or metabolites also allows for the course of treatment of head and neck cancer to be monitored. In this method, a biological sample is provided from a subject undergoing treatment, e.g., chemotherapeutic or concurrent chemoradiotherapy treatment, for head and neck cancer.

If desired, biological samples are obtained from the subject at various time points before, during, or after treatment. Expression of an effective amount of HNCMARKER proteins, nucleic acids or metabolites is then determined and compared to a reference value are then identified, e.g. a control individual or population whose head and neck cancer state is known or an index value. The reference sample or index value may be taken or derived from one or more individuals who have been exposed to the treatment. Alternatively, the reference sample or index value may be taken or derived from one or more individuals who have not been exposed to the treatment. For example, samples may be collected from subjects who have received initial treatment for head and neck cancer disorder and subsequent treatment for diabetes to monitor the progress of the treatment.

A reference value can be relative to a number or value derived from population studies, including without limitation, such subjects having the same cancer, subject having the same or similar age range, subjects in the same or similar ethnic group, subjects having family histories of cancer, or relative to the starting sample of a subject undergoing treatment for a cancer. Such reference values can be derived from statistical analyses and/or risk prediction data of populations obtained from mathematical algorithms and computed indices of cancer recurrence. Reference HNCMARKER indices can also be constructed and used using algorithms and other methods of statistical and structural classification.

In one embodiment of the present invention, the reference value is the amount of HNCMARKERS in a control sample derived from one or more subjects who are responsive to chemotherapy in head and neck cancer. In another embodiment of the present invention, the reference value is the amount of HNCMARKERS in a control sample derived from one or more subjects who have higher disease free or overall survival rate from head and neck cancer. In the other embodiment of the present invention, the reference value is the amount of HNCMARKERS in a control sample derived from one or more subjects who are not at risk or at low risk for developing a recurrence of a head and neck cancer. In a further embodiment, such subjects are monitored and/or periodically retested for a diagnostically relevant period of time (“longitudinal studies”) following such test to verify continued absence of a head and neck cancer (disease free or overall survival). Such period of time may be one year, two years, two to five years, five years, five to ten years, ten years, or ten or more years from the initial testing date for determination of the reference value. Furthermore, retrospective measurement of HNCMARKERS in properly banked historical subject samples may be used in establishing these reference values, thus shortening the study time required.

A reference value can also comprise the amounts of HNCMARKERS derived from subjects who show an improvement in risk factors as a result of treatments and/or therapies for the cancer. A reference value can also comprise the amounts of HNCMARKERS derived from subjects who show an improvement in responsiveness to therapy as a result of treatments and/or therapies for the cancer. A reference value can also comprise the amounts of HNCMARKERS derived from subjects who have higher disease free/overall rate, or are at high risk for developing head and neck cancer, or who have suffered from head and neck cancer.

In another embodiment, the reference value is an index value or a baseline value. An index value or baseline value is a composite sample of an effective amount of HNCMARKERS from one or more subjects who do not have a head and neck cancer or subjects who are asymptomatic a head and neck cancer. A baseline value can also comprise the amounts of HNCMARKERS in a sample derived from a subject who has shown an improvement in head and neck cancer responsiveness to therapy or disease free/overall survival rate as a result of cancer treatments or therapies. In this embodiment, to make comparisons to the subject-derived sample, the amounts of HNCMARKERS are similarly calculated and compared to the index value. Optionally, subjects identified as having head and neck cancer, or being at increased risk of developing a head and neck cancer are chosen to receive a therapeutic regimen to slow the progression the cancer, or decrease or prevent the risk of developing a head and neck cancer.

The progression of a head and neck cancer, or effectiveness of a cancer treatment regimen can be monitored by detecting a HNCMARKER in an effective amount (which may be two or more) of samples obtained from a subject over time and comparing the amount of HNCMARKERS detected. For example, a first sample can be obtained prior to the subject receiving treatment and one or more subsequent samples are taken after or during treatment of the subject. The cancer is considered to be progressive (or, alternatively, the treatment does not prevent progression) if the amount of HNCMARKER changes over time relative to the reference value, whereas the cancer is not progressive if the amount of HNCMARKERS remains constant over time (relative to the reference population, or “constant” as used herein). The term “constant” as used in the context of the present invention is construed to include changes over time with respect to the reference value.

Additionally, therapeutic or prophylactic agents suitable for administration to a particular subject can be identified by detecting one or more of the HNCMARKERS in an effective amount (which may be two or more) in a sample obtained from a subject, exposing the subject-derived sample to a test compound that determines the amount (which may be two or more) of HNCMARKERS in the subject-derived sample. Accordingly, treatments or therapeutic regimens for use in subjects having a cancer, or subjects with non-responsiveness to therapy or lower disease free/overall survival rate can be selected based on the amounts of HNCMARKERS in samples obtained from the subjects and compared to a reference value. Two or more treatments or therapeutic regimens can be evaluated in parallel to determine which treatment or therapeutic regimen would be the most efficacious for use in a subject to delay onset, or slow progression of the cancer.

The present invention further provides a method for screening for changes in marker expression associated with head and neck cancer, by determining one or more of the HNCMARKERS in a subject-derived sample, comparing the amounts of the HNCMARKERS in a reference sample, and identifying alterations in amounts in the subject sample compared to the reference sample.

If the reference sample, e.g., a control sample, is from a subject that does not have a head and neck cancer, from cells that are sensitive to a therapeutic compound or radiation, or if the reference sample reflects a value that is relative to a person that has a high likelihood of responsiveness to the therapy, low risk of developing recurrence or higher rate of disease free/overall survival, a similarity in the amount of the HNCMARKER in the test sample and the reference sample indicates that the treatment is efficacious. However, a difference in the amount of the HNCMARKER in the test sample and the reference sample indicates a less favorable clinical outcome or prognosis. In contrast, if the reference sample, e.g., a control sample is from cells that are resistant to a therapeutic compound or radiation, or if the reference sample reflects a value that is relative to a person that has a high likelihood of non-responsiveness to the therapy, high risk of developing a recurrence or lower rate of disease free/overall survival, then a similarity in the amount of the HNCMARKER proteins in the test sample and the reference sample indicates that the treatment with that compound will result in a less favorable clinical outcome or prognosis. However, a change in the amount of the HNCMARKER in the test sample and the reference sample indicates that treatment with that therapeutic compound will be efficacious.

By “efficacious”, it is meant that the treatment leads to a decrease in the amount or activity of a HNCMARKER protein, nucleic acid, polymorphism, metabolite, or other analyte. Assessment of the risk factors disclosed herein can be achieved using standard clinical protocols. Efficacy can be determined in association with any known method for diagnosing, identifying, or treating a head and neck cancer.

The present invention also comprises a kit with a detection reagent that binds to two or more of the HNCMARKERS proteins, nucleic acids, polymorphisms, metabolites, or other analytes. Also provided by the invention is an array of detection reagents, e.g., antibodies and/or oligonucleotides that can bind to two or more HNCMARKER proteins or nucleic acids, respectively.

Also provided by the present invention is a method for treating one or more subjects with non-responsiveness to therapy or lower rate of lower rate of disease free/overall survival in a head and neck cancer by detecting the presence of altered amounts of an effective amount of the HNCMARKERS present in a sample from the one or more subjects; and treating the one or more subjects with one or more cancer-modulating drugs until altered amounts or activity of the HNCMARKERS return to a baseline value measured in one or more subjects with improvement in response to therapy or higher rate of disease free/overall survival.

Also provided by the present invention is a method for evaluating changes in the responsiveness to therapy or the rate of disease free/overall survival in a subject diagnosed with cancer, by detecting an effective amount of the HNCMARKERS (which may be two or more) in a first sample from the subject at a first period of time, detecting the amounts of the HNCMARKERS in a second sample from the subject at a second period of time, and comparing the amounts of the HNCMARKERS detected at the first and second periods of time.

### Diagnostic, Predictive, and Prognostic Indications of the Invention

The amount of the HNCMARKER protein, nucleic acid, polymorphism, metabolite, or other analyte can be measured in a test sample and compared to the “normal control level,” utilizing techniques such as reference limits, discrimination limits, or risk defining thresholds to define cutoff points and abnormal values. Such normal control level and cutoff points may vary based on whether a HNCMARKER is used alone or in a formula combining with other HNCMARKERS into an index. Alternatively, the normal control level can be a database of HNCMARKER patterns from previously tested subjects who responded to chemotherapy (e.g., induction chemotherapy, concurrent chemoradiotherapy, or both radiation therapy over a clinically relevant time horizon.

The present invention may be used to make continuous or categorical measurements of the response to chemotherapy or cancer survival, thus diagnosing and defining the risk spectrum of a category of subjects defined as at risk for not responding to chemotherapy. In the categorical scenario, the methods of the present invention can be used to discriminate between treatment responsive and treatment non-responsive subject cohorts. In other embodiments, the present invention may be used so as to discriminate those who have an improved survival potential. Such differing use may require different HNCMARKER combinations in individual panel, mathematical algorithm, and/or cut-off points, but be subject to the same aforementioned measurements of accuracy and other performance metrics relevant for the intended use.

Identifying the subject who will be responsive to therapy enables the selection and initiation of various therapeutic interventions or treatment regimens in order increase the individuals survival potential. Levels of an effective amount of HNCMARKER proteins, nucleic acids, polymorphisms, metabolites, or other analytes also allows for the course of treatment of a metastatic disease or metastatic event to be monitored. In this method, a biological sample can be provided from a subject undergoing treatment regimens, e.g., drug treatments, for cancer. If desired, biological samples are obtained from the subject at various time points before, during, or after treatment.

In certain embodiments, the methods of the invention are capable of predicting survivability and/or survival time of a head and neck cancer diagnosed subject, wherein the subject is predicted to live 3 months, 6 months, 12 months, 1 year, 2, years, 3 years, 4 years, 5 years, 6 years, 7 years, 8 years, 9 years, 10 years, 15 years, 20 years, 30 years, 40 years, or 50 years from the date of diagnosis or date or initiating a therapeutic regimen for the treatment of head and neck cancer

By virtue of HNCMARKERs' being functionally active, by elucidating its function, subjects with high HNCMARKERs, for example, can be managed with agents/drugs that preferentially target such pathways.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like cancer or cancer progression, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein.

Levels of an effective amount of HNCMARKER proteins, nucleic acids, polymorphisms, metabolites, or other analytes can then be determined and compared to a reference value, e.g. a control subject or population whose therapeutic responsiveness is known or an index value or baseline value. The reference sample or index value or baseline value may be taken or derived from one or more subjects who have been exposed to the treatment, or may be taken or derived from one or more subjects who are at low risk of surviving the cancer, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for cancer or and subsequent treatment for cancer or a metastatic event to monitor the progress of the treatment. A reference value can also comprise a value derived from risk prediction algorithms or computed indices from population studies such as those disclosed herein.

The HNCMARKERS of the present invention can thus be used to generate a “reference HNCMARKER profile” of those subjects who would or would not be expected respond to cancer treatment. The HNCMARKERS disclosed herein can also be used to generate a “subject HNCMARKER profile” taken from subjects who are responsive cancer treatment. The subject HNCMARKER profiles can be compared to a reference HNCMARKER profile to diagnose or identify subjects at risk for developing resistance to chemotherapy, to monitor the progression of disease, as well as the rate of progression of disease, and to monitor the effectiveness of treatment modalities. The reference and subject HNCMARKER profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

Differences in the genetic makeup of subjects can result in differences in their relative abilities to metabolize various drugs, which may modulate the symptoms or risk factors of cancer or metastatic events. Subjects that have cancer, or at risk for developing cancer or a metastatic event can vary in age, ethnicity, and other parameters. Accordingly, use of the HNCMARKERS disclosed herein, both alone and together in combination with known genetic factors for drug metabolism, allow for a pre-determined level of predictability that a putative therapeutic or prophylactic to be tested in a selected subject will be suitable for treating or preventing cancer in the subject.

To identify therapeutic or drugs that are appropriate for a specific subject, a test sample from the subject can also be exposed to a therapeutic agent or a drug, or radiation, and the level of one or more of HNCMARKER proteins, nucleic acids, polymorphisms, metabolites or other analytes can be determined. The level of one or more HNCMARKERS can be compared to sample derived from the subject before and after treatment or exposure to a therapeutic agent or a drug, or can be compared to samples derived from one or more subjects who have shown improvements in risk factors (e.g., clinical parameters or traditional laboratory risk factors) as a result of such treatment or exposure.

A subject cell (i.e., a cell isolated from a subject) can be incubated in the presence of a candidate agent and the pattern of HNCMARKER expression in the test sample is measured and compared to a reference profile, e.g., a metastatic disease reference expression profile or a non-disease reference expression profile or an index value or baseline value. The test agent can be any compound or composition or combination thereof, including, dietary supplements. For example, the test agents are agents frequently used in cancer treatment regimens and are described herein.

The aforementioned methods of the invention can be used to evaluate or monitor the progression and/or improvement of subjects who have been diagnosed with a cancer, and who have undergone surgical interventions.

### Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic, predictive, or prognostic test, assay, or method concerns the ability of the test, assay, or method to distinguish between subjects responsive to chemotherapeutic treatment and those that are not, is based on whether the subjects have an “effective amount” or a “significant alteration” in the levels of a HNCMARKER. By “effective amount” or “significant alteration,” it is meant that the measurement of an appropriate number of HNCMARKERS (which may be one or more) is different than the predetermined cut-off point (or threshold value) for that HNCMARKER(S) and therefore indicates that the subject responsiveness to therapy or disease free/overall survival for which the HNCMARKER(S) is a determinant. The difference in the level of HNCMARKER between normal and abnormal is preferably statistically significant. As noted below, and without any limitation of the invention, achieving statistical significance, and thus the preferred analytical and clinical accuracy, generally but not always requires that combinations of several HNCMARKERS be used together in panels and combined with mathematical algorithms in order to achieve a statistically significant HNCMARKER index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining the clinically significant presence of HNCMARKERS in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.80, desirably at least 0.85, more desirably at least 0.875, preferably at least 0.90, more preferably at least 0.925, and most preferably at least 0.95.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for therapeutic unresponsiveness, and the bottom quartile comprising the group of subjects having the lowest relative risk for therapeutic unresponsiveness Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the HNCMARKERS of the invention allows for one of skill in the art to use the HNCMARKERS to identify, diagnose, or prognosis subjects with a pre-determined level of predictability and performance.

### Construction of Clinical Algorithms

Any formula may be used to combine HNCMARKER results into indices useful in the practice of the invention. As indicated above, and without limitation, such indices may indicate, among the various other indications, the probability, likelihood, absolute or relative chance of responding to chemotherapy or chemoradiotherapy. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from HNCMARKER results in the relevant training population. Amongst other uses, such formula may be intended to map the feature space derived from one or more HNCMARKER inputs to a set of subject classes (e.g. useful in predicting class membership of subjects as normal, responders and non-responders), to derive an estimation of a probability function of risk using a Bayesian approach (e.g. the risk of cancer or a metastatic event), or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual HNCMARKER measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derived using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

Combination with Clinical Parameters and Traditional Laboratory Risk Factors

Any of the aforementioned Clinical Parameters may be used in the practice of the invention as a HNCMARKER input to a formula or as a pre-selection criteria defining a relevant population to be measured using a particular HNCMARKER panel and formula. As noted above, Clinical Parameters may also be useful in the biomarker normalization and pre-processing, or in HNCMARKER selection, panel construction, formula type selection and derivation, and formula result post-processing. A similar approach can be taken with the Traditional Laboratory Risk Factors, as either an input to a formula or as a pre-selection criteria.

### Measurement of HNCMARKER

The actual measurement of levels or amounts of the HNCMARKERS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of HNCMARKERS can be measured using reverse-transcription-based PCR assays (RTPCR), e.g., using primers specific for the differentially expressed sequence of genes or by branch-chain RNA amplification and detection methods by Panomics, Inc. Amounts of HNCMARKERS can also be determined at the protein level, e.g., by measuring the levels of peptides encoded by the gene products described herein, or subcellular localization or activities thereof using technological platform such as for example AQUA. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The HNCMARKER proteins, polypeptides, mutations, and polymorphisms thereof can be detected in any suitable manner, but is typically detected by contacting a sample from the subject with an antibody which binds the HNCMARKER protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (e.g., anti-HNCMARKER protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, quantum dots, multiplex fluorochromes, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., 35S, 125I, 131I), enzyme labels (e.g., horseradish peroxidase, alkaline phosphatase), and fluorescent labels (e.g., fluorescein, Alexa, green fluorescent protein, rhodamine) in accordance with known techniques. Highly sensitivity antibody detection strategies may be used that allow for evaluation of the antigen-antibody binding in a non-amplified configuration. In addition, antibodies may be conjugated to oligonucleotides, and followed by Polymerase Chain Reaction and a variety of oligonucleotide detection methods.

Antibodies can also be useful for detecting post-translational modifications of HNCMARKER proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization time of flight mass spectrometry (MALDI-TOF) (Wirth, U. et al. (2002) Proteomics 2(10): 1445-51). In addition to post-translation modifications, these processes may be coupled to localization of the protein, such that a re-localization process is monitored, and the biomarker is evaluated in a relative fashion exhibited by the constancy or change to the ratio of the protein in different compartments. Important to several of the proteins in HNCMARKERs, nuclear, nuclear foci, and cytoplasmic sites in tumor cells are evident.

For HNCMARKER proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Using sequence information provided by the database entries for the HNCMARKER sequences, expression of the HNCMARKER sequences can be detected (if present) and measured using techniques well known to one of ordinary skill in the art. For example, sequences within the sequence database entries corresponding to HNCMARKER sequences, or within the sequences disclosed herein, can be used to construct probes for detecting HNCMARKER RNA sequences in, e.g., Northern blot hybridization analyses or methods which specifically, and, preferably, quantitatively amplify specific nucleic acid sequences. As another example, the sequences can be used to construct primers for specifically amplifying the HNCMARKER sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

Alternatively, HNCMARKER protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in their entireties) In this regard, other HNCMARKER analytes can be measured using the above-mentioned detection methods, or other methods known to the skilled artisan. For example, circulating calcium ions (Ca2+) can be detected in a sample using fluorescent dyes such as the Fluo series, Fura-2A, Rhod-2, among others. Other HNCMARKER metabolites can be similarly detected using reagents that are specifically designed or tailored to detect such metabolites.

### Kits

The invention also includes a HNCMARKER-detection reagent, e.g., nucleic acids that specifically identify one or more HNCMARKER nucleic acids by having homologous nucleic acid sequences, such as oligonucleotide sequences, complementary to a portion of the HNCMARKER nucleic acids or antibodies to proteins encoded by the HNCMARKER nucleic acids packaged together in the form of a kit. The oligonucleotides can be fragments of the HNCMARKER genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, HNCMARKER detection reagents can be immobilized on a solid matrix such as a porous strip to form at least one HNCMARKER detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of HNCMARKERS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by HNCMARKERS. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

Suitable sources for antibodies for the detection of HNCMARKERS include commercially available sources such as, for example, Abazyme, Abnova, Affinity Biologicals, AntibodyShop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Ab Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, nucleic acid probes, e.g., oligonucleotides, aptamers, siRNAs, antisense oligonucleotides, against any of the HNCMARKERS disclosed herein.

### EXAMPLES

### Example 1

### General Methods of Evaluating Head and Neck Cancer Patients with Biomarkers

### Patient Cohorts

### A. Induction Chemotherapy

Squamous cell carcinomas of the head and neck are highly responsive to induction chemotherapy. Head and neck cancer patients with stage III and IV locoregionally advanced HNSCC received carboplatin/taxane (C+T) induction chemotherapy followed by FHX based chemoradiotherapy [(paclitaxel, 5-Fluorouracil, hydroxyurea)] according to the approved protocols from a collaborating cancer center. However, chemotherapy is toxic to patients and it is preferred to understand the benefit of the treatment by an evaluation of molecular biomarkers of the tumor cells prior to treatment.

Response evaluation was performed after induction chemotherapy. Response criteria were based on two dimensional tumor measurements. Complete response (CR) was defined as complete disappearance of all detectable disease. Attempts to document complete remission by biopsy or surgery of previously involved areas were made. Partial response (PR) was defined as reduction by at least 50% of the products of the longest perpendicular diameters of measurable tumor lesions, with no growth of other lesions and no appearance of new lesions. Stable disease (SD) was defined by the same criteria as partial response except that tumor lesions remained stable in size or decreased by less than 50%. Progressive disease (PD) was defined as gross disease enlargement, an increase of >=25% of the product of perpendicular diameters of tumor lesions or the appearance of new metastatic lesions. The response rate was expressed as the proportion of patients who demonstrated complete and/or partial response. Best response was defined as the best response clinically or radiographically. Patients with >=N2 disease were recommended to undergo post-therapy selective neck dissection. Salvage surgery was recommended for residual disease after chemoradiotherapy. Surgery at the primary was omitted for patients with a CR confirmed by post-therapy physical examination, radiographic imaging, and/or negative biopsy (Vokes, E E et al 2000, Kies M S et al 2001, Salama J K et al 2008).

Thirty-seven locoregionally advanced HNC patients were studied where the tumor samples had been formalin-fixed paraffin embedded (FFPE). All patients had been treated with induction chemotherapy consisting of 2 cycles of paclitaxel and carboplatin for a total of eight weeks and then evaluated for response. Of the 37 HNC patients in the study, 11 had complete response (CR), 19 had partial response (PR), and 7 had stable disease (SD). The goal of the study was to develop a biomarker pattern at the biopsy stage that would predict the response of patients to induction chemotherapy.

### B. Concurrent Chemoradiotherapy

In a later phase of treatment of Head and Neck cancer patients, there is frequently the use of radiation and chemotherapy, termed concurrent chemoradiotherapy. Patients may be candidates for concurrent chemoradiotherapy at any stage of the cancer (I-IV), and may or may not have previously received surgery. In addition, patients having a tumor recurrence are also candidates for concurrent chemoradiotherapy and/or a clinical decision about whether to receive re-irradiation. In each of these circumstances, there is not adequate molecular profiling of the patient's tumor cells to discriminate whether they are expected to benefit from the treatment.

Biopsy specimens (paraffin embedded tumor samples) from 66 locoregionally advanced HNC patients in the collaborating cancer center were evaluated from tissue microarrays. The HNC patient biopsies had been obtained from a primary excision or recurrent biopsy. Samples are from phase I/II studies: 1) poor-prognosis radiation-naïve, 2) re-irradiation. All were treated with TFHX-based chemoradiotherapy [(paclitaxel, 5-Fluorouracil, hydroxyurea)]. Patients received chemotherapy [(paclitaxel, 5-Fluorouracil, hydroxyurea)] and radiation for 5-7 cycles which is total 10-14 weeks, each chemotherapy and radiation cycle was given for 5 days.

Patients having received concurrent chemoradiotherapy are evaluated by a number of clinical criteria. The determination of biomarkers indicating the prognostic and predictive benefit of the treatment is weighed against these clinical parameters. For Head and Neck Cancer, patients are monitored for cancer-related events such as a recurrence, distant metastasis, or death. Evaluable clinical indicators are Overall Surivival, Disease-Free or Progression-Free Survival, also Time to Progression or Time to Event. Patients may be subgrouped or classified by the recurrence type, such as an event associated with a cancer other than Head and Neck cancer, or by a second cancer of the head and neck.

C. Patients that are HPV-Positive or HPV-Negative with Head and Neck Cancers

Although tobacco and alcohol consumption are the primary risk factors for development of Head and Neck Cancer, high-risk human papillomavirus (HPV), commonly HPV16, is an emerging etiologic factor in Head and Neck cancer. Nonsmokers with oropharynx carcinomas are 15-fold more likely to be HPV positive than smokers. Recent studies have indicated that in Head and Neck Cancer, patients with HPV-infected tumors have a more favorable prognosis compared with patients whose tumors are virus-negative (Fakhry C et al 2008, Licitra L et al 2006, Weinberger P M et al, 2006, Kumar B et al 2008). It is also noted that patients with HPV-positive oropharynx tumors have a survival advantage, HPV copy number per cell was significantly associated with a better response to induction chemotherapy and concurrent chemoradiation as well as with improved disease specific survival and overall survival (Worden F P et al 2008). The p16 protein (p16) is a cyclin-dependent kinase (CDK) inhibitor that inhibits retinoblastoma (Rb) phosphorylation and blocks cell cycle progression at the G1 to S checkpoint. HPV-positive tumors are characterized by high expression of p16. Functional inactivation of pRb by HPV E7 protein results in overexpression of p16, which makes it a surrogate marker for HPV. (Fakhry C et al 2008, Licitra L et al 2006, Weinberger P M et al, 2006). Thus there is good evidence that p16 positivity may be regarded as a biomarker for tumors harboring clinically and oncogenetically relevant HPV infections. The combined p16/HPV biomarker data are associated with different survival outcomes of HNC compared to each marker evaluated separately, indicating that the two molecular mechanisms evaluated together may provide a more accurate prediction of clinical outcomes (Smith E M et al 2008).

### Biomarker Assays by Use of Antibody-Based Immunohistochemistry

The whole sections of tumors or Tissue Microarrays (TMAs) of the tumor specimen cores were stained by immunohistochemistry (IHC) using antibodies against proteins in DNA repair pathways (Table 1). The proteins or epitopes that are HNCMarkers include XPF and ERCC1 (nucleotide excision repair), FANCD2 (Fanconi Anemia/homologous recombination pathway), MLH1 (mismatch repair), PARP1 (base excision repair), PAR (poly-ADP ribose, base excision repair), pMK2 (phospho-MAPKAP Kinase2, DNA damage response), pHSP27 (DNA damage response), ATM (DNA damage response), pH2AX (DNA damage response/non-homologous end joining), ERCC1 (nucleotide excision repair), p53 (DNA damage response), RAD51 (homologous recombination), Ki67 (proliferation marker). The antibodies were obtained from the following sources: XPF and ERCC1 (AbCam), ATM (Epitomics), FANCD2 and p53 (Santa Cruz), MLH1 and Ki67 (BioCare Medical), PARP1 (AbD Serotec), PAR (polyADP ribose) and pH2AX and RAD51 (Millipore), phospho-MAPKAP Kinase2 and pHSP27 (Cell Signaling Technology).

HPV status is monitored by a variety of detection methods, including DNA, RNA, and protein based tests for the viral genome, transcripts, or proteins. HPV in situ hybridization (ISH) assays using paraffin embedded tumor samples were performed. ISH was performed using (Ventana INFORM HPVII and HPVIII kit) which contain HPV high risk and low risk DNA probes linked to a chromagen-based detection strategy. In addition, surrogate biomarkers of HPV infection are also used. P16, the tumor suppressor, is upregulated and stabilized in HPV-infected cells. p16 is monitored by IHC as for DNA repair biomarkers.

In addition to the head and neck cancer specimens in the study group, IHC was conducted with negative and positive human head and neck cancer control sections. Tissue sections were deparaffinized and rehydrated using standard techniques. Heat-induced epitope retrieval was performed and the tissues were stained with antibody overnight at 4° C. Renaissance TSA™ (Tyramide Signal Amplification) Biotin System (Perkin Elmer) was used for detection of XPF and FANCD2. Super Sensitive™ IHC Detection System (BioGenex) was used for detection of MLH1, PARP1, PAR, pMK2, pHSP27, pH2AX, ERCC1 and Ki67. Envision+ System-HRP (Dako) was used for detection of p53, ATM, RAD51. Two-fold antibody dilution ranges were established, and antigen retrieval conditions were set such that antibody was in excess and discriminated between control cancer tissues between low and high expression levels.

### Change of Expression of DNA Repair Protein is Observed Frequently in Head and Neck Cancer

DNA repair pathways are important to the cellular response network to chemotherapy and radiation. Representatives from several of these DNA repair pathways were investigated for associations with clinical outcome in induction chemotherapy study (Table 1). The HNC patient biopsies had been obtained from a primary excision or recurrent biopsy. Thirty-seven whole tissue sections of HNC patient specimens were applied to immunohistochemistry. Ten selected DNA repair protein epitopes, Ki67, and several other biomarkers were evaluated in serial sections from head and neck cancer specimens. Tumor zones were demarcated per section by pathology review. Expression differences for the markers were quantified by scanning microscope slides into a digital pathology platform (Aperio). Pathologist' scores and machine-based collection of staining intensities was concentrated to the annotated tumor zones. Marker outputs in 0, 1+, 2+, and 3+ bins were combined in a weighting algorithm to create a relative intensity score from 0-300. For several markers, the intensity of nuclear staining was gauged, in other cases, localization of the marker into different cell compartments was revealed. For example, the nuclear staining pattern of the nucleotide excision repair (NER) protein biomarker XPF can be demonstrated in three representative cancer tissue biopsies by immunohistochemistry. Low or negative intensity of XPF nuclear staining indicates that NER pathway is off, and medium or high intensity of XPF staining indicates that NER pathway is on. Image analysis algorithms were established for each biomarker with control Head and neck tumor sections. Likewise, post-translational regulation was demonstrated selectively in a tumor-specific manner with several other antibodies in the group. For example, by monitoring the phosphorylation modification of Mapkapkinase2 (pMK2) or pATM or pH2AX, selective changes between patient specimens were discriminated. In addition, subcellular localization of pMK2 occurred in several distributions including nuclear only, or nuclear+cytoplasmic depending on the patient tumor. Several biomarkers such as FANCD2 or BRCA1 proteins have a distinctive pattern in the nucleus of cells that are indicators of change in activation of the DNA repair pathway. For these proteins, IHC or immunofluorescence-based nuclear foci are indicative of activation of the FA/HR pathway. For head and neck tumor biopsies, it is evident that certain patient specimens show activation of FANCD2 and BRCA1-based DNA repair pathways, and other specimens do not.

### Scoring

IHC quantitative comparisons are established by digital pathology strategies involving the conversion of a microscope slide chromagen staining pattern to a computer-based image, and the utilization, adaptation, and training of software algorithms. The stained tissue was evaluated using machine-based image analysis and scoring that incorporated the intensity and quantity of positive tumor nuclei. Scanning and image analysis platforms were from Aperio. Each biomarker pattern was assessed for quality and by pathology overview. Image analysis algorithms were established for each biomarker with control head and neck cancer tumor sections.

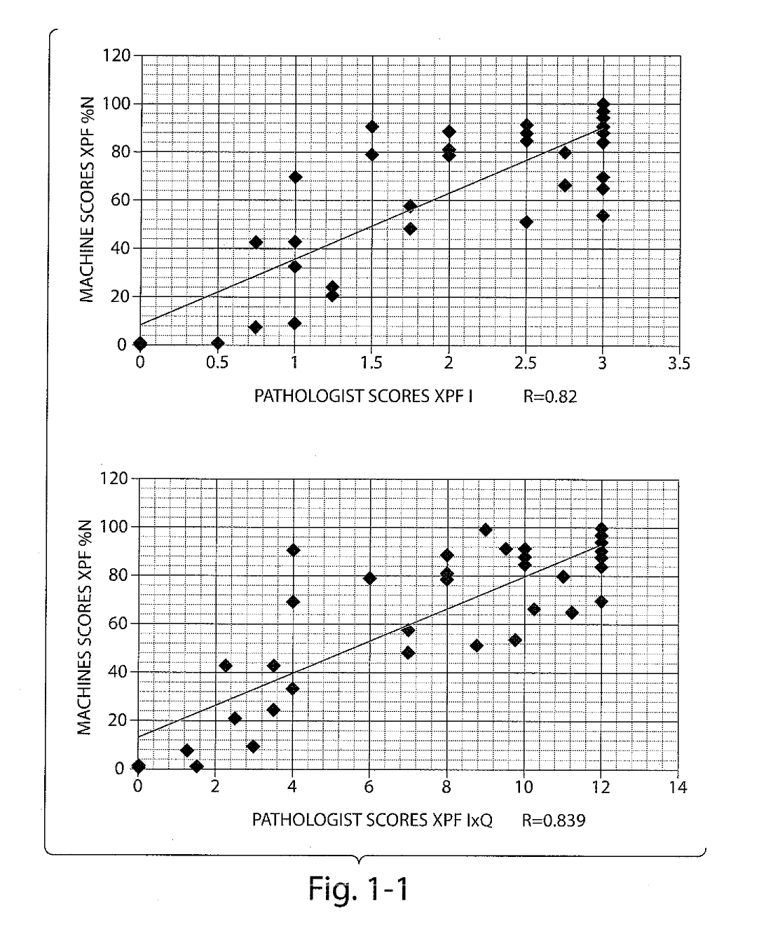

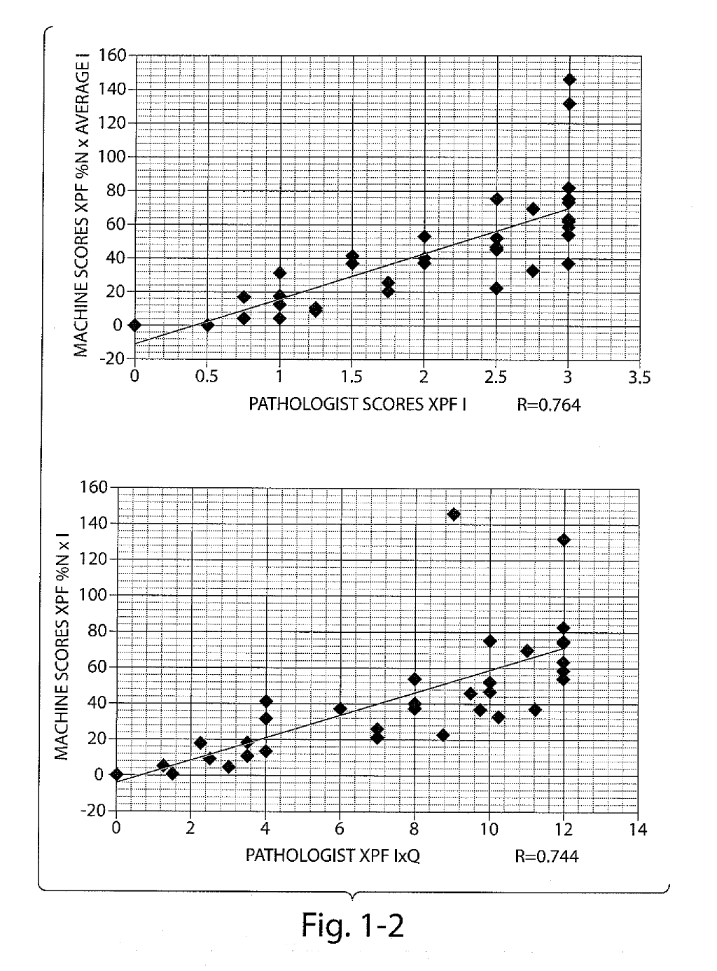

To test the correlations between machine-guided image analysis and pathologist scores, we compared IHC stained XPF which were analyzed by two blinded pathologists and machine-based algorithm in the induction chemotherapy study (FIG. 1). The results showed good correlations between machine-guided image analysis and pathologist scores with R² value range from 0.744-0.839.

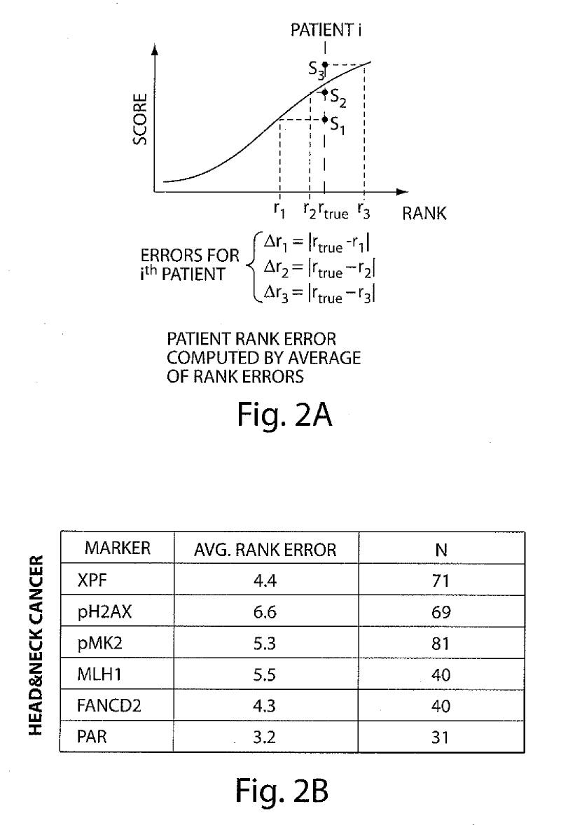

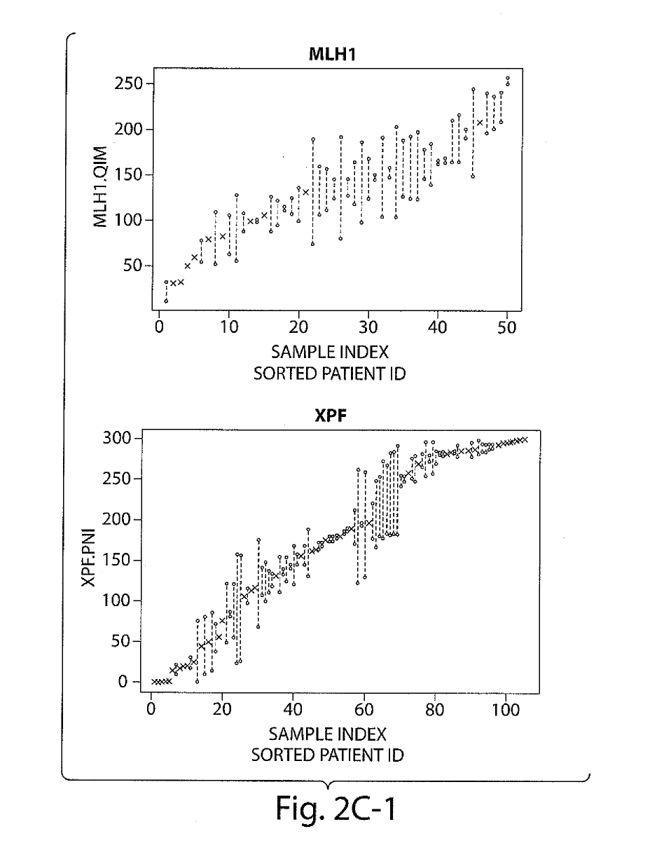

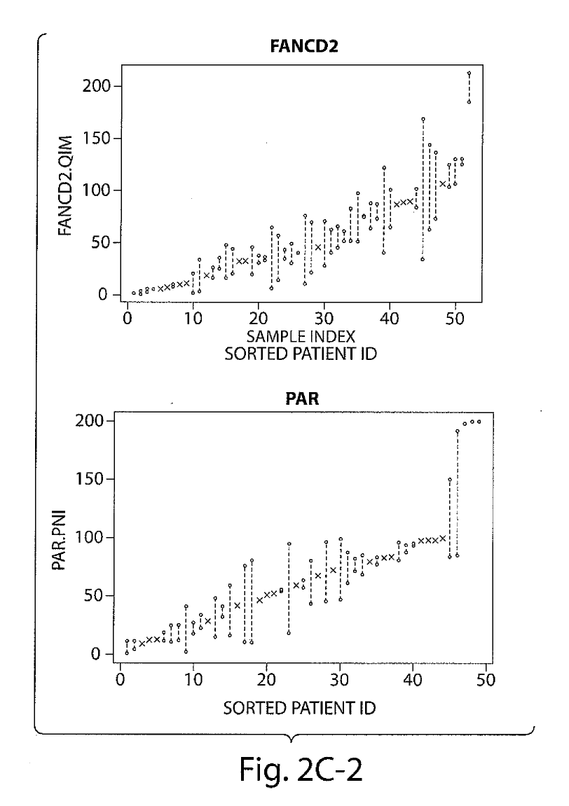

To discriminate the marker output values relative to clinical outcome correlates, it was sought first to resolve whether specimen core-core variation influenced a patient ranking scheme for DNA repair markers. For this purpose, an arbitrary index of patient ranks was established from the lowest values in the cohort to the highest values. The level of variation of each of the markers between triplicate TMA cores was determined, and scored against the patient rank value/marker (FIG. 2). For the eight HNCMARKERS tested, it was found that the average rank error was a low percentage of the total (8.8-11.1% DNA Repair, 11.1% Ki67). Therefore, relatively minor variations between duplicate TMA cores do not significantly change the patient rank order for any of the markers tested.

### Statistical Analysis

Biomarker scoring was correlated with clinical data to assess for correlation with outcome. A set of optimal threshold marker values were determined by univariate analysis for each marker that yielded the highest discrimination between responder and non-responder groups (Induction chemotherapy) or to separate good survival and poor survival groups (Concurrent chemoradiotherapy). Discriminant and partition analysis was also conducted to maximally separate the dataset samples into groups for responders/non-responder or by disease-free or overall survival. The biomarkers were chosen from different DNA repair pathways so that algorithms using multiple markers would capture information on multiple pathways instead of redundant measurements of the same pathway. Kaplan-Meier survival curves and Cox proportional hazards were used to evaluate time to death associations with biomarkers individually or in combination. Statistical outputs for p-value, Apparent Error Rate (AER), Receiver Operator Characteristics and Area Under Curve (AUC), Sensitivity, Specificity, Positive Predictive Power, Negative Predictive Power, Relative Risk (RR), Odds Ratio were computed in the alternative models. With multi-marker models, a separate analysis involving probability tests were also conducted to produce AUC and other statistical values.

### Example 2

### HNCMARKERS that have Utility in Discriminating Benefit from Concurrent Chemoradiotherapy

Sixty-six HNC patients with stage III and IV locoregionally advanced HNSCC received TFHX-based concurrent chemoradiotherapy according to the approved protocols at the collaborating cancer center. The patient biopsies had been obtained from a primary excision or recurrent biopsy and three Tissue Microarrays (TMAs) were constructed and applied in immunohistochemistry biomarker development. Time to progression was measured as the time from the first day of therapy until death of disease, appearance of new lesions, or a greater than 25% increase of the indicator lesion over the previous smallest size. Survival was measured from the date of entry onto the study until death of any cause. Disease-free survival (DFS), and overall survival (OS) and Disease-Specific Survival (DSS) were calculated from the date of initial diagnosis. DFS was defined as the time between tissue acquisition and evidence of disease recurrence. OS was defined as the time from randomization to death from any cause (Yokes E E et al 2000, Michiels S et al 2009). For purposes of statistical analysis local, regional or distant recurrence were grouped together as disease recurrence. Time to progression and survival time were summarized using Kaplan-Meier product limit curves.

The goal of the study was to develop a biomarker pattern at the biopsy stage that would predict the rate of disease-free or overall survival, and inform how aggressively a patient's tumor would return under Concurrent chemoradiotherapy. Several examples of the utility of these biomarkers in this clinical setting are illustrated.

### Example 3

### Association of HNCMARKERS by Partition Analysis with Separation of Head and Neck Cancer Patient Survival Groups Following Concurrent Chemoradiotherapy

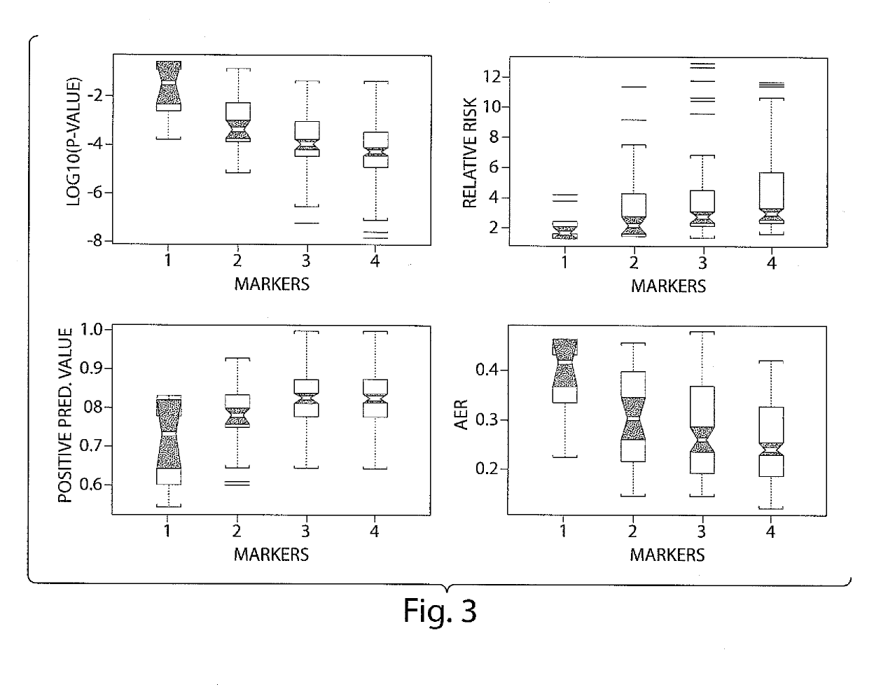

Eleven HNCMARKERS were analyzed from biopsy material for their ability to predict the likelihood of survival following the concurrent chemoradiotherapy (Table 1). Each HNCMARKER were then evaluated for the separation between death and disease-free/overall survival groups. Univariate Cox proportional hazards models were constructed for each of the markers (single marker models) to examine their potential predictive powers. High XPF (p=0.00422), FANCD2 (p=0.00199), RAD51 (p=0.0359), and BRCA1 (p=0.0101) were associated with better survival on univariate partition analysis (Table 2). Kaplan-Meier survival curves also show that high XPF, FANCD2, BRCA1, RAD51, ATM were associated with better survival outcome, which consistent with univariate partition analysis (FIG. 3). For several other markers in DNA repair such as pMK2 and pH2AX, ERCC1, PAR, p53, the same analysis failed to reach statistical significance (Table 2).

Discovery of Multiple DNA Repair Biomarker Panels that Distinguish Survival Groups

The DNA repair pathways may operate in cell survival and chemotherapy responses in a concerted way. Therefore, DNA repair proteins changes may be more effectively determined by combining the effects of markers, rather than by individual analysis. In order to develop a statistically-driven hypothesis for these associations, the combination of multimarkers were analyzed using distributive partitioning. Models consisting of combinations of markers were constructed to investigate possible complimentary interactions between markers and pathways for separation of death and survival groups.

Two marker model partition analyses were separately calculated for concurrent chemoradiotherapy association of pairs of biomarkers (Table 3). XPF, FANCD2, BRCA1, RAD51, ATM appeared in 78% of the two marker partition and probability models that have lower P values and higher AUC values. FANCD2, BRCA1, RAD51, ATM in two marker analyses show better separation of survival group from head and neck patients. Other markers did not perform consistently in similar pairwise tests, they were not observed to belong to another group, and were not found to contribute to greater discrimination of survival groups. All two marker partition models were computed for the HNCMARKERS (Table 3). Statistical evaluation included p value, Apparent Error Rate, Relative Risk, Odds Ratio, Positive predictive power, and Negative predictive power.

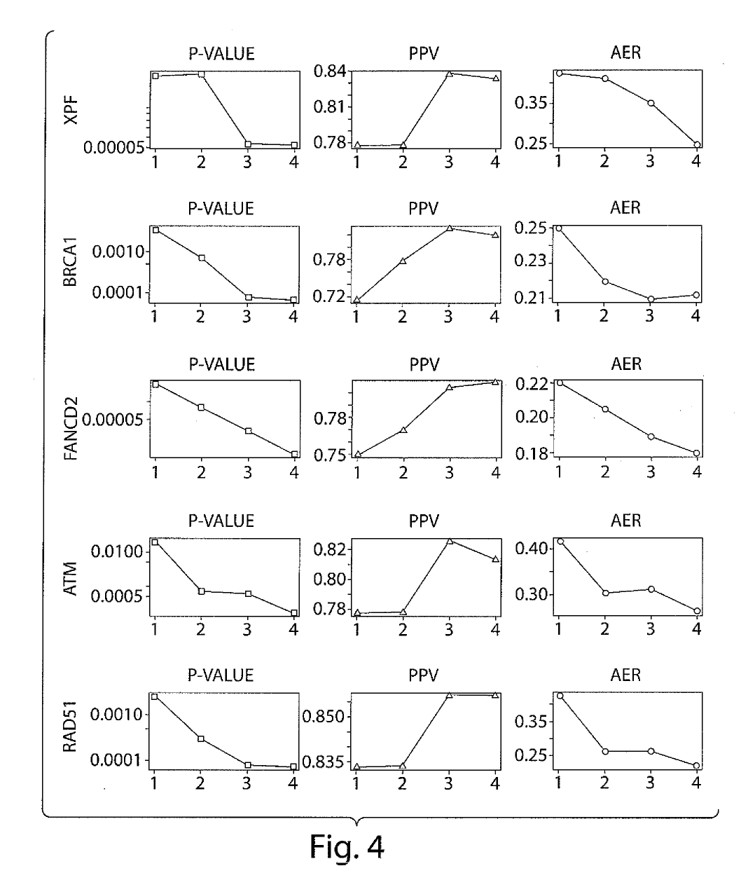

HNCMARKERS in combination are more effective than separately, and identifying a root marker with high performance in marker combinations is important. A platform was developed to analyze the capabilities of individual markers when used in combinations with others. The tests were run by evaluating the root marker performance improved in multimarker models for overall survival following treatment with concurrent chemoradiotherapy. The role of this analysis is to associate specific marker groups for subsequent testing in additional models. A partition analysis was calculated for the HNCMARKERS in the study with targeted start points of specific single biomarkers. In the example shown (FIG. 4), there are five root markers, FANCD2, XPF, BRCA1, ATM and RAD51 that were calculated. Shown are the starting 1-marker models and then all 2-, 3-, and 4-marker models that always will contain the root marker. In each case, the computed log 10 P-value (squares), Positive Predictive Value (PPV) (triangles) and AER (black circles) are shown for each Root Marker alone, and in combination with other HNCMARKERS in 2-, 3-, and 4-marker models. The median values of all the models are plotted for each model.

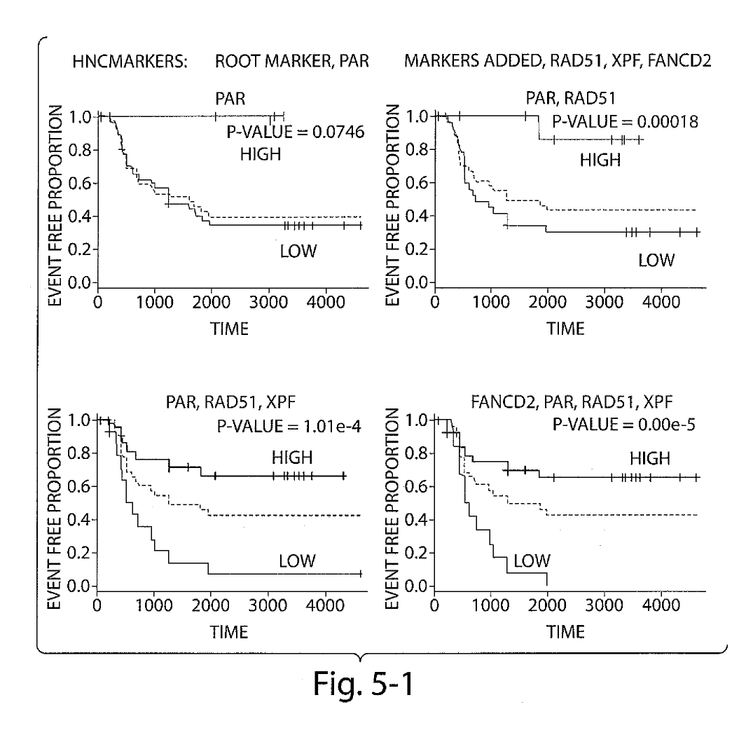

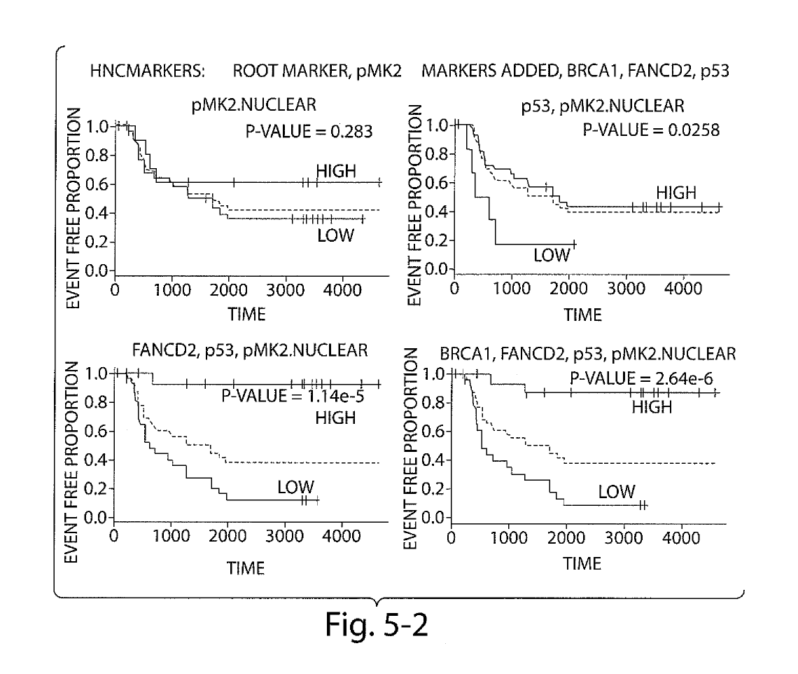

An example of the benefit of marker addition to important root markers is demonstrated in this exercise. Two specific examples from the Root marker analysis were chosen, PAR and pMK2, and Kaplan-Meier Overall Survival curves were plotted with p-values calculated to indicate the discrimination between High Survival and Low Survival patient subgroups. In the first example with PAR, the HNCMARKERS RAD51, XPF, and FANCD2 were added and the respective 2-, 3-, and 4-marker models calculated (FIG. 5). The p-values were PAR (0.0746), PAR, RAD51 (0.00618), PAR, RAD51, XPF (1.61e-4), and PAR, RAD51, XPF, FANCD2 (6.06e-5) indicating that addition of markers (markers in combination) better discriminates patient survival subgroups. In the second example with pMK2, the HNCMARKERS p53, FANCD2, and BRCA1 were added, and the respective 2-, 3-, and 4-marker models calculated (FIG. 5). The p-values were pMK2 (0.293), pMK2, p53 (0.0256), pMK2, p53, FANCD2 (1.1e-5), and pMK2, p53, FANCD2, BRCA1 (2.64e-6). In both examples, All Patients in the study are demarcated by a black dashed line, and the 4-marker models illustrated identify patient subgroups distinct from the All Patients trend.

Multi-marker partition models were further extended to include combinations of three markers, and four markers. Three marker partition models were computed for the HNCMARKERS and the lists prioritized by statistical values (Table 4). In one example in Kaplan-Meier OS curves indicates that high RAD51, BRCA1 and FANCD2 were associated with better survival outcome with P value=1.22e-4, AUC=0.84 as compared with single marker models (Table 2). Furthermore, all the three marker models were jointly analyzed in Overall Survival analysis distinguishing good and poor survival subgroups following concurrent chemoradiotherapy.

To demonstrate that multi-HNCMARKERS show improved performance over single markers, the partition analysis output was evaluated against the six statistical values and a comparison of the 1-, 2-, 3-, and 4-marker models with the group of HNCMARKERS in concurrent chemoradiotherapy study (Tables 2-5). The results indicate that based on the values of P value, Relative Risk, Positive Predictive Value, Specificity, and AER, that increasing the number of markers from this group in the model leads to an increased performance where 3-, 4-, and 5-marker models are clearly superior and non-overlapping with the 1-marker models. Therefore, both four HNCMARKER tests and five HNCMARKER tests give better discrimination and fewer errors than a single HNCMARKER.

### Example 4

### Use of a Parametric Probability Analysis on HNCMARKERS to Effectively Distinguish Good Survival and Poor Survival Groups with Head and Neck Cancers

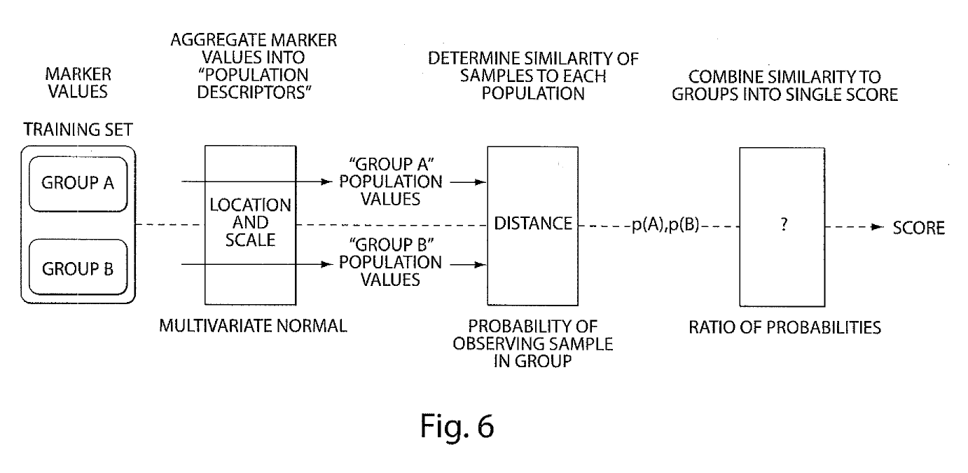

A probability analysis statistical process was independently executed to compare the HNCMARKERS in concurrent chemoradiotherapy study. A procedure was developed to examine the placement of a patient in a death or survival group by examining the probability of observing the marker evaluation in each group (FIG. 6). In this procedure, we refine the definition of group membership used in the above analysis by defining a region of low incidence of death in addition to the region of high incidence of death. These regions are constructed using multivariate probability distributions for the likely to die and not likely to die groups and a single score reflecting group membership is constructed from the individual group probabilities. One method of constructing these probability distributions is to use a parametric estimation of the probabilities, i.e. normal distributions. Another method is to use a non-parametric (distribution free) estimate of the probability densities for each group.

### Theory and Calculations of Parametric Method (Normal Distribution):

By measuring a mean vector, μ, and covariance matrix, Σ, for both groups, the probability density function can be evaluated for the not likely to recur, fₙₗ(x), and the likely to recur, fₗ(x), groups given the marker values, x.

$$\text{?} = ( \frac{1}{\text{?}} ) \text{?} ⌊ - \frac{1}{2} \text{?} ⌋$$

$$\text{?} \text{indicates text missing or illegible when filed}$$

The probability densities are expressed as a posterior probability of observing the marker values in each group.

$$P ( \text{?} ) = \text{?} and P ( \text{?} ) = \text{?}$$

$$\text{?} \text{indicates text missing or illegible when filed}$$

In order to obtain a scalar value to simplify interpretation these probabilities are combined into a score, s, via

$$s ( x ) = \frac{P ( nl ) - P ( n )}{P ( nl ) + P ( l )}$$

This form for the score is chosen so that a sample with much higher probability of being observed in the not likely to survive group (P(nl)>>P(l)) has a score close to +1; when the probability of being observed in the likely to survive group is much higher the score is close to −1. If the sample has nearly equal probability of being observed in both groups the score is close to zero. In order to accommodate samples where the outcome is unclear from the model, the magnitude of the score must exceed a threshold of ±⅓ before assigning to a group. A score of ±⅓ is equivalent to a 2-fold difference in group membership probability: P(nl)=2*P(l) or 2*P(nl)=P(l). If a sample does not exceed the threshold values, it is assigned to neither group and classed as indeterminate.

The mean and covariance matrices for each group are calculated from the dataset and are used to generate scores for a validation set.

Models using all unique combinations of one, two, three, and four markers were constructed and checked for their ability to discriminate patient's outcome. The number of samples that was indeterminate is plotted for all models. The median number of samples that fall in the indeterminate range (−⅓<score<⅓) decreases as more markers are added to the model. Outputs were evaluated in four ways: 1) Scores by Outcomes, 2) Kaplan-Meier survival Curve, 3) Predicted Outcome from Score, and 4) ROC Plot from Score. Scores are probabilities of an Event (Death) or No Event (Survival) and thus range from −1 to 1. Also, the Likelihood of an Event is also set to range between 0 and 1.

Scores by Outcomes indicates the likelihood of recurrence for a patient given their score. Likelihood of survival is plotted on the y-axis. A patient's survival likelihood is determined by reading the y-value from the curve corresponding to the x value (score). The indeterminate region, as defined above, is reflected in the plotting strategy as indicated by dashed lines and is (−⅓<score<⅓).

Predicted Outcome from Score is an assessment of the clinical relevance of the score by computing the likelihood of survival given a score value. The probability of recurrence for each level of score is calculated by binning all the patients within a score window (i.e. −1≦score≦0.8) and determining the percentage of patient samples within the window experiencing recurrence. Bins where the number of samples is less than 2 are not reported. The trend of the probability of recurrence vs. score is approximated using a Loess fit and the point-wise 95% confidence interval for the trend line is also reported (dotted lines in figures).

In addition, the ROC Plot from Score was used a determination of the quality of the test. The choice of ±⅓ for the indeterminate score threshold may not be optimal. The effect of choosing different score thresholds in assigning group membership can be examined using a ROC plot. A ROC plot is constructed from the score by moving a threshold from −1 to 1 and calling all samples less than the threshold positive for death or likely to die. All samples with scores greater than the threshold are allocated to the not likely to die group. The percentage of all recurrent samples correctly detected is plotted against the percentage of non-recurrent samples incorrectly identified as survival.

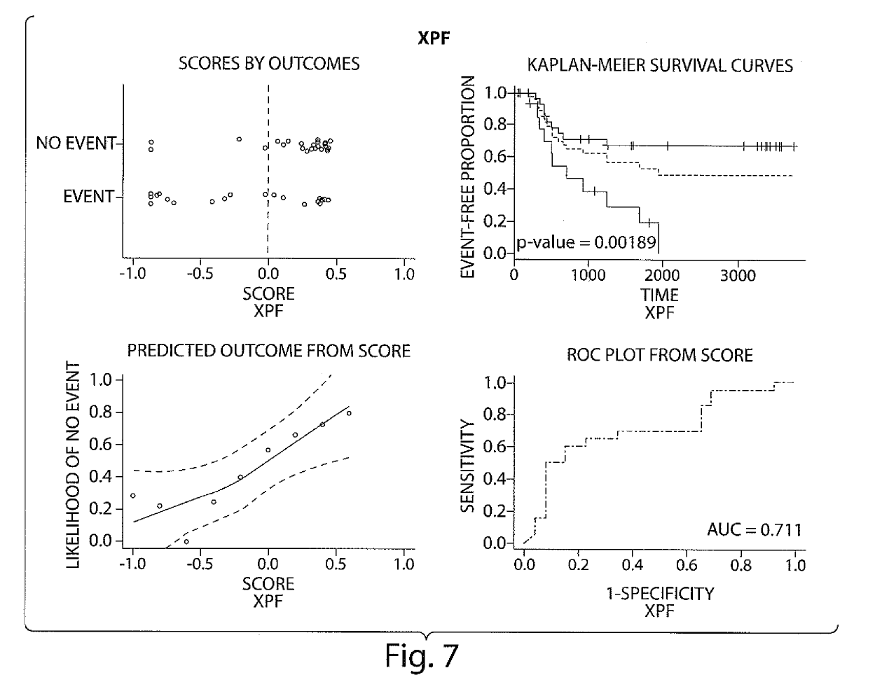

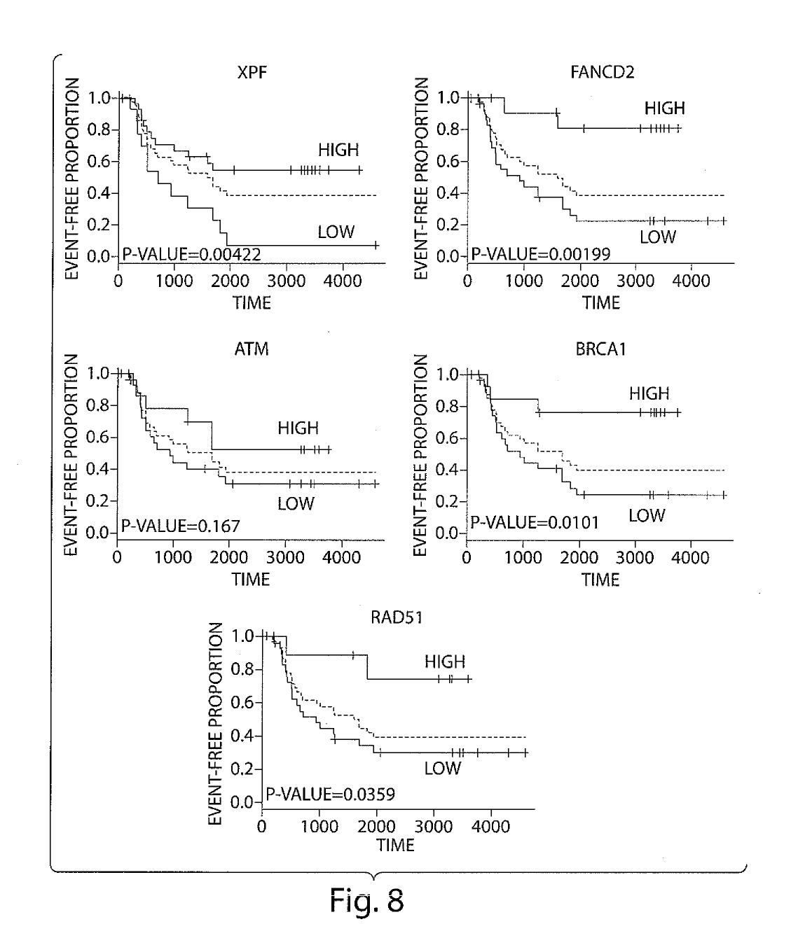

Using the above statistical parameters, HNCMARKERS were evaluated for associating with an overall survival benefit in Head and Neck cancer patients treated with concurrent chemoradiotherapy. An example HNCMARKER, XPF, is shown indicating the projections for the Scores by Outcomes, Kaplan-Meier disease-specific survival Curve, Predicted Outcome from Score, ROC Plot from Score (FIG. 7). In this example, the log 10 p-value distinguishing survival groups is 0.00189 (Kaplan-Meier Disease-specific survival curve), and the calculated AUC from the ROC plot is 0.711. A representative subgroup of the HNCMARKERS [XPF, FANCD2, ATM, BRCA1, and RAD51] Kaplan-Meier Overall Survival curves were also plotted from the outcome data for Head and Neck cancer patients treated with concurrent chemoradiotherapy (FIG. 8).

A Probability Analysis was also conducted for multiple HNCMARKERS with two marker models (Table 7), three marker models (Table 8), and four marker models (Table 9).

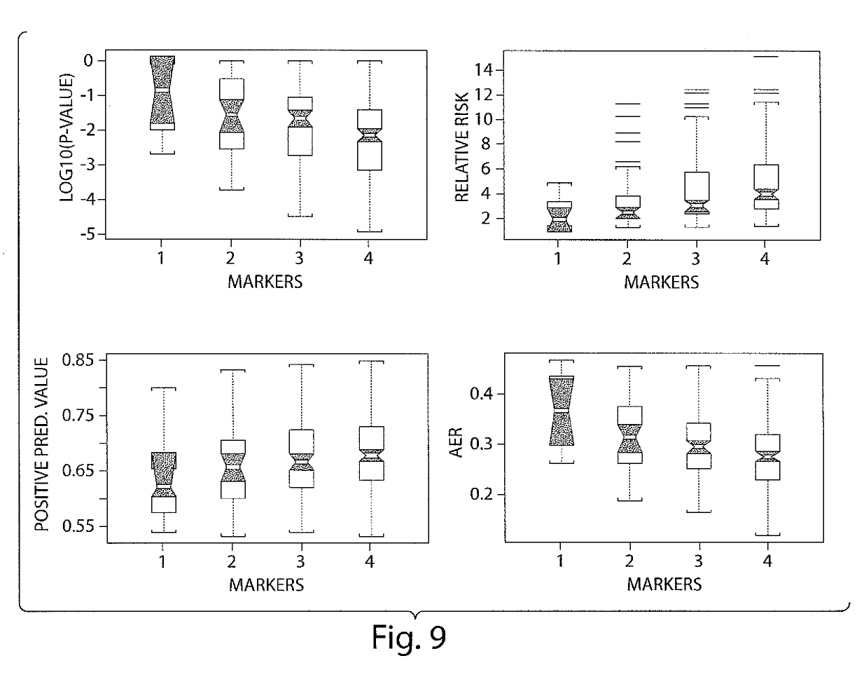

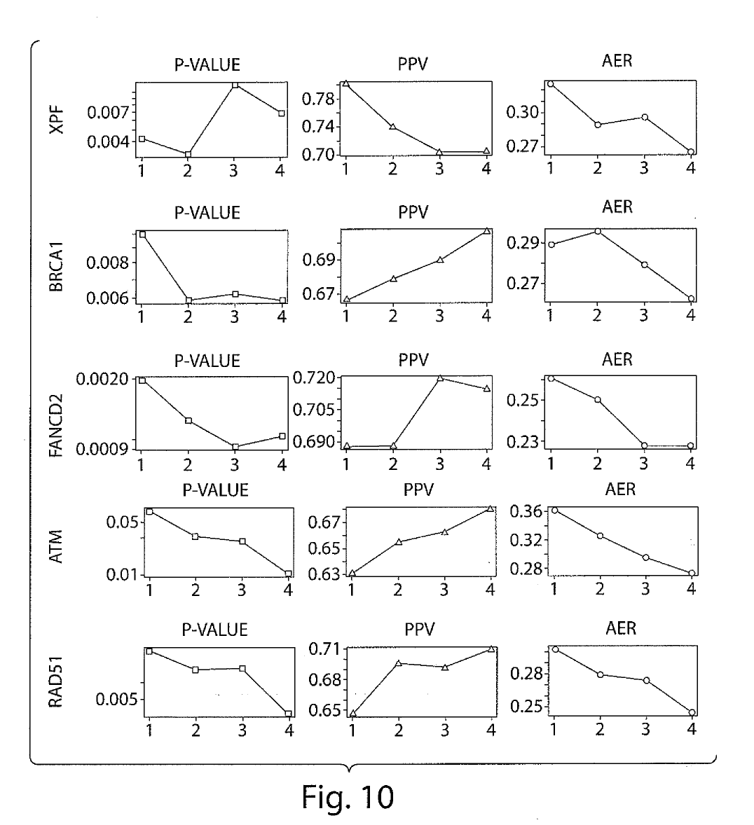

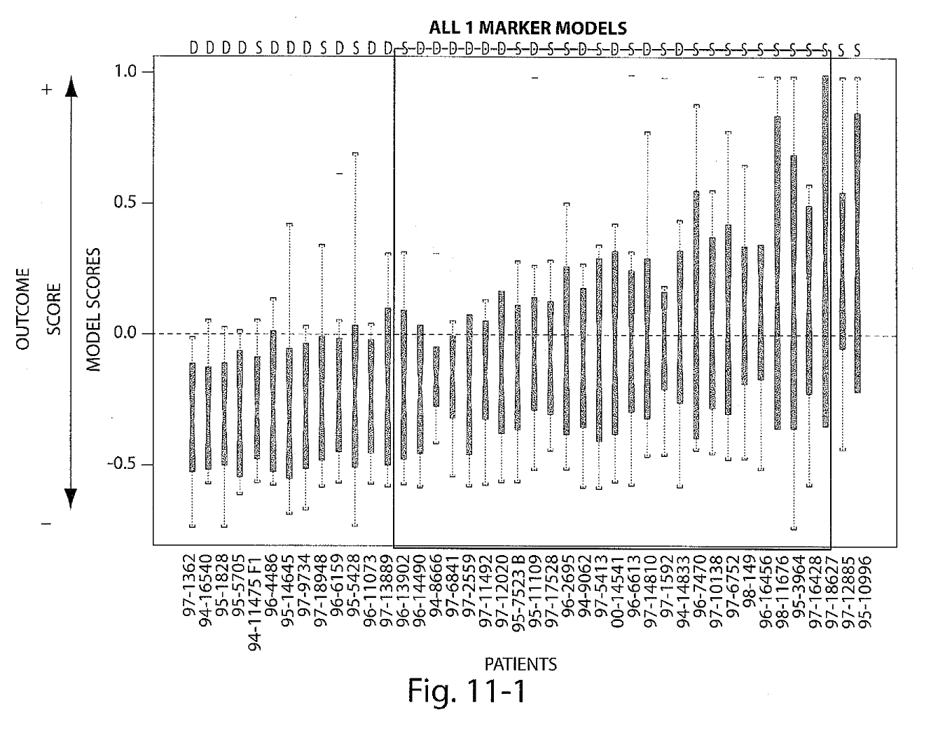

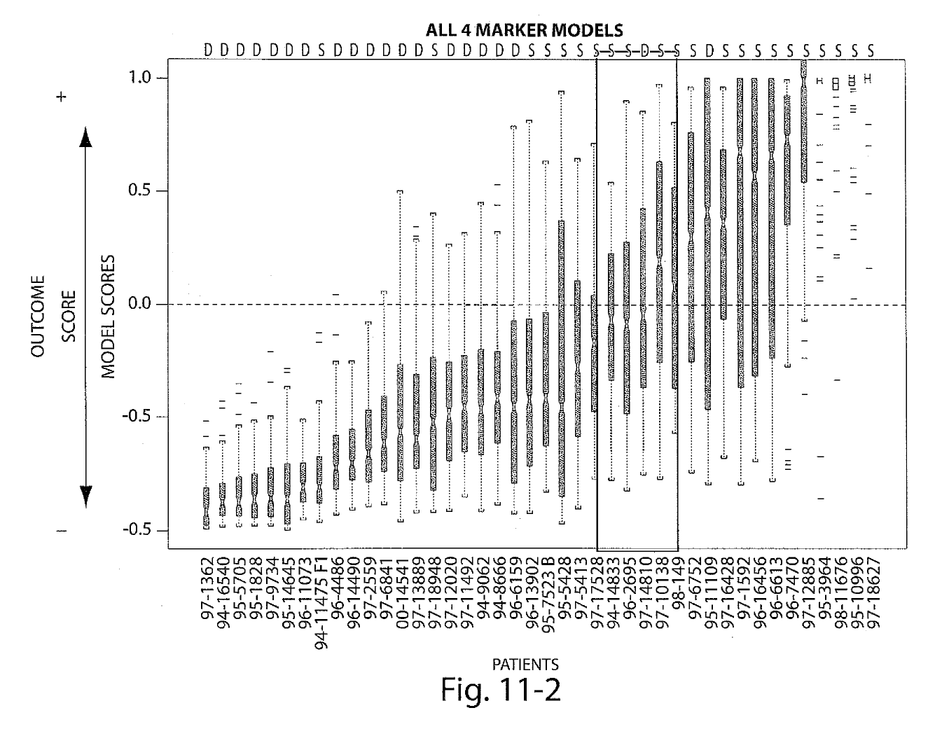

To demonstrate that multiple DNA repair biomarkers add a significant benefit in discriminating patient subgroups for Head and Neck cancer clinical decisions, the probability analysis was completed for all 1-, 2-, 3-, and 4-marker models in the HNCMARKERS set. The statistical parameters of p-value from Kaplan-Meier Overall survival curves, Relative Risk, Positive Predictive Value, and Average Error Rate (AER) were examined and plotted (FIG. 9). There is a distinctive overall improvement in statistical parameters by the increase from 1 to 4-HNCMARKER models. The median values show a decreased p-value, increased Relative Risk, increased Positive Predictive Value, and decreased AER within the HNCMARKER group (FIG. 9). By comparison, the combined analysis of all HNCMARKERS evaluated as individual biomarkers is less able to discriminate patient subsets for Overall Survival than a similar analysis on 4-HNCMARKER models. To illustrate this point, the diagram compares all 1 marker models in the HNCMARKER set with all 4 marker models in the HNCMARKER set (FIG. 11). The box denotes the patients on the X-axis for which there is mis-assignment into HIGH or LOW survival groups. Therefore, probability analysis indicates HNCMARKER combinations are able to reduce the confusion in the fraction of patients that are not correctly assigned by one result of the test (FIG. 10)

An additional demonstration of the importance of the multi-marker models is shown by considering one of the HNCMARKERS as a root marker for all subsequent models. The statistical values of p-value, Positive Predictive Value, and AER were computed for a 1-marker model with each of the FANCD2, XPF, ATM, BRCA1 or RAD51 biomarkers separately. Next, the same statistical tests were generated with all the 2-, 3- or 4-marker models containing the same starting biomarker, and the median value for all the related models was calculated, The same exercise was conducted for every root marker: FANCD2, XPF, ATM, BRCA1 or RAD51. In each of the five root marker cases, the 2-, 3- and 4-marker models show a trend to increased performance with addition of markers that is significantly improved over the related 1-marker models (FIG. 10). As for the calculations with all HNCMARKERS, increased performance features are associated with co-evaluation of markers in 2-, 3-, and 4-marker models.

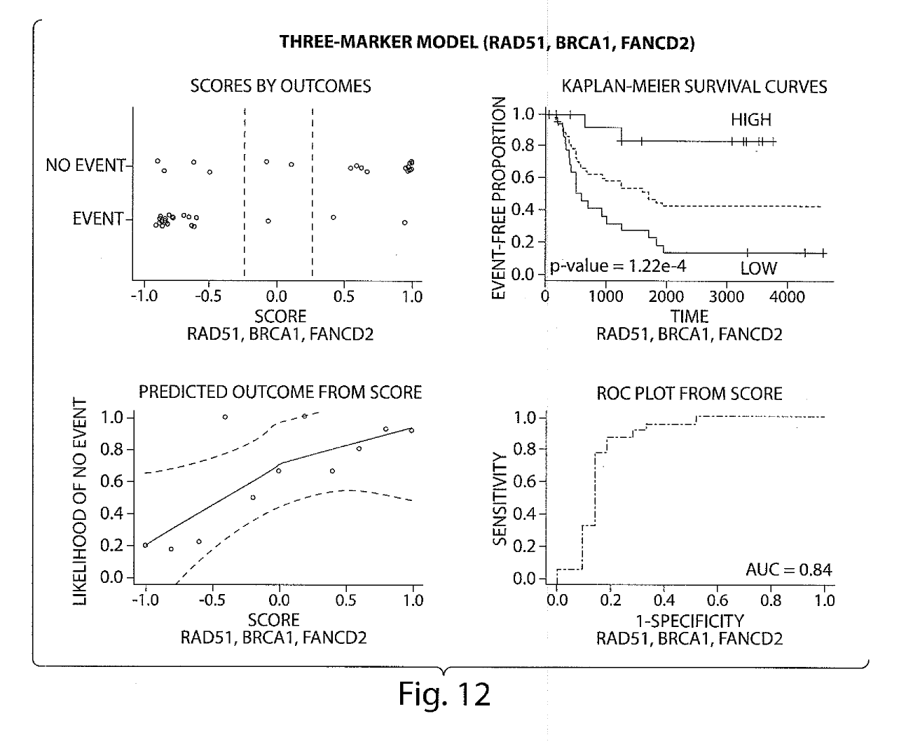

An example is shown with the RAD51, BRCA1, and FANCD2 markers from the HNCMARKER set (FIG. 12). For this three marker model, there was an increased significance for each of the statistical parameters assessed, including the Scores by Outcomes, Kaplan-Meier survival curve (p=1.22e-4), Predicted Outcome from Score, and ROC plot (AUC=0.84) indicative of better discrimination and fewer errors in a three HNCMARKER test over any of the single HNCMARKER tests (FIG. 12). In addition, the three marker models with HNCMARKERS illustrate the utility of identifying patient subgroups, as both the Good Survival (HIGH) and Poor Survival (LOW) subgroups are distinguished from the All Patients trend. General examples of DNA repair biomarker combinations are shown in the Table 8.

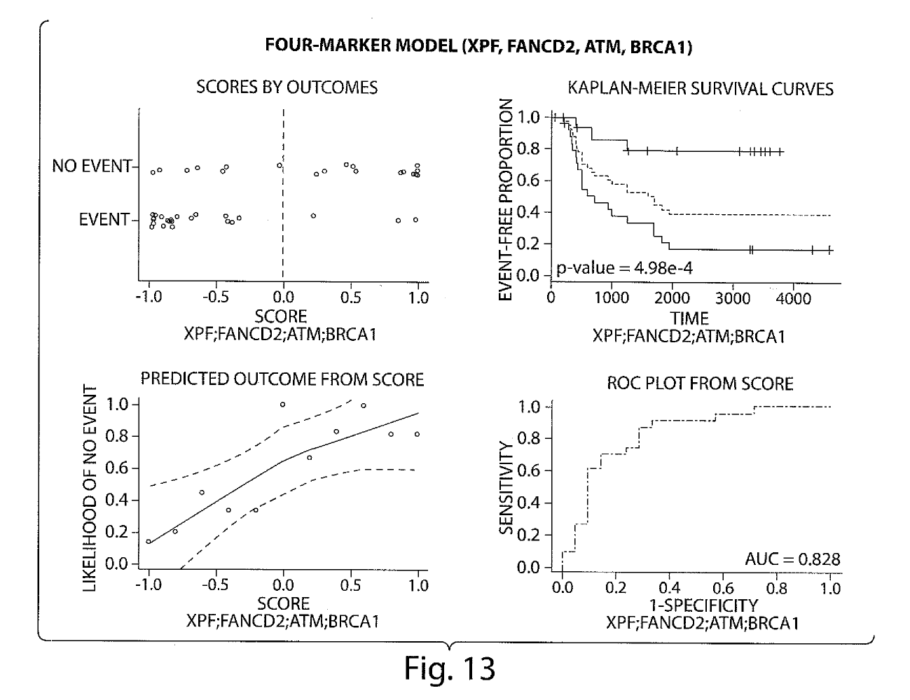

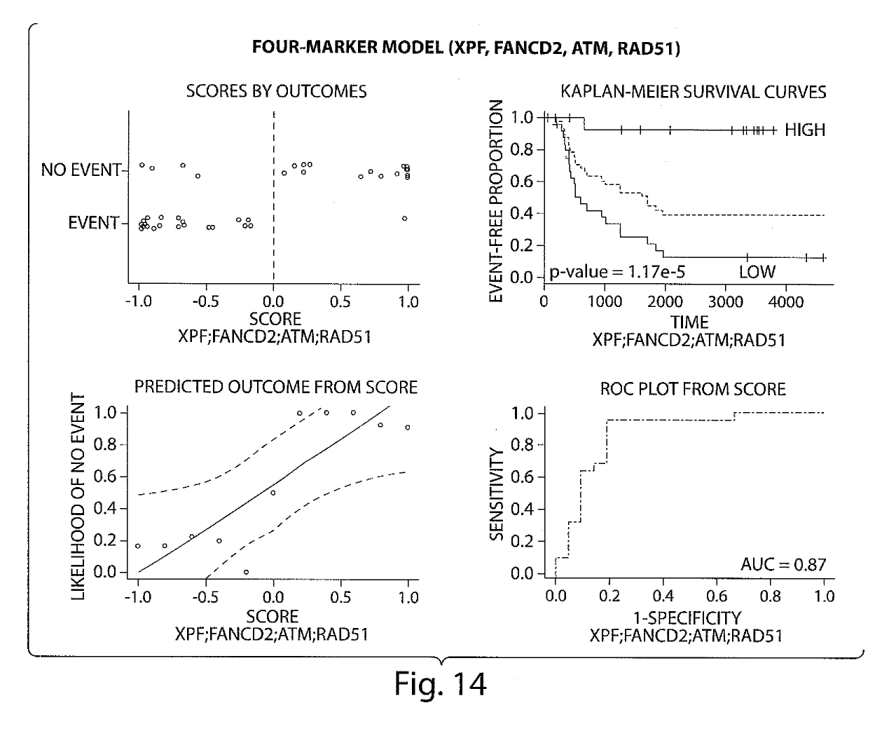

A multiple HNCMARKER Probability Analysis was also developed in four marker models from the HNCMARKER (Table 9). For the XPF, FANCD2, ATM, BRCA1 four marker model, there was an increased significance for the Scores by Outcomes, Kaplan-Meier survival curve (p=4.98e-4), Predicted Outcome from Score, and ROC plot (AUC=0.828) indicative of better discrimination and fewer errors in a four HNCMARKER test over any of the HNC single marker tests (FIG. 13). In an alternative XPF, FANCD2, RAD51, BRCA1 four marker model, there was an increased significance for the Scores by Outcomes, Kaplan-Meier survival curve (p=1.17e-5), Predicted Outcome from Score, and ROC plot (AUC=0.87) indicative of even better discrimination and fewer errors in a four HNCMARKER test over any of the HNC single marker tests (FIG. 14), which consistent with four marker partition analysis (Table 5). Therefore, the four HNCMARKER test gives better discrimination and fewer errors than a single HNCMARKER. In addition, the four marker models with HNCMARKERS illustrate the utility of identifying patient subgroups, as both the Good Survival (HIGH) and Poor Survival (LOW) subgroups are distinguished from the All Patients trend. In conclusion, multi-marker algorithms show that four-marker models are more specific, sensitive and statistically significant to distinguish survival groups in concurrent chemoradiotherapy. Tests from patient biopsies are relevant to delineating both good survival and poor survival patient subsets.

Several statistical tests were utilized to prove superiority over single biomarker values in a probability analysis. AUC values for the four individual markers were calculated for FANCD2 (0.73), XPF (0.69), and BRCA1 (0.75), RAD51 (0.68), ATM (0.65). By comparison, alternative four biomarker panels compared favorably with a significantly higher AUC value of 0.87 for the four DNA repair marker model (ATM; FANCD2; RAD51; XPF); AUC value of 0.86 for the four DNA repair marker model (BRCA1; FANCD2; RAD51; XPF); AUC value of 0.85 for the four DNA repair marker model (ATM; BRCA1; FANCD2; RAD51) and AUC value of 0.83 for the four DNA repair marker model (ATM; BRCA1; FANCD2; XPF) (Table 9). Likewise, Positive predictive power and negative predictive power calculations were evaluated. Individual markers showed Positive predictive power (0.59-0.69) and Negative predictive power (0.52-0.86). Instead, it was superior that the four marker algorithm of ATM; FANCD2; RAD51; XPF exhibited a Positive predictive power (0.84) and Negative predictive power (0.94), BRCA1; FANCD2; RAD51; XPF exhibited a Positive predictive power (0.79) and Negative predictive power (0.84), ATM; BRCA1; FANCD2; RAD51, exhibited a Positive predictive power (0.77) and Negative predictive power (0.88) and ATM; BRCA1; FANCD2; XPF exhibited a Positive predictive power (0.74) and Negative predictive power (0.82) (Table 9). As for other statistical metrics, the determinations of positive and negative predictive power proved that four marker models were more significant and reliable than testing with individual markers.

### Example 5

### HNCMARKERS are Discriminated with Additional Clinical Endpoints in Head and Neck Cancer Patient Benefit to Concurrent Chemoradiotherapy

Several clinical endpoints are evaluable with HNCMARKERS. Amongst these are Time to Progression, Disease-Free Survival, and Disease-Specific Survival and additional endpoints that are also significant in Head and Neck cancers. To illustrate that HNCMARKER algorithms are informative for additional clinical parameters, the following examples are described.

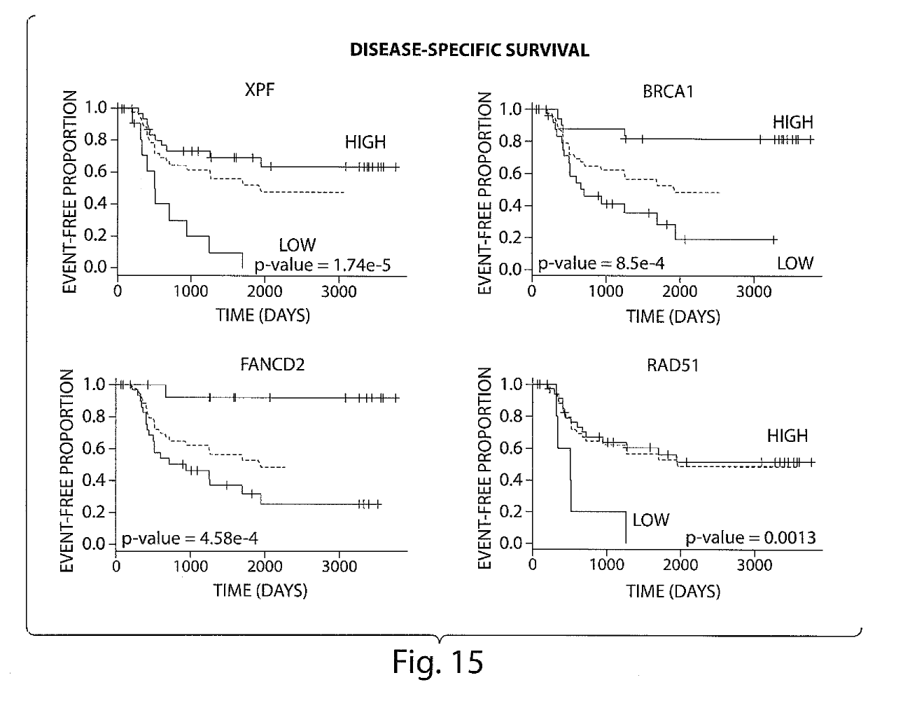

The Head and Neck cancer patients treated with concurrent chemotherapy were assessed for Disease-specific Survival using the Kaplan-Meier Disease-specific survival projections to inform the results of the algorithms. Partition analysis of single HNCMARKERS [XPF, BRCA1, FANCD2, and RAD51] were calculated, and they showed statistically significant delineation of the HIGH and LOW survival groups respectively: XPF (p-value=1.74e-5), BRCA1 (p-value=8.5e-4), FANCD2 (p-value=4.58e-4), RAD51 (p-value=0.0013) (FIG. 15). This data demonstrates that additional parameters such as Disease-Specific Survival are also evaluable with HNCMARKERS models for benefit of treatment with chemoradiotherapy.

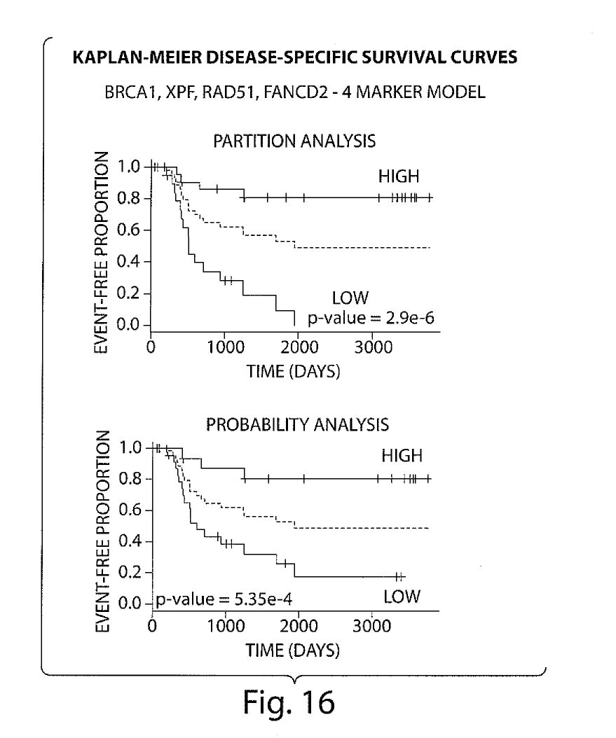

A second demonstration of utility is exhibited for HNCMARKERS and a Disease-Specific Survival evaluation when multimarker models are calculated. With the example of a 4-HNCMARKER model [BRCA1, XPF, RAD51, FANCD2], it is shown that the Head and Neck cancer patients treated with chemoradiotherapy are distinguished into High and Low survival groups (FIG. 16). The figure illustrates the benefit of the test for discriminating the survival groups by either the Partition or Probability analysis BRCA1, XPF, RAD51, FANCD2 4-marker model with high log 10 P-value significance for Partition (p=2.9e-06) and Probability (p=5.35e-4). Therefore, the HNCMARKER models are important and relevant to other clinical endpoints.

### Example 6

### HNCMARKERS that Discriminate Benefit of Induction Chemotherapy in Head and Neck Cancer

Univariate Analysis with Single HNCMARKERS

Eleven HNCMARKERS were analyzed for their ability to predict the responder and non-responder groups from induction chemotherapy (Table 10). The DNA repair biomarkers in the example include the following: XPF, pMK2, FANCD2, PARP1, ERCC1, MLH1, pH2AX, PAR, Ki67. In this example, two forms pMK2 are scored: cytoplasmic and nuclear. Similarly, two scores are evaluable for FANCD2, Nuclear foci (NF) and positive Nuclei (N). HNC patients are separated by partition analysis using fixed threshold strategy in evaluation of their response to induction chemotherapy.

Univariate Cox proportional hazards models were constructed for each of the markers to examine their potential predictive powers. ROC plots were constructed and the AUC computed for each of the markers. The AUC's were checked for significance using a permutations test. AUC values are shown from single biomarker ROC determinations. AUC: Area Under Curve, is a measure of how well separated two classes of data are under a testing scheme; ROC, a receiver operating characteristic, or simply ROC curve, is a graphical plot of the sensitivity vs. (1-specificity) for a binary classifier system as its discrimination threshold is varied. The ROC can also be represented equivalently by plotting the fraction of true positives (TPR=true positive rate) vs. the fraction of false positives (FPR=false positive rate). One of the markers was significant at the 95% confidence level ability for discriminating responders from non-responders (AUC=0.78, p=0.018) and one marker, pMK2, was significant at the 90% confidence level (AUC=0.71, p=0.08).

Quadratic discriminant models were constructed for each of the eleven biomarkers in the head and neck cancer study with equal costs for misclassification and priors assumed. To correct for the unequal group sizes the average error rate of classification for each marker was calculated and verified using Lachenbruch's holdout method. XPF had the lowest misclassification rate (observed 20%; crossvalidated 20%) of the eleven markers and pMK2 had the second lowest misclassification rate (observed 25%; crossvalidated 39%).

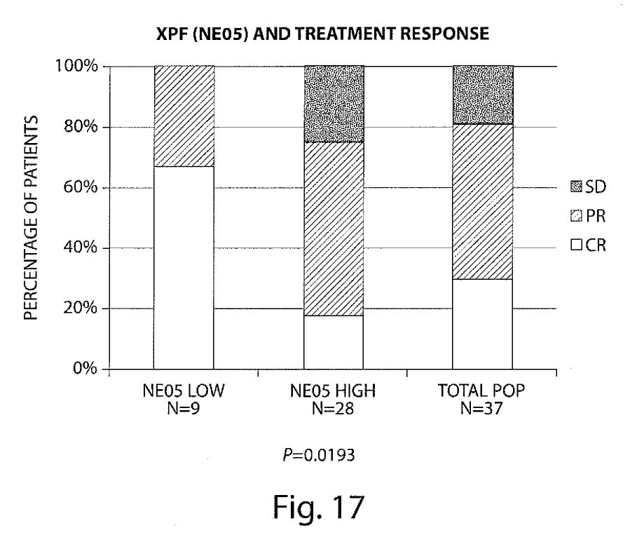

XPF is statistically significant by this test (FIG. 17). For confirmation, it was determined at the 80% confidence level after Bonferroni correction for multiple comparisons as well, suggesting that XPF is an important marker in predicting response to treatment in head and neck cancers. A complete response (CR) to induction chemotherapy is predictive of improved survival (Spaulding M B et al 1994, Jaulerry C et al 1992, Tejedor M et al 1992, Maipang T et al 1995).

### Role of Multiple Biomarkers in HNCMARKER Panels for Informing Response to Induction Chemotherapy in Head and Neck Cancers

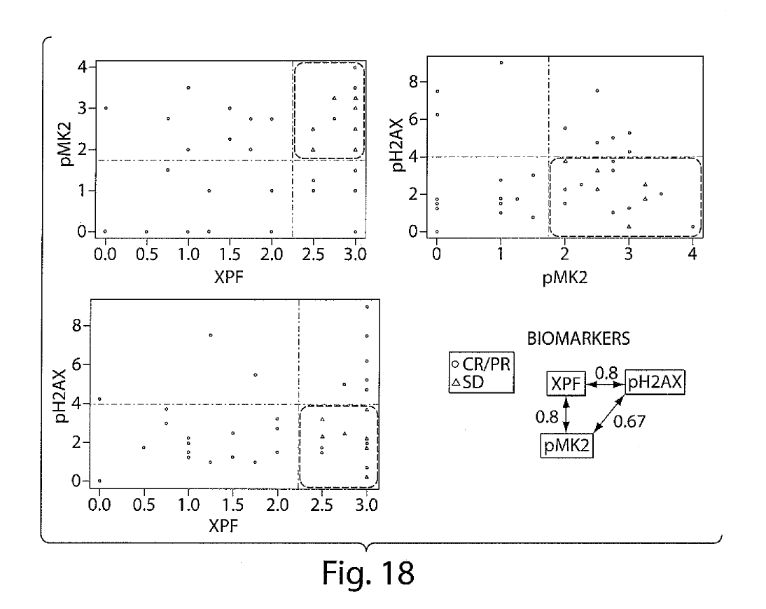

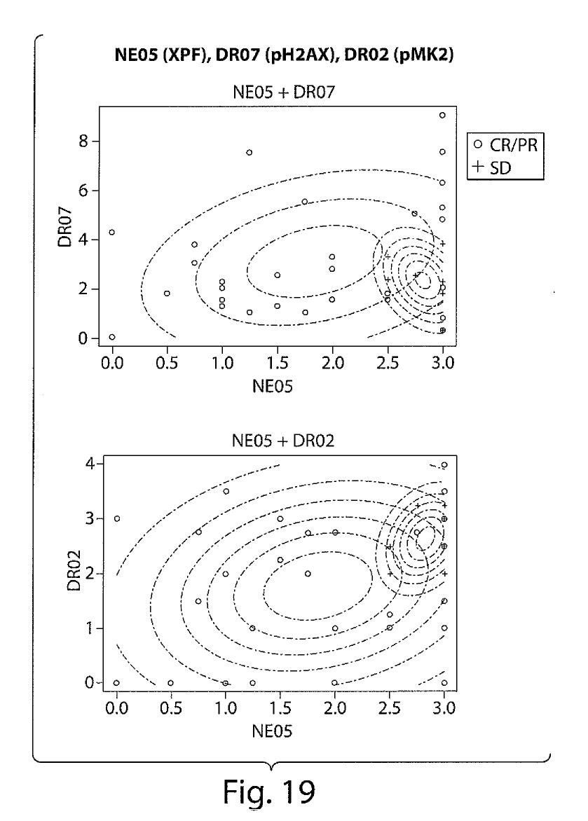

The DNA repair pathways may operate in cell survival and chemotherapy responses in a concerted way. Therefore, DNA repair protein changes may be more effectively determined by combining the effects of markers, rather than by individual analysis. In order to develop a statistically-driven hypothesis for these associations, the combination of two markers were analyzed in stepwise binary marker models using distributive partitioning. Models consisting of combinations of markers were constructed to investigate possible complimentary interactions between markers and pathways for discriminating responders from non-responders from induction chemotherapy in head and neck cancer. The outputting of marker comparisons showed good outcome from XPF, pH2AX, pMK2 based two-marker analysis (FIGS. 18, 19). Approximately 30% of the two marker models demonstrated higher AUC's than the best single marker model with the best performance attained by XPF and pH2AX (AUC=0.91). XPF appeared in 60% and pMK2 appeared in 33% of the top additive two marker models reinforcing their potential role in predicting response to treatment. XPF appeared in 38% of the top ratio models further implicating XPF is important in establishing a patient baseline response. Other markers did not perform consistently in similar pairwise tests, were not observed to belong to another group, and did not contribute to greater discrimination of the patient responder groups. All two marker models were computed for the HNCMARKERS; Statistical evaluation included p value, Apparent Error Rate, Relative Risk, Odds Ratio, Positive predictive power, and Negative predictive power.

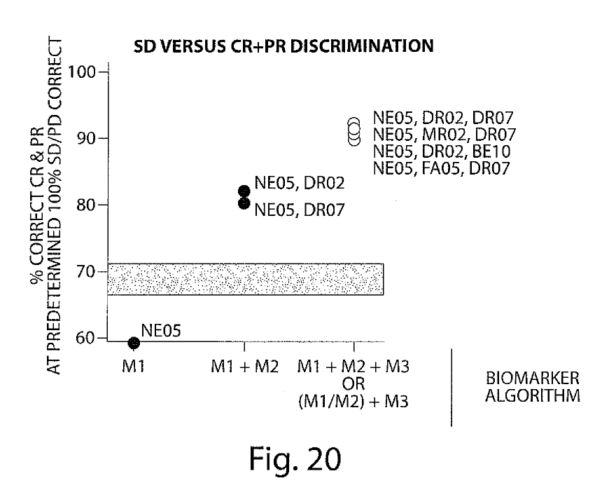

We extended the multi-marker models to include combinations of three markers, two possible ways to combine the markers were applied: (m₁+m₂+m₃) and (m₁/m₂+m₃). All three marker models were computed for the HNCMARKERS as predictors of induction chemotherapy benefit in Head and Neck cancer. Combinations of biomarkers yielded multiple biomarker models where a benefit for discriminating a patient response to induction chemotherapy in head and neck cancer could be determined (FIG. 20). The two different model structures yielded similar results when ROC plots were constructed with AUC's greater than 0.97 for top candidates in both model structures. The purely additive structure's top discriminant models yielded better performance for classifying the stable disease patients (71% correct vs 57% correct) but similar performance in classifying therapy responders correctly (87% correct vs 86% correct). XPF appeared in one third of the top 1% of the three marker models and pMK2 appeared in 14% of the top models. pH2AX was also identified as a common top marker occurring in 30% of the top models and present in the best performing three marker model for both structures. Multi-marker model analysis is more sensitive and effective to predict the response of head and neck cancer patients to the induction chemotherapy.

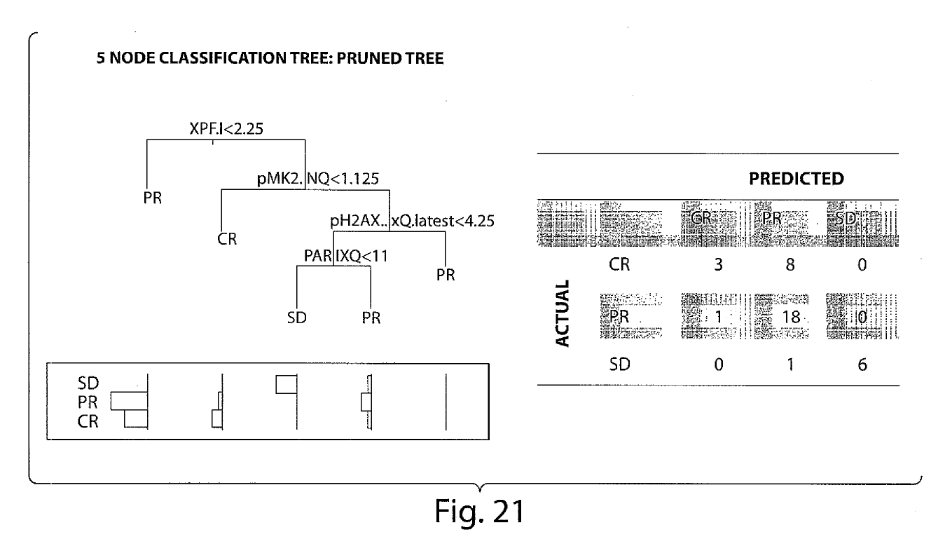

As an alternative discriminator of HNCMARKERS relevant to the induction chemotherapy response was also evaluated by use of classification subgrouping, FIG. 21 shows the multimarker projection in a pruned classification tree, and that HNCMARKERS are valid by pruned tree discriminators.

TABLE 1
HNCMARKERS in Head and Neck Cancer

| | | | BIOMARKER USED IN |
|---|---|---|---|
| BIOMARKER | CLASS | PATHWAY | THE TREATMENT |
| XPF | DNA repair | NER | 1, 2 |
| FANCD2 | DNA repair | FA/HR | 1, 2 |
| pMAPKAP | DNA damage | DDR | 1, 2 |
| Kinase 2 | signaling | | |
| (pMK2) | | | |
| pH2AX | DNA repair | DDR/NHEJ | 1, 2 |
| BRCA1 | DNA repair | FA/HR | 1, 2 |
| PAR | DNA repair | BER | 1, 2 |
| PARP1 | DNA repair | BER | 1 |
| MLH1 | DNA repair | MMR | 1 |
| ATM | DNA repair | DDR | 2 |
| ERCC1 | DNA repair | NER | 1, 2 |
| RAD51 | DNA repair | FA/HR | 2 |
| pHSP27 | Heat shock | DDR | 1 |
| | protein | | |
| p53 | Tumor | DDR | 2 |
| | supressor | | |
| POL H | DNA repair | TLS | 2 |
| MUS81 | DNA repair | NER | 1, 2 |
| Ki67 | Proliferation | | 1 |
| p16 | HPV marker | | 2 |
| HPV | HPV marker | | 2 |

1, Induction chemotherapy
2, Concurrent chemoradiotherapy (ChemoRT)

TABLE 2
One HNCMARKER Partition Analysis (Concurrent ChemoRT
Treatment and Overall Survival in Head and Neck Cancer)

| Markers | pval | AUC | Sens | Spec | PosPow | NegPow | AER | RelRisk |
|---|---|---|---|---|---|---|---|---|
| XPF | 1.72E−04 | 0.69 | 0.29 | 0.90 | 0.78 | 0.53 | 0.42 | 1.65 |
| FANCD2 | 1.96E−04 | 0.73 | 0.88 | 0.67 | 0.75 | 0.82 | 0.22 | 4.25 |
| RAD51 | 2.65E−03 | 0.71 | 0.23 | 0.95 | 0.83 | 0.53 | 0.43 | 1.76 |
| BRCA1 | 3.48E−03 | 0.74 | 0.87 | 0.62 | 0.71 | 0.81 | 0.25 | 3.81 |
| ATM | 2.03E−02 | 0.65 | 0.30 | 0.90 | 0.78 | 0.53 | 0.42 | 1.65 |
| PAR | 7.46E−02 | 0.52 | 1.00 | 0.30 | 0.61 | 1.00 | 0.33 | NE |
| p53 | 8.44E−02 | 0.55 | 0.91 | 0.30 | 0.60 | 0.75 | 0.37 | 2.40 |
| ERCC1 | 1.64E−01 | 0.56 | 0.22 | 0.95 | 0.83 | 0.51 | 0.44 | 1.71 |
| pH2AX | 1.93E−01 | 0.56 | 0.83 | 0.30 | 0.59 | 0.60 | 0.41 | 1.47 |
| pMK2 | 2.93E−01 | 0.51 | 0.83 | 0.27 | 0.54 | 0.60 | 0.44 | 1.36 |

TABLE 3
Two HNCMARKER Partition Analysis (Concurrent ChemoRT
Treatment and Overall Survival in Head and Neck Cancer)

| Markers | pval | AUC | Sens | Spec | PPV | NPV | AER | RelRisk |
|---|---|---|---|---|---|---|---|---|
| FANCD2, p53 | 7.12E−06 | | 0.91 | 0.75 | 0.81 | 0.88 | 0.16 | 6.87 |
| BRCA1, FANCD2 | 2.88E−05 | | 0.96 | 0.67 | 0.76 | 0.93 | 0.18 | 11.38 |
| FANCD2, XPF | 3.97E−05 | | 0.79 | 0.81 | 0.83 | 0.77 | 0.20 | 3.63 |
| FANCD2, RAD51 | 5.13E−05 | | 0.91 | 0.80 | 0.83 | 0.89 | 0.14 | 7.50 |
| FANCD2, pH2AX | 7.89E−05 | | 0.88 | 0.70 | 0.78 | 0.82 | 0.20 | 4.41 |
| RAD51, XPF | 8.25E−05 | | 0.41 | 0.90 | 0.82 | 0.58 | 0.36 | 1.95 |
| XPF, p53 | 8.97E−05 | | 0.43 | 0.90 | 0.83 | 0.58 | 0.35 | 1.99 |
| BRCA1, XPF | 1.02E−04 | | 0.74 | 0.86 | 0.85 | 0.75 | 0.20 | 3.40 |
| FANCD2, PAR | 1.09E−04 | | 0.86 | 0.70 | 0.76 | 0.82 | 0.21 | 4.31 |
| FANCD2, pMK2 | 1.19E−04 | | 0.91 | 0.67 | 0.75 | 0.88 | 0.20 | 6.00 |
| ERCC1, FANCD2 | 1.28E−04 | | 0.87 | 0.70 | 0.77 | 0.82 | 0.21 | 4.36 |
| ATM, RAD51 | 1.45E−04 | | 0.59 | 0.95 | 0.93 | 0.68 | 0.24 | 2.89 |
| ATM, PAR | 1.61E−04 | | 0.29 | 0.95 | 0.86 | 0.56 | 0.39 | 1.94 |
| BRCA1, RAD51 | 1.68E−04 | | 0.86 | 0.80 | 0.83 | 0.84 | 0.17 | 5.23 |
| XPF, pH2AX | 1.78E−04 | | 0.25 | 0.90 | 0.75 | 0.50 | 0.45 | 1.50 |
| PAR, XPF | 1.84E−04 | | 0.23 | 0.90 | 0.71 | 0.51 | 0.45 | 1.47 |
| XPF, pMK2 | 2.51E−04 | | 0.30 | 0.90 | 0.78 | 0.54 | 0.41 | 1.70 |
| ATM, FANCD2 | 2.53E−04 | | 0.91 | 0.65 | 0.75 | 0.87 | 0.21 | 5.63 |
| RAD51, p53 | 3.02E−04 | | 0.73 | 0.75 | 0.76 | 0.71 | 0.26 | 2.67 |
| BRCA1, p53 | 3.30E−04 | | 0.78 | 0.80 | 0.82 | 0.76 | 0.21 | 3.44 |
| ATM, XPF | 3.67E−04 | | 0.30 | 0.90 | 0.78 | 0.53 | 0.42 | 1.65 |
| ERCC1, XPF | 3.67E−04 | | 0.30 | 0.90 | 0.78 | 0.53 | 0.42 | 1.65 |
| RAD51, pH2AX | 4.50E−04 | | 0.32 | 0.95 | 0.88 | 0.56 | 0.38 | 1.98 |
| BRCA1, PAR | 6.98E−04 | | 0.81 | 0.75 | 0.77 | 0.79 | 0.22 | 3.67 |
| ATM, ERCC1 | 7.05E−04 | | 0.30 | 0.95 | 0.88 | 0.54 | 0.40 | 1.91 |
| BRCA1, pH2AX | 1.97E−03 | | 0.87 | 0.65 | 0.74 | 0.81 | 0.23 | 3.95 |
| PAR, p53 | 2.07E−03 | | 0.95 | 0.45 | 0.65 | 0.90 | 0.29 | 6.45 |
| ATM, pMK2 | 2.53E−03 | | 0.57 | 0.85 | 0.81 | 0.63 | 0.30 | 2.19 |
| BRCA1, pMK2 | 2.58E−03 | | 0.74 | 0.76 | 0.77 | 0.73 | 0.25 | 2.83 |
| p53, pH2AX | 2.62E−03 | | 0.65 | 0.65 | 0.68 | 0.62 | 0.35 | 1.79 |
| ERCC1, RAD51 | 2.65E−03 | | 0.23 | 0.95 | 0.83 | 0.53 | 0.43 | 1.76 |
| RAD51, pMK2 | 2.65E−03 | | 0.23 | 0.95 | 0.83 | 0.53 | 0.43 | 1.76 |
| BRCA1, ERCC1 | 3.27E−03 | | 0.65 | 0.85 | 0.83 | 0.68 | 0.26 | 2.60 |
| ATM, BRCA1 | 5.82E−03 | | 0.61 | 0.80 | 0.78 | 0.64 | 0.30 | 2.16 |
| ATM, p53 | 5.82E−03 | | 0.61 | 0.80 | 0.78 | 0.64 | 0.30 | 2.16 |
| ATM, pH2AX | 5.82E−03 | | 0.61 | 0.80 | 0.78 | 0.64 | 0.30 | 2.16 |
| PAR, RAD51 | 6.18E−03 | | 0.95 | 0.60 | 0.70 | 0.92 | 0.22 | 9.15 |
| pMK2, pH2AX | 1.01E−02 | | 0.30 | 0.95 | 0.88 | 0.54 | 0.40 | 1.91 |
| PAR, pH2AX | 1.31E−02 | | 1.00 | 0.30 | 0.61 | 1.00 | 0.33 | NE |
| p53, pMK2 | 2.56E−02 | | 0.22 | 0.95 | 0.83 | 0.51 | 0.44 | 1.71 |
| ERCC1, p53 | 3.36E−02 | | 0.61 | 0.65 | 0.67 | 0.59 | 0.37 | 1.63 |
| PAR, pMK2 | 4.84E−02 | | 0.86 | 0.40 | 0.60 | 0.73 | 0.37 | 2.20 |
| ERCC1, PAR | 8.00E−02 | | 1.00 | 0.30 | 0.60 | 1.00 | 0.34 | NE |
| ERCC1, pH2AX | 8.35E−02 | | 0.35 | 0.90 | 0.80 | 0.55 | 0.40 | 1.76 |
| ERCC1, pMK2 | 1.64E−01 | | 0.22 | 0.95 | 0.83 | 0.51 | 0.44 | 1.71 |

TABLE 4
Three HNCMARKER Partition Analysis (ChemoRT Treatment and
Overall Survival in Head and Neck Cancer)

| Markers | pval | AUC | Sens | Spec | PosPow | NegPow | AER | RelRisk |
|---|---|---|---|---|---|---|---|---|
| RAD51, XPF, pH2AX | 6.18E−08 | | 0.23 | 0.95 | 0.83 | 0.53 | 0.43 | 1.76 |
| FANCD2, RAD51, p53 | 2.98E−07 | | 0.82 | 0.90 | 0.90 | 0.82 | 0.14 | 4.95 |
| FANCD2, p53, pH2AX | 6.68E−07 | | 0.87 | 0.80 | 0.83 | 0.84 | 0.16 | 5.28 |
| BRCA1, FANCD2, p53 | 7.83E−07 | | 0.87 | 0.80 | 0.83 | 0.84 | 0.16 | 5.28 |
| FANCD2, PAR, p53 | 8.10E−07 | | 0.86 | 0.80 | 0.82 | 0.84 | 0.17 | 5.18 |
| BRCA1, RAD51, p53 | 8.68E−07 | | 0.82 | 0.90 | 0.90 | 0.82 | 0.14 | 4.95 |
| ERCC1, FANCD2, p53 | 1.80E−06 | | 0.83 | 0.80 | 0.83 | 0.80 | 0.19 | 4.13 |
| BRCA1, p53, pMK2 | 3.55E−06 | | 0.26 | 1.00 | 1.00 | 0.54 | 0.40 | 2.18 |
| BRCA1, FANCD2, XPF | 3.82E−06 | | 0.83 | 0.86 | 0.86 | 0.82 | 0.16 | 4.75 |
| FANCD2, XPF, p53 | 3.82E−06 | | 0.83 | 0.85 | 0.86 | 0.81 | 0.16 | 4.53 |
| FANCD2, XPF, pH2AX | 3.82E−06 | | 0.79 | 0.85 | 0.86 | 0.77 | 0.18 | 3.80 |
| FANCD2, RAD51, pH2AX | 5.89E−06 | | 0.82 | 0.85 | 0.86 | 0.81 | 0.17 | 4.50 |
| ATM, FANCD2, p53 | 6.62E−06 | | 0.91 | 0.75 | 0.81 | 0.88 | 0.16 | 6.87 |
| BRCA1, FANCD2, RAD51 | 6.90E−06 | | 0.82 | 0.85 | 0.86 | 0.81 | 0.17 | 4.50 |
| RAD51, p53, pMK2 | 7.25E−06 | | 0.59 | 0.85 | 0.81 | 0.65 | 0.29 | 2.35 |
| FANCD2, p53, pMK2 | 8.34E−06 | | 0.96 | 0.70 | 0.79 | 0.93 | 0.16 | 11.79 |
| BRCA1, RAD51, XPF | 1.12E−05 | | 0.27 | 0.95 | 0.86 | 0.54 | 0.40 | 1.88 |
| FANCD2, PAR, pH2AX | 1.12E−05 | | 0.82 | 0.75 | 0.78 | 0.79 | 0.21 | 3.72 |
| BRCA1, RAD51, pH2AX | 1.16E−05 | | 0.86 | 0.85 | 0.86 | 0.85 | 0.14 | 5.76 |
| FANCD2, RAD51, XPF | 1.29E−05 | | 0.64 | 0.90 | 0.88 | 0.69 | 0.24 | 2.84 |
| RAD51, XPF, p53 | 1.29E−05 | | 0.64 | 0.90 | 0.88 | 0.69 | 0.24 | 2.84 |
| BRCA1, FANCD2, PAR | 1.30E−05 | | 0.86 | 0.75 | 0.78 | 0.83 | 0.20 | 4.70 |
| BRCA1, FANCD2, pH2AX | 1.61E−05 | | 0.91 | 0.70 | 0.78 | 0.88 | 0.19 | 6.22 |
| BRCA1, PAR, RAD51 | 1.91E−05 | | 0.85 | 0.85 | 0.85 | 0.85 | 0.15 | 5.67 |
| ERCC1, FANCD2, pH2AX | 2.17E−05 | | 0.83 | 0.75 | 0.79 | 0.79 | 0.21 | 3.76 |
| BRCA1, XPF, p53 | 2.39E−05 | | 0.78 | 0.85 | 0.86 | 0.77 | 0.19 | 3.77 |
| BRCA1, XPF, pH2AX | 2.39E−05 | | 0.78 | 0.85 | 0.86 | 0.77 | 0.19 | 3.77 |
| BRCA1, ERCC1, FANCD2 | 2.47E−05 | | 0.83 | 0.75 | 0.79 | 0.79 | 0.21 | 3.76 |
| BRCA1, p53, pH2AX | 3.39E−05 | | 0.61 | 0.95 | 0.93 | 0.68 | 0.23 | 2.90 |
| PAR, RAD51, p53 | 3.60E−05 | | 0.40 | 1.00 | 1.00 | 0.63 | 0.30 | 2.67 |
| BRCA1, PAR, XPF | 3.72E−05 | | 0.76 | 0.85 | 0.84 | 0.77 | 0.20 | 3.71 |
| ATM, FANCD2, XPF | 3.97E−05 | | 0.83 | 0.80 | 0.83 | 0.80 | 0.19 | 4.13 |
| ERCC1, FANCD2, XPF | 3.97E−05 | | 0.83 | 0.80 | 0.83 | 0.80 | 0.19 | 4.13 |
| FANCD2, XPF, pMK2 | 3.97E−05 | | 0.83 | 0.81 | 0.83 | 0.81 | 0.18 | 4.34 |
| ATM, XPF, p53 | 4.54E−05 | | 0.43 | 0.90 | 0.83 | 0.58 | 0.35 | 1.99 |
| ERCC1, XPF, p53 | 4.54E−05 | | 0.43 | 0.90 | 0.83 | 0.58 | 0.35 | 1.99 |
| XPF, p53, pH2AX | 4.54E−05 | | 0.43 | 0.90 | 0.83 | 0.58 | 0.35 | 1.99 |
| XPF, p53, pMK2 | 4.54E−05 | | 0.43 | 0.90 | 0.83 | 0.58 | 0.35 | 1.99 |
| ATM, FANCD2, RAD51 | 5.05E−05 | | 0.95 | 0.75 | 0.81 | 0.94 | 0.14 | 12.92 |
| ERCC1, FANCD2, RAD51 | 5.05E−05 | | 0.95 | 0.75 | 0.81 | 0.94 | 0.14 | 12.92 |
| FANCD2, RAD51, pMK2 | 5.05E−05 | | 0.95 | 0.75 | 0.81 | 0.94 | 0.14 | 12.92 |
| ATM, FANCD2, pH2AX | 5.12E−05 | | 0.96 | 0.65 | 0.76 | 0.93 | 0.19 | 10.62 |
| FANCD2, pMK2, pH2AX | 5.12E−05 | | 0.96 | 0.65 | 0.76 | 0.93 | 0.19 | 10.62 |
| FANCD2, PAR, XPF | 5.29E−05 | | 0.36 | 0.90 | 0.80 | 0.56 | 0.38 | 1.83 |
| PAR, XPF, p53 | 5.29E−05 | | 0.38 | 0.90 | 0.80 | 0.58 | 0.37 | 1.91 |
| RAD51, p53, pH2AX | 6.41E−05 | | 0.50 | 0.85 | 0.79 | 0.61 | 0.33 | 2.00 |
| BRCA1, PAR, p53 | 6.41E−05 | | 0.57 | 0.95 | 0.92 | 0.68 | 0.24 | 2.87 |
| ERCC1, RAD51, XPF | 6.63E−05 | | 0.32 | 0.90 | 0.78 | 0.55 | 0.40 | 1.71 |
| ATM, FANCD2, PAR | 6.93E−05 | | 0.95 | 0.65 | 0.74 | 0.93 | 0.20 | 10.37 |
| ERCC1, FANCD2, PAR | 6.93E−05 | | 0.95 | 0.65 | 0.74 | 0.93 | 0.20 | 10.37 |
| FANCD2, PAR, pMK2 | 6.93E−05 | | 0.95 | 0.65 | 0.74 | 0.93 | 0.20 | 10.37 |
| FANCD2, PAR, RAD51 | 6.95E−05 | | 0.95 | 0.75 | 0.79 | 0.94 | 0.15 | 12.67 |
| ATM, BRCA1, FANCD2 | 7.30E−05 | | 0.87 | 0.70 | 0.77 | 0.82 | 0.21 | 4.36 |
| BRCA1, FANCD2, pMK2 | 7.30E−05 | | 0.87 | 0.71 | 0.77 | 0.83 | 0.20 | 4.62 |
| ATM, BRCA1, RAD51 | 8.09E−05 | | 0.86 | 0.80 | 0.83 | 0.84 | 0.17 | 5.23 |
| BRCA1, ERCC1, RAD51 | 8.09E−05 | | 0.86 | 0.80 | 0.83 | 0.84 | 0.17 | 5.23 |
| BRCA1, RAD51, pMK2 | 8.09E−05 | | 0.86 | 0.80 | 0.83 | 0.84 | 0.17 | 5.23 |
| PAR, RAD51, XPF | 8.23E−05 | | 0.65 | 0.90 | 0.87 | 0.72 | 0.22 | 3.10 |
| BRCA1, XPF, pMK2 | 8.35E−05 | | 0.65 | 0.90 | 0.88 | 0.70 | 0.23 | 2.98 |
| RAD51, XPF, pMK2 | 1.10E−04 | | 0.36 | 0.90 | 0.80 | 0.56 | 0.38 | 1.83 |
| BRCA1, ERCC1, p53 | 1.15E−04 | | 0.83 | 0.75 | 0.79 | 0.79 | 0.21 | 3.76 |
| ATM, ERCC1, FANCD2 | 1.25E−04 | | 0.87 | 0.70 | 0.77 | 0.82 | 0.21 | 4.36 |
| BRCA1, ERCC1, XPF | 1.37E−04 | | 0.61 | 0.90 | 0.88 | 0.67 | 0.26 | 2.63 |
| ERCC1, FANCD2, pMK2 | 1.38E−04 | | 0.91 | 0.65 | 0.75 | 0.87 | 0.21 | 5.63 |
| ERCC1, RAD51, p53 | 1.42E−04 | | 0.59 | 0.80 | 0.76 | 0.64 | 0.31 | 2.12 |
| ATM, ERCC1, XPF | 1.72E−04 | | 0.30 | 0.90 | 0.78 | 0.53 | 0.42 | 1.65 |
| ATM, XPF, pMK2 | 1.72E−04 | | 0.30 | 0.90 | 0.78 | 0.53 | 0.42 | 1.65 |
| ERCC1, XPF, pMK2 | 1.72E−04 | | 0.30 | 0.90 | 0.78 | 0.53 | 0.42 | 1.65 |
| ATM, XPF, pH2AX | 1.74E−04 | | 0.26 | 1.00 | 1.00 | 0.54 | 0.40 | 2.18 |
| ERCC1, XPF, pH2AX | 1.74E−04 | | 0.26 | 1.00 | 1.00 | 0.54 | 0.40 | 2.18 |
| XPF, pMK2, pH2AX | 1.74E−04 | | 0.26 | 1.00 | 1.00 | 0.54 | 0.40 | 2.18 |
| ATM, BRCA1, XPF | 1.98E−04 | | 0.78 | 0.80 | 0.82 | 0.76 | 0.21 | 3.44 |
| ATM, FANCD2, pMK2 | 2.40E−04 | | 0.96 | 0.60 | 0.73 | 0.92 | 0.21 | 9.53 |
| ATM, BRCA1, p53 | 2.43E−04 | | 0.87 | 0.70 | 0.77 | 0.82 | 0.21 | 4.36 |
| ATM, RAD51, XPF | 2.47E−04 | | 0.64 | 0.85 | 0.82 | 0.68 | 0.26 | 2.57 |
| ATM, RAD51, p53 | 2.68E−04 | | 0.55 | 0.95 | 0.92 | 0.66 | 0.26 | 2.68 |
| PAR, XPF, pH2AX | 3.27E−04 | | 0.18 | 0.90 | 0.67 | 0.50 | 0.48 | 1.33 |
| ERCC1, PAR, XPF | 3.81E−04 | | 0.29 | 1.00 | 1.00 | 0.57 | 0.37 | 2.33 |
| PAR, XPF, pMK2 | 3.81E−04 | | 0.29 | 1.00 | 1.00 | 0.57 | 0.37 | 2.33 |
| BRCA1, PAR, pH2AX | 3.98E−04 | | 0.86 | 0.70 | 0.75 | 0.82 | 0.22 | 4.25 |
| ATM, RAD51, pH2AX | 4.50E−04 | | 0.32 | 0.95 | 0.88 | 0.56 | 0.38 | 1.98 |
| ERCC1, RAD51, pH2AX | 4.50E−04 | | 0.32 | 0.95 | 0.88 | 0.56 | 0.38 | 1.98 |
| RAD51, pMK2, pH2AX | 4.50E−04 | | 0.32 | 0.95 | 0.88 | 0.56 | 0.38 | 1.98 |
| BRCA1, pMK2, pH2AX | 5.13E−04 | | 0.26 | 1.00 | 1.00 | 0.54 | 0.40 | 2.18 |
| BRCA1, ERCC1, pH2AX | 5.94E−04 | | 0.83 | 0.70 | 0.76 | 0.78 | 0.23 | 3.42 |
| ATM, ERCC1, RAD51 | 7.05E−04 | | 0.32 | 1.00 | 1.00 | 0.57 | 0.36 | 2.33 |
| ATM, PAR, RAD51 | 7.05E−04 | | 0.35 | 1.00 | 1.00 | 0.61 | 0.32 | 2.54 |
| ATM, RAD51, pMK2 | 7.05E−04 | | 0.32 | 1.00 | 1.00 | 0.57 | 0.36 | 2.33 |
| p53, pMK2, pH2AX | 8.90E−04 | | 0.26 | 1.00 | 1.00 | 0.54 | 0.40 | 2.18 |
| BRCA1, ERCC1, PAR | 9.43E−04 | | 0.81 | 0.75 | 0.77 | 0.79 | 0.22 | 3.67 |
| ATM, BRCA1, pH2AX | 1.02E−03 | | 0.87 | 0.65 | 0.74 | 0.81 | 0.23 | 3.95 |
| PAR, RAD51, pH2AX | 1.52E−03 | | 0.45 | 0.95 | 0.90 | 0.63 | 0.30 | 2.45 |
| ATM, PAR, XPF | 1.61E−03 | | 0.38 | 1.00 | 1.00 | 0.61 | 0.32 | 2.54 |
| PAR, p53, pH2AX | 1.62E−03 | | 0.52 | 0.70 | 0.65 | 0.58 | 0.39 | 1.55 |
| ERCC1, PAR, RAD51 | 1.67E−03 | | 0.30 | 1.00 | 1.00 | 0.59 | 0.35 | 2.43 |
| ATM, BRCA1, ERCC1 | 1.79E−03 | | 0.35 | 1.00 | 1.00 | 0.57 | 0.35 | 2.33 |
| ATM, BRCA1, PAR | 1.79E−03 | | 0.38 | 1.00 | 1.00 | 0.61 | 0.32 | 2.54 |
| BRCA1, ERCC1, pMK2 | 2.03E−03 | | 0.65 | 0.75 | 0.75 | 0.65 | 0.30 | 2.16 |
| ERCC1, RAD51, pMK2 | 2.65E−03 | | 0.23 | 0.95 | 0.83 | 0.53 | 0.43 | 1.76 |
| ATM, ERCC1, p53 | 3.23E−03 | | 0.43 | 0.95 | 0.91 | 0.59 | 0.33 | 2.24 |
| BRCA1, PAR, pMK2 | 3.32E−03 | | 0.67 | 0.80 | 0.78 | 0.70 | 0.27 | 2.56 |
| ATM, BRCA1, pMK2 | 3.48E−03 | | 0.87 | 0.60 | 0.71 | 0.80 | 0.26 | 3.57 |
| ERCC1, p53, pH2AX | 3.77E−03 | | 0.48 | 0.70 | 0.65 | 0.54 | 0.42 | 1.40 |
| ATM, ERCC1, pMK2 | 4.85E−03 | | 0.30 | 0.95 | 0.88 | 0.54 | 0.40 | 1.91 |
| ATM, PAR, p53 | 5.82E−03 | | 0.67 | 0.80 | 0.78 | 0.70 | 0.27 | 2.56 |
| ERCC1, p53, pMK2 | 5.82E−03 | | 0.78 | 0.55 | 0.67 | 0.69 | 0.33 | 2.13 |
| ATM, p53, pMK2 | 5.91E−03 | | 0.57 | 0.90 | 0.87 | 0.64 | 0.28 | 2.43 |
| PAR, p53, pMK2 | 6.07E−03 | | 0.81 | 0.70 | 0.74 | 0.78 | 0.24 | 3.33 |
| ATM, PAR, pMK2 | 6.67E−03 | | 0.33 | 0.95 | 0.88 | 0.58 | 0.37 | 2.06 |
| ATM, ERCC1, PAR | 7.07E−03 | | 0.29 | 1.00 | 1.00 | 0.57 | 0.37 | 2.33 |
| ATM, ERCC1, pH2AX | 7.07E−03 | | 0.26 | 1.00 | 1.00 | 0.54 | 0.40 | 2.18 |
| PAR, RAD51, pMK2 | 7.50E−03 | | 0.80 | 0.75 | 0.76 | 0.79 | 0.22 | 3.62 |
| ERCC1, PAR, p53 | 8.09E−03 | | 0.57 | 0.80 | 0.75 | 0.64 | 0.32 | 2.08 |
| ATM, p53, pH2AX | 1.09E−02 | | 0.74 | 0.65 | 0.71 | 0.68 | 0.30 | 2.24 |
| ERCC1, PAR, pMK2 | 1.99E−02 | | 0.24 | 0.95 | 0.83 | 0.54 | 0.41 | 1.82 |
| ATM, PAR, pH2AX | 2.60E−02 | | 0.24 | 0.95 | 0.83 | 0.54 | 0.41 | 1.82 |
| ATM, pMK2, pH2AX | 3.16E−02 | | 0.61 | 0.75 | 0.74 | 0.63 | 0.33 | 1.96 |
| ERCC1, PAR, pH2AX | 3.69E−02 | | 0.95 | 0.45 | 0.65 | 0.90 | 0.29 | 6.45 |
| PAR, pMK2, pH2AX | 4.27E−02 | | 0.33 | 0.95 | 0.88 | 0.58 | 0.37 | 2.06 |
| ERCC1, pMK2, pH2AX | 4.98E−02 | | 0.26 | 0.95 | 0.86 | 0.53 | 0.42 | 1.82 |

TABLE 5
Four HNCMARKER Partition Analysis (ChemoRT Treatment and
Overall Survival in Head and Neck Cancer)

| Markers | pval | AUC | Sens | Spec | PosPow | NegPow | AER | RelRisk |
|---|---|---|---|---|---|---|---|---|
| FANCD2, RAD51, p53, pH2AX | 1.55E−08 | | 0.86 | 0.90 | 0.90 | 0.86 | 0.12 | 6.33 |
| FANCD2, PAR, p53, pH2AX | 2.64E−08 | | 0.86 | 0.85 | 0.86 | 0.85 | 0.15 | 5.71 |
| BRCA1, FANCD2, RAD51, p53 | 8.46E−08 | | 0.91 | 0.85 | 0.87 | 0.89 | 0.12 | 8.26 |
| BRCA1, FANCD2, p53, pH2AX | 1.21E−07 | | 0.91 | 0.80 | 0.84 | 0.89 | 0.14 | 7.56 |
| BRCA1, FANCD2, PAR, p53 | 1.31E−07 | | 0.90 | 0.80 | 0.83 | 0.89 | 0.15 | 7.43 |
| FANCD2, XPF, p53, pH2AX | 1.94E−07 | | 0.83 | 0.85 | 0.86 | 0.81 | 0.16 | 4.53 |
| BRCA1, FANCD2, XPF, p53 | 2.76E−07 | | 0.83 | 0.85 | 0.86 | 0.81 | 0.16 | 4.53 |
| FANCD2, p53, pMK2, pH2AX | 4.37E−07 | | 0.70 | 0.90 | 0.89 | 0.72 | 0.21 | 3.17 |
| ATM, FANCD2, RAD51, p53 | 4.86E−07 | | 0.86 | 0.85 | 0.86 | 0.85 | 0.14 | 5.76 |
| ERCC1, FANCD2, RAD51, p53 | 4.86E−07 | | 0.86 | 0.85 | 0.86 | 0.85 | 0.14 | 5.76 |
| FANCD2, RAD51, XPF, p53 | 4.86E−07 | | 0.86 | 0.85 | 0.86 | 0.85 | 0.14 | 5.76 |
| FANCD2, RAD51, p53, pMK2 | 4.86E−07 | | 0.86 | 0.85 | 0.86 | 0.85 | 0.14 | 5.76 |
| BRCA1, RAD51, p53, pH2AX | 5.73E−07 | | 0.73 | 0.95 | 0.94 | 0.76 | 0.17 | 3.92 |
| ATM, FANCD2, p53, pH2AX | 5.93E−07 | | 0.91 | 0.80 | 0.84 | 0.89 | 0.14 | 7.56 |
| FANCD2, PAR, RAD51, p53 | 6.05E−07 | | 0.85 | 0.85 | 0.85 | 0.85 | 0.15 | 5.67 |
| ERCC1, FANCD2, p53, pH2AX | 6.68E−07 | | 0.87 | 0.80 | 0.83 | 0.84 | 0.16 | 5.28 |
| ATM, FANCD2, PAR, p53 | 7.64E−07 | | 0.90 | 0.80 | 0.83 | 0.89 | 0.15 | 7.43 |
| ERCC1, FANCD2, PAR, p53 | 8.10E−07 | | 0.86 | 0.80 | 0.82 | 0.84 | 0.17 | 5.18 |
| FANCD2, PAR, XPF, p53 | 8.10E−07 | | 0.86 | 0.80 | 0.82 | 0.84 | 0.17 | 5.18 |
| FANCD2, PAR, p53, pMK2 | 8.10E−07 | | 0.86 | 0.80 | 0.82 | 0.84 | 0.17 | 5.18 |
| BRCA1, PAR, RAD51, p53 | 1.07E−06 | | 0.70 | 0.95 | 0.93 | 0.76 | 0.18 | 3.89 |
| BRCA1, FANCD2, RAD51, pH2AX | 1.45E−06 | | 0.91 | 0.80 | 0.83 | 0.89 | 0.14 | 7.50 |
| ATM, BRCA1, FANCD2, p53 | 1.80E−06 | | 0.91 | 0.75 | 0.81 | 0.88 | 0.16 | 6.87 |
| BRCA1, ERCC1, FANCD2, p53 | 1.80E−06 | | 0.91 | 0.75 | 0.81 | 0.88 | 0.16 | 6.87 |
| BRCA1, FANCD2, p53, pMK2 | 1.80E−06 | | 0.91 | 0.75 | 0.81 | 0.88 | 0.16 | 6.87 |
| BRCA1, FANCD2, PAR, pH2AX | 2.20E−06 | | 0.90 | 0.75 | 0.79 | 0.88 | 0.17 | 6.73 |
| ATM, FANCD2, p53, pMK2 | 2.73E−06 | | 0.30 | 1.00 | 1.00 | 0.56 | 0.37 | 2.25 |
| ERCC1, FANCD2, p53, pMK2 | 2.73E−06 | | 0.30 | 1.00 | 1.00 | 0.56 | 0.37 | 2.25 |
| ATM, FANCD2, XPF, p53 | 3.46E−06 | | 0.87 | 0.80 | 0.83 | 0.84 | 0.16 | 5.28 |
| BRCA1, p53, pMK2, pH2AX | 3.55E−06 | | 0.26 | 1.00 | 1.00 | 0.54 | 0.40 | 2.18 |
| ERCC1, FANCD2, XPF, p53 | 3.82E−06 | | 0.83 | 0.80 | 0.83 | 0.80 | 0.19 | 4.13 |
| FANCD2, XPF, p53, pMK2 | 3.82E−06 | | 0.83 | 0.80 | 0.83 | 0.80 | 0.19 | 4.13 |
| ATM, FANCD2, RAD51, pH2AX | 6.24E−06 | | 0.91 | 0.80 | 0.83 | 0.89 | 0.14 | 7.50 |
| ATM, ERCC1, FANCD2, p53 | 6.62E−06 | | 0.91 | 0.75 | 0.81 | 0.88 | 0.16 | 6.87 |
| ERCC1, FANCD2, RAD51, pH2AX | 7.00E−06 | | 0.86 | 0.80 | 0.83 | 0.84 | 0.17 | 5.23 |
| FANCD2, RAD51, XPF, pH2AX | 7.00E−06 | | 0.86 | 0.80 | 0.83 | 0.84 | 0.17 | 5.23 |
| FANCD2, RAD51, pMK2, pH2AX | 7.00E−06 | | 0.86 | 0.80 | 0.83 | 0.84 | 0.17 | 5.23 |
| ATM, FANCD2, PAR, pH2AX | 9.07E−06 | | 0.90 | 0.75 | 0.79 | 0.88 | 0.17 | 6.73 |
| FANCD2, PAR, RAD51, pH2AX | 9.58E−06 | | 0.85 | 0.80 | 0.81 | 0.84 | 0.18 | 5.13 |
| BRCA1, FANCD2, XPF, pH2AX | 9.59E−06 | | 0.87 | 0.75 | 0.80 | 0.83 | 0.19 | 4.80 |
| ERCC1, FANCD2, PAR, pH2AX | 1.12E−05 | | 0.95 | 0.70 | 0.77 | 0.93 | 0.17 | 11.54 |
| FANCD2, PAR, pMK2, pH2AX | 1.12E−05 | | 0.95 | 0.70 | 0.77 | 0.93 | 0.17 | 11.54 |
| FANCD2, PAR, XPF, pH2AX | 1.12E−05 | | 0.82 | 0.75 | 0.78 | 0.79 | 0.21 | 3.72 |
| ATM, BRCA1, RAD51, p53 | 1.20E−05 | | 0.82 | 0.85 | 0.86 | 0.81 | 0.17 | 4.50 |
| BRCA1, ERCC1, RAD51, p53 | 1.20E−05 | | 0.82 | 0.85 | 0.86 | 0.81 | 0.17 | 4.50 |
| BRCA1, RAD51, p53, pMK2 | 1.20E−05 | | 0.82 | 0.85 | 0.86 | 0.81 | 0.17 | 4.50 |
| ATM, FANCD2, RAD51, XPF | 1.29E−05 | | 0.64 | 0.90 | 0.88 | 0.69 | 0.24 | 2.84 |
| ATM, RAD51, XPF, p53 | 1.29E−05 | | 0.64 | 0.90 | 0.88 | 0.69 | 0.24 | 2.84 |
| ERCC1, FANCD2, RAD51, XPF | 1.29E−05 | | 0.64 | 0.90 | 0.88 | 0.69 | 0.24 | 2.84 |
| ERCC1, RAD51, XPF, p53 | 1.29E−05 | | 0.64 | 0.90 | 0.88 | 0.69 | 0.24 | 2.84 |
| FANCD2, RAD51, XPF, pMK2 | 1.29E−05 | | 0.64 | 0.90 | 0.88 | 0.69 | 0.24 | 2.84 |
| RAD51, XPF, p53, pH2AX | 1.29E−05 | | 0.64 | 0.90 | 0.88 | 0.69 | 0.24 | 2.84 |
| RAD51, XPF, p53, pMK2 | 1.29E−05 | | 0.64 | 0.90 | 0.88 | 0.69 | 0.24 | 2.84 |
| ATM, FANCD2, XPF, pH2AX | 1.30E−05 | | 0.78 | 0.85 | 0.86 | 0.77 | 0.19 | 3.77 |
| ERCC1, FANCD2, XPF, pH2AX | 1.30E−05 | | 0.78 | 0.85 | 0.86 | 0.77 | 0.19 | 3.77 |
| FANCD2, XPF, pMK2, pH2AX | 1.30E−05 | | 0.78 | 0.85 | 0.86 | 0.77 | 0.19 | 3.77 |
| ATM, BRCA1, FANCD2, RAD51 | 1.42E−05 | | 0.91 | 0.75 | 0.80 | 0.88 | 0.17 | 6.80 |
| BRCA1, ERCC1, FANCD2, RAD51 | 1.42E−05 | | 0.91 | 0.75 | 0.80 | 0.88 | 0.17 | 6.80 |
| BRCA1, FANCD2, RAD51, XPF | 1.42E−05 | | 0.91 | 0.75 | 0.80 | 0.88 | 0.17 | 6.80 |
| BRCA1, FANCD2, RAD51, pMK2 | 1.42E−05 | | 0.91 | 0.75 | 0.80 | 0.88 | 0.17 | 6.80 |
| BRCA1, XPF, p53, pH2AX | 1.45E−05 | | 0.70 | 0.90 | 0.89 | 0.72 | 0.21 | 3.17 |
| BRCA1, RAD51, XPF, p53 | 1.45E−05 | | 0.82 | 0.85 | 0.86 | 0.81 | 0.17 | 4.50 |
| PAR, RAD51, XPF, p53 | 1.47E−05 | | 0.40 | 1.00 | 1.00 | 0.63 | 0.30 | 2.67 |
| ATM, BRCA1, FANCD2, pH2AX | 1.61E−05 | | 0.91 | 0.70 | 0.78 | 0.88 | 0.19 | 6.22 |
| BRCA1, ERCC1, FANCD2, pH2AX | 1.61E−05 | | 0.91 | 0.70 | 0.78 | 0.88 | 0.19 | 6.22 |
| BRCA1, FANCD2, pMK2, pH2AX | 1.61E−05 | | 0.91 | 0.70 | 0.78 | 0.88 | 0.19 | 6.22 |
| BRCA1, FANCD2, PAR, RAD51 | 1.87E−05 | | 0.90 | 0.75 | 0.78 | 0.88 | 0.18 | 6.65 |
| FANCD2, PAR, RAD51, XPF | 1.98E−05 | | 0.60 | 0.90 | 0.86 | 0.69 | 0.25 | 2.79 |
| ATM, BRCA1, FANCD2, PAR | 2.10E−05 | | 0.90 | 0.70 | 0.76 | 0.88 | 0.20 | 6.08 |
| BRCA1, ERCC1, FANCD2, PAR | 2.10E−05 | | 0.90 | 0.70 | 0.76 | 0.88 | 0.20 | 6.08 |
| BRCA1, FANCD2, PAR, XPF | 2.10E−05 | | 0.90 | 0.70 | 0.76 | 0.88 | 0.20 | 6.08 |
| BRCA1, FANCD2, PAR, pMK2 | 2.10E−05 | | 0.90 | 0.70 | 0.76 | 0.88 | 0.20 | 6.08 |
| BRCA1, PAR, XPF, p53 | 2.20E−05 | | 0.67 | 0.90 | 0.88 | 0.72 | 0.22 | 3.12 |
| BRCA1, PAR, p53, pH2AX | 2.97E−05 | | 0.76 | 0.85 | 0.84 | 0.77 | 0.20 | 3.71 |
| ATM, BRCA1, p53, pH2AX | 3.39E−05 | | 0.61 | 0.95 | 0.93 | 0.68 | 0.23 | 2.90 |
| BRCA1, ERCC1, p53, pH2AX | 3.39E−05 | | 0.61 | 0.95 | 0.93 | 0.68 | 0.23 | 2.90 |
| ATM, PAR, RAD51, p53 | 3.60E−05 | | 0.40 | 1.00 | 1.00 | 0.63 | 0.30 | 2.67 |
| ERCC1, PAR, RAD51, p53 | 3.60E−05 | | 0.40 | 1.00 | 1.00 | 0.63 | 0.30 | 2.67 |
| PAR, RAD51, p53, pH2AX | 3.60E−05 | | 0.40 | 1.00 | 1.00 | 0.63 | 0.30 | 2.67 |
| PAR, RAD51, p53, pMK2 | 3.60E−05 | | 0.40 | 1.00 | 1.00 | 0.63 | 0.30 | 2.67 |
| ATM, BRCA1, XPF, p53 | 4.54E−05 | | 0.43 | 0.95 | 0.91 | 0.59 | 0.33 | 2.24 |
| ATM, ERCC1, FANCD2, XPF | 4.54E−05 | | 0.43 | 0.90 | 0.83 | 0.58 | 0.35 | 1.99 |
| ATM, ERCC1, XPF, p53 | 4.54E−05 | | 0.43 | 0.90 | 0.83 | 0.58 | 0.35 | 1.99 |
| ATM, FANCD2, XPF, pMK2 | 4.54E−05 | | 0.43 | 0.90 | 0.83 | 0.58 | 0.35 | 1.99 |
| ATM, XPF, p53, pH2AX | 4.54E−05 | | 0.43 | 0.90 | 0.83 | 0.58 | 0.35 | 1.99 |
| ATM, XPF, p53, pMK2 | 4.54E−05 | | 0.43 | 0.90 | 0.83 | 0.58 | 0.35 | 1.99 |
| BRCA1, ERCC1, XPF, p53 | 4.54E−05 | | 0.43 | 0.95 | 0.91 | 0.59 | 0.33 | 2.24 |
| BRCA1, XPF, p53, pMK2 | 4.54E−05 | | 0.43 | 0.95 | 0.91 | 0.59 | 0.33 | 2.24 |
| ERCC1, FANCD2, XPF, pMK2 | 4.54E−05 | | 0.43 | 0.90 | 0.83 | 0.58 | 0.35 | 1.99 |
| ERCC1, XPF, p53, pH2AX | 4.54E−05 | | 0.43 | 0.90 | 0.83 | 0.58 | 0.35 | 1.99 |
| ERCC1, XPF, p53, pMK2 | 4.54E−05 | | 0.43 | 0.90 | 0.83 | 0.58 | 0.35 | 1.99 |
| XPF, p53, pMK2, pH2AX | 4.54E−05 | | 0.43 | 0.90 | 0.83 | 0.58 | 0.35 | 1.99 |
| ATM, BRCA1, FANCD2, XPF | 5.00E−05 | | 0.83 | 0.75 | 0.79 | 0.79 | 0.21 | 3.76 |
| BRCA1, ERCC1, FANCD2, XPF | 5.00E−05 | | 0.83 | 0.75 | 0.79 | 0.79 | 0.21 | 3.76 |
| BRCA1, FANCD2, XPF, pMK2 | 5.00E−05 | | 0.83 | 0.76 | 0.79 | 0.80 | 0.20 | 3.96 |
| ATM, ERCC1, FANCD2, RAD51 | 5.05E−05 | | 0.95 | 0.70 | 0.78 | 0.93 | 0.17 | 11.67 |
| ATM, FANCD2, RAD51, pMK2 | 5.05E−05 | | 0.95 | 0.70 | 0.78 | 0.93 | 0.17 | 11.67 |
| ERCC1, FANCD2, RAD51, pMK2 | 5.05E−05 | | 0.95 | 0.70 | 0.78 | 0.93 | 0.17 | 11.67 |
| ATM, ERCC1, FANCD2, pH2AX | 5.12E−05 | | 0.96 | 0.65 | 0.76 | 0.93 | 0.19 | 10.62 |
| ATM, FANCD2, pMK2, pH2AX | 5.12E−05 | | 0.96 | 0.65 | 0.76 | 0.93 | 0.19 | 10.62 |
| ERCC1, FANCD2, pMK2, pH2AX | 5.12E−05 | | 0.96 | 0.65 | 0.76 | 0.93 | 0.19 | 10.62 |
| ATM, PAR, XPF, p53 | 5.29E−05 | | 0.38 | 0.90 | 0.80 | 0.58 | 0.37 | 1.91 |
| ERCC1, PAR, XPF, p53 | 5.29E−05 | | 0.38 | 0.90 | 0.80 | 0.58 | 0.37 | 1.91 |
| PAR, XPF, p53, pH2AX | 5.29E−05 | | 0.38 | 0.90 | 0.80 | 0.58 | 0.37 | 1.91 |
| PAR, XPF, p53, pMK2 | 5.29E−05 | | 0.38 | 0.90 | 0.80 | 0.58 | 0.37 | 1.91 |
| BRCA1, ERCC1, PAR, RAD51 | 6.14E−05 | | 0.50 | 1.00 | 1.00 | 0.67 | 0.25 | 3.00 |
| ATM, BRCA1, PAR, p53 | 6.41E−05 | | 0.57 | 0.95 | 0.92 | 0.68 | 0.24 | 2.87 |
| BRCA1, PAR, p53, pMK2 | 6.41E−05 | | 0.57 | 0.95 | 0.92 | 0.68 | 0.24 | 2.87 |
| ATM, ERCC1, FANCD2, PAR | 6.93E−05 | | 0.95 | 0.65 | 0.74 | 0.93 | 0.20 | 10.37 |
| ATM, FANCD2, PAR, XPF | 6.93E−05 | | 0.95 | 0.65 | 0.74 | 0.93 | 0.20 | 10.37 |
| ATM, FANCD2, PAR, pMK2 | 6.93E−05 | | 0.95 | 0.65 | 0.74 | 0.93 | 0.20 | 10.37 |
| ERCC1, FANCD2, PAR, XPF | 6.93E−05 | | 0.95 | 0.65 | 0.74 | 0.93 | 0.20 | 10.37 |
| ERCC1, FANCD2, PAR, pMK2 | 6.93E−05 | | 0.95 | 0.65 | 0.74 | 0.93 | 0.20 | 10.37 |
| FANCD2, PAR, XPF, pMK2 | 6.93E−05 | | 0.95 | 0.65 | 0.74 | 0.93 | 0.20 | 10.37 |
| ATM, FANCD2, PAR, RAD51 | 6.95E−05 | | 0.95 | 0.70 | 0.76 | 0.93 | 0.18 | 11.40 |
| ERCC1, FANCD2, PAR, RAD51 | 6.95E−05 | | 0.95 | 0.70 | 0.76 | 0.93 | 0.18 | 11.40 |
| FANCD2, PAR, RAD51, pMK2 | 6.95E−05 | | 0.95 | 0.70 | 0.76 | 0.93 | 0.18 | 11.40 |
| BRCA1, RAD51, XPF, pH2AX | 8.72E−05 | | 0.27 | 0.95 | 0.86 | 0.54 | 0.40 | 1.88 |
| ATM, BRCA1, RAD51, pH2AX | 9.64E−05 | | 0.82 | 0.80 | 0.82 | 0.80 | 0.19 | 4.09 |
| BRCA1, ERCC1, RAD51, pH2AX | 9.64E−05 | | 0.82 | 0.80 | 0.82 | 0.80 | 0.19 | 4.09 |
| BRCA1, RAD51, pMK2, pH2AX | 9.64E−05 | | 0.82 | 0.80 | 0.82 | 0.80 | 0.19 | 4.09 |
| ATM, BRCA1, ERCC1, FANCD2 | 9.96E−05 | | 0.91 | 0.65 | 0.75 | 0.87 | 0.21 | 5.63 |
| ATM, BRCA1, FANCD2, pMK2 | 9.96E−05 | | 0.91 | 0.65 | 0.75 | 0.87 | 0.21 | 5.63 |
| BRCA1, ERCC1, FANCD2, pMK2 | 9.96E−05 | | 0.91 | 0.65 | 0.75 | 0.87 | 0.21 | 5.63 |
| BRCA1, PAR, RAD51, pH2AX | 1.24E−04 | | 0.95 | 0.60 | 0.70 | 0.92 | 0.22 | 9.15 |
| ATM, RAD51, XPF, pH2AX | 1.27E−04 | | 0.27 | 0.95 | 0.86 | 0.54 | 0.40 | 1.88 |
| ERCC1, RAD51, XPF, pH2AX | 1.27E−04 | | 0.27 | 0.95 | 0.86 | 0.54 | 0.40 | 1.88 |
| RAD51, XPF, pMK2, pH2AX | 1.27E−04 | | 0.27 | 0.95 | 0.86 | 0.54 | 0.40 | 1.88 |
| ATM, BRCA1, PAR, RAD51 | 1.69E−04 | | 0.80 | 0.80 | 0.80 | 0.80 | 0.20 | 4.00 |
| BRCA1, PAR, RAD51, pMK2 | 1.69E−04 | | 0.80 | 0.80 | 0.80 | 0.80 | 0.20 | 4.00 |
| ATM, BRCA1, ERCC1, XPF | 1.72E−04 | | 0.30 | 0.95 | 0.88 | 0.54 | 0.40 | 1.91 |
| ATM, BRCA1, XPF, pMK2 | 1.72E−04 | | 0.30 | 0.95 | 0.88 | 0.54 | 0.40 | 1.91 |
| ATM, ERCC1, XPF, pH2AX | 1.72E−04 | | 0.30 | 0.90 | 0.78 | 0.53 | 0.42 | 1.65 |
| ATM, ERCC1, XPF, pMK2 | 1.72E−04 | | 0.30 | 0.90 | 0.78 | 0.53 | 0.42 | 1.65 |
| ATM, XPF, pMK2, pH2AX | 1.72E−04 | | 0.30 | 0.90 | 0.78 | 0.53 | 0.42 | 1.65 |
| BRCA1, ERCC1, XPF, pMK2 | 1.72E−04 | | 0.30 | 0.95 | 0.88 | 0.54 | 0.40 | 1.91 |
| ERCC1, XPF, pMK2, pH2AX | 1.72E−04 | | 0.30 | 0.90 | 0.78 | 0.53 | 0.42 | 1.65 |
| BRCA1, ERCC1, PAR, p53 | 1.72E−04 | | 0.86 | 0.65 | 0.72 | 0.81 | 0.24 | 3.84 |
| ATM, BRCA1, XPF, pH2AX | 1.78E−04 | | 0.70 | 0.85 | 0.84 | 0.71 | 0.23 | 2.89 |
| BRCA1, ERCC1, XPF, pH2AX | 1.78E−04 | | 0.70 | 0.85 | 0.84 | 0.71 | 0.23 | 2.89 |
| BRCA1, XPF, pMK2, pH2AX | 1.78E−04 | | 0.70 | 0.85 | 0.84 | 0.71 | 0.23 | 2.89 |
| ATM, RAD51, p53, pH2AX | 1.94E−04 | | 0.73 | 0.85 | 0.84 | 0.74 | 0.21 | 3.23 |
| BRCA1, PAR, RAD51, XPF | 2.02E−04 | | 0.80 | 0.80 | 0.80 | 0.80 | 0.20 | 4.00 |
| ATM, ERCC1, FANCD2, pMK2 | 2.40E−04 | | 0.96 | 0.60 | 0.73 | 0.92 | 0.21 | 9.53 |
| ATM, BRCA1, RAD51, XPF | 2.47E−04 | | 0.64 | 0.90 | 0.88 | 0.69 | 0.24 | 2.84 |
| ATM, ERCC1, RAD51, XPF | 2.47E−04 | | 0.64 | 0.85 | 0.82 | 0.68 | 0.26 | 2.57 |
| ATM, RAD51, XPF, pMK2 | 2.47E−04 | | 0.64 | 0.85 | 0.82 | 0.68 | 0.26 | 2.57 |
| BRCA1, ERCC1, RAD51, XPF | 2.47E−04 | | 0.64 | 0.90 | 0.88 | 0.69 | 0.24 | 2.84 |
| BRCA1, RAD51, XPF, pMK2 | 2.47E−04 | | 0.64 | 0.90 | 0.88 | 0.69 | 0.24 | 2.84 |
| ERCC1, RAD51, XPF, pMK2 | 2.47E−04 | | 0.64 | 0.85 | 0.82 | 0.68 | 0.26 | 2.57 |
| BRCA1, ERCC1, p53, pMK2 | 2.71E−04 | | 0.57 | 0.85 | 0.81 | 0.63 | 0.30 | 2.19 |
| ATM, BRCA1, PAR, XPF | 2.96E−04 | | 0.67 | 0.85 | 0.82 | 0.71 | 0.24 | 2.82 |
| BRCA1, ERCC1, PAR, XPF | 2.96E−04 | | 0.67 | 0.85 | 0.82 | 0.71 | 0.24 | 2.82 |
| BRCA1, PAR, XPF, pH2AX | 2.96E−04 | | 0.67 | 0.85 | 0.82 | 0.71 | 0.24 | 2.82 |
| BRCA1, PAR, XPF, pMK2 | 3.27E−04 | | 0.57 | 0.95 | 0.92 | 0.68 | 0.24 | 2.87 |
| ATM, PAR, XPF, pMK2 | 3.81E−04 | | 0.29 | 1.00 | 1.00 | 0.57 | 0.37 | 2.33 |
| ERCC1, PAR, XPF, pMK2 | 3.81E−04 | | 0.29 | 1.00 | 1.00 | 0.57 | 0.37 | 2.33 |
| PAR, XPF, pMK2, pH2AX | 3.81E−04 | | 0.29 | 1.00 | 1.00 | 0.57 | 0.37 | 2.33 |
| ATM, BRCA1, ERCC1, p53 | 4.38E−04 | | 0.61 | 0.90 | 0.88 | 0.67 | 0.26 | 2.63 |
| ATM, BRCA1, p53, pMK2 | 4.38E−04 | | 0.61 | 0.90 | 0.88 | 0.67 | 0.26 | 2.63 |
| ERCC1, RAD51, p53, pH2AX | 4.39E−04 | | 0.91 | 0.65 | 0.74 | 0.87 | 0.21 | 5.56 |
| RAD51, p53, pMK2, pH2AX | 4.39E−04 | | 0.91 | 0.65 | 0.74 | 0.87 | 0.21 | 5.56 |
| ATM, PAR, RAD51, XPF | 4.49E−04 | | 0.60 | 0.85 | 0.80 | 0.68 | 0.28 | 2.50 |
| ERCC1, PAR, RAD51, XPF | 4.49E−04 | | 0.60 | 0.85 | 0.80 | 0.68 | 0.28 | 2.50 |
| PAR, RAD51, XPF, pH2AX | 4.49E−04 | | 0.60 | 0.85 | 0.80 | 0.68 | 0.28 | 2.50 |
| PAR, RAD51, XPF, pMK2 | 4.49E−04 | | 0.60 | 0.85 | 0.80 | 0.68 | 0.28 | 2.50 |
| ATM, ERCC1, RAD51, pH2AX | 4.50E−04 | | 0.32 | 0.95 | 0.88 | 0.56 | 0.38 | 1.98 |
| ATM, RAD51, pMK2, pH2AX | 4.50E−04 | | 0.32 | 0.95 | 0.88 | 0.56 | 0.38 | 1.98 |
| ERCC1, RAD51, pMK2, pH2AX | 4.50E−04 | | 0.32 | 0.95 | 0.88 | 0.56 | 0.38 | 1.98 |
| ATM, BRCA1, ERCC1, RAD51 | 5.24E−04 | | 0.82 | 0.75 | 0.78 | 0.79 | 0.21 | 3.72 |
| ATM, BRCA1, RAD51, pMK2 | 5.24E−04 | | 0.82 | 0.75 | 0.78 | 0.79 | 0.21 | 3.72 |
| BRCA1, ERCC1, RAD51, pMK2 | 5.24E−04 | | 0.82 | 0.75 | 0.78 | 0.79 | 0.21 | 3.72 |
| ATM, ERCC1, PAR, XPF | 7.36E−04 | | 0.29 | 1.00 | 1.00 | 0.57 | 0.37 | 2.33 |
| ATM, PAR, XPF, pH2AX | 7.36E−04 | | 0.29 | 1.00 | 1.00 | 0.57 | 0.37 | 2.33 |
| ERCC1, PAR, XPF, pH2AX | 7.36E−04 | | 0.29 | 1.00 | 1.00 | 0.57 | 0.37 | 2.33 |
| BRCA1, ERCC1, PAR, pH2AX | 8.77E−04 | | 0.86 | 0.55 | 0.67 | 0.79 | 0.29 | 3.11 |
| ATM, BRCA1, PAR, pH2AX | 1.16E−03 | | 0.95 | 0.45 | 0.65 | 0.90 | 0.29 | 6.45 |
| BRCA1, PAR, pMK2, pH2AX | 1.16E−03 | | 0.95 | 0.45 | 0.65 | 0.90 | 0.29 | 6.45 |
| ATM, PAR, RAD51, pH2AX | 1.52E−03 | | 0.45 | 0.95 | 0.90 | 0.63 | 0.30 | 2.45 |
| PAR, RAD51, pMK2, pH2AX | 1.52E−03 | | 0.45 | 0.95 | 0.90 | 0.63 | 0.30 | 2.45 |
| ATM, PAR, p53, pMK2 | 1.60E−03 | | 0.52 | 0.85 | 0.79 | 0.63 | 0.32 | 2.12 |
| ATM, p53, pMK2, pH2AX | 1.66E−03 | | 0.61 | 0.85 | 0.82 | 0.65 | 0.28 | 2.38 |
| ATM, ERCC1, PAR, RAD51 | 1.67E−03 | | 0.30 | 1.00 | 1.00 | 0.59 | 0.35 | 2.43 |
| ERCC1, PAR, RAD51, pH2AX | 1.67E−03 | | 0.30 | 1.00 | 1.00 | 0.59 | 0.35 | 2.43 |
| ERCC1, PAR, RAD51, pMK2 | 1.67E−03 | | 0.30 | 1.00 | 1.00 | 0.59 | 0.35 | 2.43 |
| ATM, RAD51, p53, pMK2 | 2.02E−03 | | 0.45 | 0.85 | 0.77 | 0.59 | 0.36 | 1.86 |
| ATM, ERCC1, RAD51, p53 | 2.29E−03 | | 0.27 | 1.00 | 1.00 | 0.56 | 0.38 | 2.25 |
| ERCC1, RAD51, p53, pMK2 | 2.29E−03 | | 0.27 | 1.00 | 1.00 | 0.56 | 0.38 | 2.25 |
| BRCA1, ERCC1, pMK2, pH2AX | 2.41E−03 | | 0.57 | 0.80 | 0.76 | 0.62 | 0.33 | 1.99 |
| ATM, BRCA1, ERCC1, pH2AX | 2.51E−03 | | 0.83 | 0.50 | 0.66 | 0.71 | 0.33 | 2.29 |
| ATM, BRCA1, pMK2, pH2AX | 2.62E−03 | | 0.96 | 0.40 | 0.65 | 0.89 | 0.30 | 5.82 |
| BRCA1, ERCC1, PAR, pMK2 | 2.71E−03 | | 0.57 | 0.85 | 0.80 | 0.65 | 0.29 | 2.31 |
| ATM, BRCA1, PAR, pMK2 | 3.32E−03 | | 0.67 | 0.80 | 0.78 | 0.70 | 0.27 | 2.56 |
| ATM, BRCA1, ERCC1, PAR | 4.14E−03 | | 0.52 | 0.90 | 0.85 | 0.64 | 0.29 | 2.37 |
| ATM, ERCC1, p53, pH2AX | 4.72E−03 | | 0.78 | 0.65 | 0.72 | 0.72 | 0.28 | 2.59 |
| ATM, PAR, p53, pH2AX | 6.04E−03 | | 0.67 | 0.75 | 0.74 | 0.68 | 0.29 | 2.32 |
| ATM, ERCC1, PAR, p53 | 6.28E−03 | | 0.81 | 0.70 | 0.74 | 0.78 | 0.24 | 3.33 |
| ATM, ERCC1, p53, pMK2 | 8.80E−03 | | 0.61 | 0.80 | 0.78 | 0.64 | 0.30 | 2.16 |
| ATM, BRCA1, ERCC1, pMK2 | 9.97E−03 | | 0.78 | 0.65 | 0.72 | 0.72 | 0.28 | 2.59 |
| ERCC1, PAR, p53, pMK2 | 1.05E−02 | | 0.67 | 0.75 | 0.74 | 0.68 | 0.29 | 2.32 |
| ERCC1, p53, pMK2, pH2AX | 1.18E−02 | | 0.70 | 0.65 | 0.70 | 0.65 | 0.33 | 1.99 |
| ATM, PAR, RAD51, pMK2 | 1.20E−02 | | 0.75 | 0.80 | 0.79 | 0.76 | 0.22 | 3.32 |
| ERCC1, PAR, p53, pH2AX | 1.47E−02 | | 0.33 | 0.95 | 0.88 | 0.58 | 0.37 | 2.06 |
| PAR, p53, pMK2, pH2AX | 1.47E−02 | | 0.33 | 0.95 | 0.88 | 0.58 | 0.37 | 2.06 |
| ATM, ERCC1, pMK2, pH2AX | 1.56E−02 | | 0.65 | 0.75 | 0.75 | 0.65 | 0.30 | 2.16 |
| ATM, ERCC1, PAR, pH2AX | 1.96E−02 | | 0.81 | 0.60 | 0.68 | 0.75 | 0.29 | 2.72 |
| ATM, ERCC1, RAD51, pMK2 | 2.94E−02 | | 0.82 | 0.55 | 0.67 | 0.73 | 0.31 | 2.50 |
| ATM, ERCC1, PAR, pMK2 | 3.05E−02 | | 0.29 | 1.00 | 1.00 | 0.57 | 0.37 | 2.33 |
| ATM, PAR, pMK2, pH2AX | 3.22E−02 | | 0.52 | 0.75 | 0.69 | 0.60 | 0.37 | 1.72 |
| ERCC1, PAR, pMK2, pH2AX | 4.92E−02 | | 0.71 | 0.60 | 0.65 | 0.67 | 0.34 | 1.96 |

TABLE 6
One HNCMARKER Probability Analysis on Survival for Head and Neck Cancer
After Chemotherapy and Radiation

| Markers | pval | AUC | Sens | Spec | PosPow | NegPow | AER | RelRisk |
|---|---|---|---|---|---|---|---|---|
| FANCD2 | 1.99E−03 | 0.73 | 0.92 | 0.55 | 0.69 | 0.86 | 0.26 | 4.81 |
| XPF | 4.22E−03 | 0.69 | 0.50 | 0.86 | 0.80 | 0.61 | 0.33 | 2.07 |
| BRCA1 | 1.01E−02 | 0.75 | 0.87 | 0.55 | 0.67 | 0.80 | 0.29 | 3.33 |
| RAD51 | 3.59E−02 | 0.68 | 0.91 | 0.48 | 0.65 | 0.83 | 0.30 | 3.87 |
| p53 | 1.14E−01 | 0.70 | 0.65 | 0.62 | 0.65 | 0.62 | 0.36 | 1.71 |
| ATM | 1.67E−01 | 0.65 | 0.74 | 0.52 | 0.63 | 0.65 | 0.36 | 1.78 |
| PAR | 3.26E−01 | 0.65 | 0.86 | 0.33 | 0.58 | 0.70 | 0.40 | 1.92 |
| pH2AX | 8.84E−01 | 0.50 | 0.88 | 0.14 | 0.54 | 0.50 | 0.47 | 1.08 |
| pMK2 | 9.17E−01 | 0.57 | 0.74 | 0.39 | 0.55 | 0.60 | 0.43 | 1.37 |
| ERCC1 | 9.24E−01 | 0.56 | 0.43 | 0.67 | 0.59 | 0.52 | 0.45 | 1.22 |

TABLE 7
Two HNCMARKER Probability Analysis on Survival for Head and Neck Cancer
patients treated with chemotherapy and radiation

| Markers | | pval | AUC | Sens | Spec | PosPow | NegPow | AER | RelRisk |
|---|---|---|---|---|---|---|---|---|---|
| BRCA1 | RAD51 | 1.88E−04 | 0.80 | 0.91 | 0.71 | 0.77 | 0.88 | 0.19 | 6.54 |
| FANCD2 | RAD51 | 2.05E−04 | 0.82 | 0.95 | 0.67 | 0.75 | 0.93 | 0.19 | 11.25 |
| BRCA1 | FANCD2 | 2.69E−04 | 0.82 | 0.91 | 0.64 | 0.72 | 0.88 | 0.22 | 5.79 |
| ATM | FANCD2 | 2.87E−04 | 0.81 | 0.96 | 0.62 | 0.73 | 0.93 | 0.20 | 10.27 |
| ATM | XPF | 5.35E−04 | 0.73 | 0.65 | 0.86 | 0.83 | 0.69 | 0.25 | 2.71 |
| XPF | p53 | 8.52E−04 | 0.73 | 0.61 | 0.86 | 0.82 | 0.67 | 0.27 | 2.47 |
| FANCD2 | pMK2 | 1.15E−03 | 0.83 | 0.96 | 0.55 | 0.69 | 0.92 | 0.24 | 8.94 |
| FANCD2 | XPF | 1.24E−03 | 0.79 | 0.79 | 0.68 | 0.73 | 0.75 | 0.26 | 2.92 |
| BRCA1 | XPF | 1.85E−03 | 0.77 | 0.74 | 0.73 | 0.74 | 0.73 | 0.27 | 2.71 |
| ERCC1 | FANCD2 | 2.32E−03 | 0.86 | 0.96 | 0.52 | 0.69 | 0.92 | 0.25 | 8.25 |
| FANCD2 | p53 | 2.32E−03 | 0.85 | 0.96 | 0.52 | 0.69 | 0.92 | 0.25 | 8.25 |
| BRCA1 | p53 | 2.89E−03 | 0.78 | 0.87 | 0.62 | 0.71 | 0.81 | 0.25 | 3.81 |
| XPF | pMK2 | 3.16E−03 | 0.75 | 0.61 | 0.82 | 0.78 | 0.67 | 0.29 | 2.33 |
| ERCC1 | XPF | 5.66E−03 | 0.73 | 0.57 | 0.86 | 0.81 | 0.64 | 0.30 | 2.28 |
| BRCA1 | pH2AX | 5.89E−03 | 0.72 | 0.91 | 0.48 | 0.66 | 0.83 | 0.30 | 3.94 |
| RAD51 | p53 | 6.12E−03 | 0.76 | 0.86 | 0.62 | 0.70 | 0.81 | 0.26 | 3.75 |
| ERCC1 | RAD51 | 1.26E−02 | 0.70 | 0.86 | 0.57 | 0.68 | 0.80 | 0.28 | 3.39 |
| RAD51 | pMK2 | 1.63E−02 | 0.70 | 0.86 | 0.62 | 0.70 | 0.81 | 0.26 | 3.75 |
| BRCA1 | ERCC1 | 1.83E−02 | 0.75 | 0.83 | 0.57 | 0.68 | 0.75 | 0.30 | 2.71 |
| ATM | BRCA1 | 2.03E−02 | 0.76 | 0.83 | 0.52 | 0.66 | 0.73 | 0.32 | 2.46 |
| FANCD2 | pH2AX | 2.22E−02 | 0.71 | 0.88 | 0.48 | 0.66 | 0.77 | 0.31 | 2.84 |
| RAD51 | XPF | 2.43E−02 | 0.75 | 0.73 | 0.67 | 0.70 | 0.70 | 0.30 | 2.32 |
| BRCA1 | pMK2 | 2.79E−02 | 0.72 | 0.83 | 0.55 | 0.66 | 0.75 | 0.31 | 2.62 |
| RAD51 | pH2AX | 3.59E−02 | 0.72 | 0.91 | 0.48 | 0.65 | 0.83 | 0.30 | 3.87 |
| ATM | p53 | 4.06E−02 | 0.73 | 0.70 | 0.67 | 0.70 | 0.67 | 0.32 | 2.09 |
| PAR | RAD51 | 4.90E−02 | 0.76 | 0.95 | 0.43 | 0.61 | 0.90 | 0.32 | 6.13 |
| ATM | RAD51 | 5.37E−02 | 0.75 | 0.77 | 0.57 | 0.65 | 0.71 | 0.33 | 2.22 |
| XPF | pH2AX | 9.49E−02 | 0.67 | 0.71 | 0.52 | 0.63 | 0.61 | 0.38 | 1.62 |
| BRCA1 | PAR | 1.07E−01 | 0.75 | 0.86 | 0.43 | 0.60 | 0.75 | 0.36 | 2.40 |
| FANCD2 | PAR | 1.76E−01 | 0.74 | 0.86 | 0.38 | 0.59 | 0.73 | 0.37 | 2.18 |
| p53 | pMK2 | 2.13E−01 | 0.70 | 0.78 | 0.52 | 0.64 | 0.69 | 0.34 | 2.06 |
| ATM | ERCC1 | 2.36E−01 | 0.73 | 0.78 | 0.52 | 0.64 | 0.69 | 0.34 | 2.06 |
| ATM | pMK2 | 2.91E−01 | 0.70 | 0.65 | 0.62 | 0.65 | 0.62 | 0.36 | 1.71 |
| ATM | PAR | 2.94E−01 | 0.71 | 0.90 | 0.29 | 0.56 | 0.75 | 0.40 | 2.24 |
| ERCC1 | p53 | 2.96E−01 | 0.70 | 0.52 | 0.76 | 0.71 | 0.59 | 0.36 | 1.73 |
| PAR | XPF | 3.02E−01 | 0.72 | 0.86 | 0.29 | 0.56 | 0.67 | 0.42 | 1.68 |
| ERCC1 | PAR | 3.58E−01 | 0.59 | 0.86 | 0.33 | 0.56 | 0.70 | 0.40 | 1.88 |
| PAR | p53 | 3.58E−01 | 0.74 | 0.86 | 0.33 | 0.56 | 0.70 | 0.40 | 1.88 |
| pMK2 | pH2AX | 4.16E−01 | 0.59 | 0.91 | 0.29 | 0.58 | 0.75 | 0.39 | 2.33 |
| PAR | pH2AX | 4.25E−01 | 0.61 | 0.86 | 0.38 | 0.59 | 0.73 | 0.37 | 2.18 |
| p53 | pH2AX | 4.88E−01 | 0.55 | 0.91 | 0.14 | 0.54 | 0.60 | 0.45 | 1.35 |
| ATM | pH2AX | 5.02E−01 | 0.59 | 0.87 | 0.19 | 0.54 | 0.57 | 0.45 | 1.26 |
| ERCC1 | pH2AX | 5.50E−01 | 0.50 | 0.87 | 0.24 | 0.56 | 0.63 | 0.43 | 1.48 |
| ERCC1 | pMK2 | 5.85E−01 | 0.63 | 0.65 | 0.52 | 0.60 | 0.58 | 0.41 | 1.43 |
| PAR | pMK2 | 9.89E−01 | 0.65 | 0.81 | 0.29 | 0.53 | 0.60 | 0.45 | 1.33 |

TABLE 8
Three HNCMARKER Probability Analysis on Survival for Head and Neck
Cancer patients treated with chemotherapy and radiation

| Markers | | | pval | AUC | Sens | Spec | PosPow | NegPow | AER | RelRisk |
|---|---|---|---|---|---|---|---|---|---|---|
| BRCA1 | RAD51 | p53 | 3.40E−05 | 0.84 | 0.91 | 0.76 | 0.80 | 0.89 | 0.16 | 7.20 |
| FANCD2 | RAD51 | XPF | 6.76E−05 | 0.86 | 0.95 | 0.71 | 0.78 | 0.94 | 0.16 | 12.44 |
| ATM | XPF | pMK2 | 1.20E−04 | 0.76 | 0.70 | 0.86 | 0.84 | 0.72 | 0.23 | 3.01 |
| BRCA1 | FANCD2 | RAD51 | 1.22E−04 | 0.84 | 0.91 | 0.71 | 0.77 | 0.88 | 0.19 | 6.54 |
| ATM | BRCA1 | FANCD2 | 1.26E−04 | 0.81 | 0.91 | 0.67 | 0.75 | 0.88 | 0.20 | 6.00 |
| ATM | FANCD2 | XPF | 1.26E−04 | 0.85 | 0.91 | 0.67 | 0.75 | 0.88 | 0.20 | 6.00 |
| FANCD2 | XPF | pMK2 | 1.34E−04 | 0.87 | 0.96 | 0.68 | 0.76 | 0.94 | 0.18 | 12.14 |
| ATM | BRCA1 | RAD51 | 1.88E−04 | 0.81 | 0.91 | 0.76 | 0.80 | 0.89 | 0.16 | 7.20 |
| ERCC1 | FANCD2 | RAD51 | 2.05E−04 | 0.85 | 0.95 | 0.67 | 0.75 | 0.93 | 0.19 | 11.25 |
| FANCD2 | RAD51 | p53 | 2.05E−04 | 0.86 | 0.95 | 0.67 | 0.75 | 0.93 | 0.19 | 11.25 |
| BRCA1 | FANCD2 | XPF | 2.45E−04 | 0.84 | 0.87 | 0.68 | 0.74 | 0.83 | 0.22 | 4.44 |
| BRCA1 | FANCD2 | pMK2 | 2.69E−04 | 0.81 | 0.91 | 0.64 | 0.72 | 0.88 | 0.22 | 5.79 |
| ATM | FANCD2 | p53 | 2.87E−04 | 0.83 | 0.96 | 0.62 | 0.73 | 0.93 | 0.20 | 10.27 |
| ERCC1 | FANCD2 | XPF | 2.87E−04 | 0.87 | 0.96 | 0.62 | 0.73 | 0.93 | 0.20 | 10.27 |
| FANCD2 | XPF | p53 | 2.87E−04 | 0.86 | 0.96 | 0.62 | 0.73 | 0.93 | 0.20 | 10.27 |
| BRCA1 | RAD51 | XPF | 4.89E−04 | 0.81 | 0.82 | 0.76 | 0.78 | 0.80 | 0.21 | 3.91 |
| ATM | XPF | p53 | 5.35E−04 | 0.75 | 0.65 | 0.86 | 0.83 | 0.69 | 0.25 | 2.71 |
| BRCA1 | ERCC1 | FANCD2 | 5.56E−04 | 0.83 | 0.91 | 0.62 | 0.72 | 0.87 | 0.23 | 5.43 |
| BRCA1 | FANCD2 | p53 | 5.56E−04 | 0.81 | 0.91 | 0.62 | 0.72 | 0.87 | 0.23 | 5.43 |
| FANCD2 | p53 | pH2AX | 7.05E−04 | 0.79 | 0.96 | 0.57 | 0.71 | 0.92 | 0.23 | 9.23 |
| ATM | FANCD2 | RAD51 | 7.90E−04 | 0.85 | 0.95 | 0.62 | 0.72 | 0.93 | 0.21 | 10.14 |
| FANCD2 | RAD51 | pMK2 | 7.90E−04 | 0.82 | 0.95 | 0.62 | 0.72 | 0.93 | 0.21 | 10.14 |
| FANCD2 | RAD51 | pH2AX | 7.90E−04 | 0.84 | 0.95 | 0.62 | 0.72 | 0.93 | 0.21 | 10.14 |
| BRCA1 | RAD51 | pMK2 | 7.99E−04 | 0.79 | 0.91 | 0.67 | 0.74 | 0.88 | 0.21 | 5.93 |
| ATM | ERCC1 | FANCD2 | 1.04E−03 | 0.87 | 0.96 | 0.57 | 0.71 | 0.92 | 0.23 | 9.23 |
| ATM | FANCD2 | pMK2 | 1.04E−03 | 0.83 | 0.96 | 0.57 | 0.71 | 0.92 | 0.23 | 9.23 |
| FANCD2 | PAR | RAD51 | 1.10E−03 | 0.87 | 0.95 | 0.67 | 0.73 | 0.93 | 0.20 | 10.96 |
| RAD51 | XPF | p53 | 1.30E−03 | 0.80 | 0.73 | 0.76 | 0.76 | 0.73 | 0.26 | 2.79 |
| BRCA1 | FANCD2 | pH2AX | 1.32E−03 | 0.78 | 0.87 | 0.62 | 0.71 | 0.81 | 0.25 | 3.81 |
| ERCC1 | FANCD2 | pH2AX | 1.59E−03 | 0.78 | 0.96 | 0.52 | 0.69 | 0.92 | 0.25 | 8.25 |
| BRCA1 | ERCC1 | p53 | 1.97E−03 | 0.78 | 0.87 | 0.67 | 0.74 | 0.82 | 0.23 | 4.20 |
| BRCA1 | RAD51 | pH2AX | 2.06E−03 | 0.81 | 0.91 | 0.57 | 0.69 | 0.86 | 0.26 | 4.83 |
| ATM | FANCD2 | pH2AX | 2.32E−03 | 0.77 | 0.96 | 0.52 | 0.69 | 0.92 | 0.25 | 8.25 |
| ERCC1 | FANCD2 | p53 | 2.32E−03 | 0.88 | 0.96 | 0.52 | 0.69 | 0.92 | 0.25 | 8.25 |
| ERCC1 | FANCD2 | pMK2 | 2.32E−03 | 0.85 | 0.96 | 0.57 | 0.71 | 0.92 | 0.23 | 9.23 |
| FANCD2 | p53 | pMK2 | 2.32E−03 | 0.84 | 0.96 | 0.52 | 0.69 | 0.92 | 0.25 | 8.25 |
| ATM | ERCC1 | XPF | 2.96E−03 | 0.75 | 0.65 | 0.81 | 0.79 | 0.68 | 0.27 | 2.47 |
| XPF | p53 | pMK2 | 3.26E−03 | 0.77 | 0.65 | 0.81 | 0.79 | 0.68 | 0.27 | 2.47 |
| BRCA1 | XPF | p53 | 3.30E−03 | 0.78 | 0.74 | 0.71 | 0.74 | 0.71 | 0.27 | 2.59 |
| FANCD2 | pMK2 | pH2AX | 4.81E−03 | 0.81 | 0.96 | 0.48 | 0.67 | 0.91 | 0.27 | 7.33 |
| ERCC1 | XPF | p53 | 5.63E−03 | 0.78 | 0.52 | 0.86 | 0.80 | 0.62 | 0.32 | 2.11 |
| ATM | BRCA1 | pH2AX | 5.89E−03 | 0.73 | 0.91 | 0.48 | 0.66 | 0.83 | 0.30 | 3.94 |
| BRCA1 | p53 | pH2AX | 5.89E−03 | 0.74 | 0.91 | 0.48 | 0.66 | 0.83 | 0.30 | 3.94 |
| BRCA1 | pMK2 | pH2AX | 5.89E−03 | 0.71 | 0.91 | 0.52 | 0.68 | 0.85 | 0.27 | 4.40 |
| BRCA1 | FANCD2 | PAR | 5.92E−03 | 0.84 | 0.90 | 0.52 | 0.66 | 0.85 | 0.29 | 4.26 |
| ERCC1 | RAD51 | p53 | 6.12E−03 | 0.76 | 0.86 | 0.62 | 0.70 | 0.81 | 0.26 | 3.75 |
| ATM | BRCA1 | ERCC1 | 6.48E−03 | 0.78 | 0.87 | 0.57 | 0.69 | 0.80 | 0.27 | 3.45 |
| FANCD2 | XPF | pH2AX | 6.73E−03 | 0.76 | 0.83 | 0.52 | 0.67 | 0.73 | 0.31 | 2.50 |
| BRCA1 | ERCC1 | RAD51 | 6.94E−03 | 0.83 | 0.82 | 0.67 | 0.72 | 0.78 | 0.26 | 3.24 |
| BRCA1 | XPF | pMK2 | 9.52E−03 | 0.79 | 0.78 | 0.68 | 0.72 | 0.75 | 0.27 | 2.88 |
| ERCC1 | XPF | pMK2 | 9.63E−03 | 0.77 | 0.61 | 0.81 | 0.78 | 0.65 | 0.30 | 2.25 |
| ATM | RAD51 | XPF | 1.01E−02 | 0.79 | 0.77 | 0.67 | 0.71 | 0.74 | 0.28 | 2.69 |
| BRCA1 | PAR | XPF | 1.26E−02 | 0.78 | 0.86 | 0.52 | 0.64 | 0.79 | 0.31 | 3.00 |
| RAD51 | XPF | pH2AX | 1.32E−02 | 0.78 | 0.86 | 0.57 | 0.68 | 0.80 | 0.28 | 3.39 |
| FANCD2 | PAR | p53 | 1.52E−02 | 0.86 | 0.90 | 0.52 | 0.66 | 0.85 | 0.29 | 4.26 |
| FANCD2 | PAR | XPF | 1.60E−02 | 0.79 | 0.86 | 0.48 | 0.63 | 0.77 | 0.33 | 2.74 |
| BRCA1 | ERCC1 | XPF | 1.67E−02 | 0.78 | 0.70 | 0.67 | 0.70 | 0.67 | 0.32 | 2.09 |
| BRCA1 | PAR | pH2AX | 2.02E−02 | 0.73 | 0.90 | 0.52 | 0.66 | 0.85 | 0.29 | 4.26 |
| ATM | BRCA1 | p53 | 2.03E−02 | 0.80 | 0.83 | 0.52 | 0.66 | 0.73 | 0.32 | 2.46 |
| PAR | RAD51 | p53 | 2.11E−02 | 0.83 | 0.95 | 0.52 | 0.66 | 0.92 | 0.27 | 7.86 |
| BRCA1 | PAR | RAD51 | 2.23E−02 | 0.86 | 0.85 | 0.57 | 0.65 | 0.80 | 0.29 | 3.27 |
| RAD51 | XPF | pMK2 | 2.27E−02 | 0.77 | 0.77 | 0.62 | 0.68 | 0.72 | 0.30 | 2.45 |
| BRCA1 | p53 | pMK2 | 2.48E−02 | 0.76 | 0.83 | 0.57 | 0.68 | 0.75 | 0.30 | 2.71 |
| PAR | RAD51 | XPF | 2.78E−02 | 0.82 | 0.90 | 0.57 | 0.67 | 0.86 | 0.27 | 4.67 |
| ERCC1 | RAD51 | pH2AX | 2.90E−02 | 0.71 | 0.91 | 0.43 | 0.63 | 0.82 | 0.33 | 3.44 |
| XPF | pMK2 | pH2AX | 2.93E−02 | 0.74 | 0.83 | 0.52 | 0.66 | 0.73 | 0.32 | 2.46 |
| ATM | XPF | pH2AX | 3.22E−02 | 0.71 | 0.78 | 0.57 | 0.67 | 0.71 | 0.32 | 2.27 |
| ERCC1 | RAD51 | pMK2 | 3.41E−02 | 0.75 | 0.82 | 0.62 | 0.69 | 0.76 | 0.28 | 2.94 |
| RAD51 | p53 | pH2AX | 3.59E−02 | 0.77 | 0.91 | 0.43 | 0.63 | 0.82 | 0.33 | 3.44 |
| BRCA1 | XPF | pH2AX | 3.65E−02 | 0.76 | 0.83 | 0.48 | 0.63 | 0.71 | 0.34 | 2.22 |
| ATM | FANCD2 | PAR | 3.67E−02 | 0.85 | 0.90 | 0.52 | 0.66 | 0.85 | 0.29 | 4.26 |
| ERCC1 | FANCD2 | PAR | 3.67E−02 | 0.85 | 0.90 | 0.52 | 0.66 | 0.85 | 0.29 | 4.26 |
| FANCD2 | PAR | pMK2 | 3.67E−02 | 0.83 | 0.90 | 0.52 | 0.66 | 0.85 | 0.29 | 4.26 |
| ATM | BRCA1 | XPF | 3.74E−02 | 0.77 | 0.74 | 0.67 | 0.71 | 0.70 | 0.30 | 2.36 |
| BRCA1 | ERCC1 | pH2AX | 4.49E−02 | 0.71 | 0.87 | 0.43 | 0.63 | 0.75 | 0.34 | 2.50 |
| RAD51 | p53 | pMK2 | 4.55E−02 | 0.77 | 0.82 | 0.62 | 0.69 | 0.76 | 0.28 | 2.94 |
| ATM | PAR | RAD51 | 4.90E−02 | 0.82 | 0.95 | 0.48 | 0.63 | 0.91 | 0.29 | 6.97 |
| PAR | RAD51 | pMK2 | 4.90E−02 | 0.78 | 0.95 | 0.43 | 0.61 | 0.90 | 0.32 | 6.13 |
| BRCA1 | ERCC1 | PAR | 4.95E−02 | 0.73 | 0.86 | 0.48 | 0.62 | 0.77 | 0.33 | 2.69 |
| ATM | BRCA1 | pMK2 | 5.07E−02 | 0.75 | 0.83 | 0.52 | 0.66 | 0.73 | 0.32 | 2.46 |
| BRCA1 | PAR | p53 | 5.94E−02 | 0.78 | 0.81 | 0.52 | 0.63 | 0.73 | 0.33 | 2.36 |
| FANCD2 | PAR | pH2AX | 6.00E−02 | 0.73 | 0.95 | 0.38 | 0.62 | 0.89 | 0.33 | 5.56 |
| ERCC1 | p53 | pMK2 | 6.59E−02 | 0.72 | 0.78 | 0.67 | 0.72 | 0.74 | 0.27 | 2.74 |
| ATM | RAD51 | pH2AX | 6.63E−02 | 0.76 | 0.91 | 0.43 | 0.63 | 0.82 | 0.33 | 3.44 |
| ATM | BRCA1 | PAR | 7.11E−02 | 0.78 | 0.81 | 0.48 | 0.61 | 0.71 | 0.36 | 2.13 |
| XPF | p53 | pH2AX | 8.16E−02 | 0.70 | 0.74 | 0.52 | 0.63 | 0.65 | 0.36 | 1.78 |
| ERCC1 | PAR | XPF | 8.65E−02 | 0.73 | 0.86 | 0.38 | 0.58 | 0.73 | 0.38 | 2.13 |
| ATM | p53 | pMK2 | 8.73E−02 | 0.76 | 0.70 | 0.71 | 0.73 | 0.68 | 0.30 | 2.29 |
| BRCA1 | ERCC1 | pMK2 | 8.77E−02 | 0.75 | 0.78 | 0.57 | 0.67 | 0.71 | 0.32 | 2.27 |
| ATM | ERCC1 | RAD51 | 8.99E−02 | 0.78 | 0.82 | 0.52 | 0.64 | 0.73 | 0.33 | 2.41 |
| ATM | RAD51 | p53 | 8.99E−02 | 0.80 | 0.82 | 0.52 | 0.64 | 0.73 | 0.33 | 2.41 |
| RAD51 | pMK2 | pH2AX | 9.06E−02 | 0.75 | 0.86 | 0.43 | 0.61 | 0.75 | 0.35 | 2.45 |
| ATM | ERCC1 | p53 | 9.95E−02 | 0.82 | 0.78 | 0.62 | 0.69 | 0.72 | 0.30 | 2.49 |
| BRCA1 | PAR | pMK2 | 1.07E−01 | 0.74 | 0.86 | 0.43 | 0.60 | 0.75 | 0.36 | 2.40 |
| ERCC1 | XPF | pH2AX | 1.24E−01 | 0.69 | 0.78 | 0.48 | 0.62 | 0.67 | 0.36 | 1.86 |
| ATM | RAD51 | pMK2 | 1.25E−01 | 0.77 | 0.73 | 0.62 | 0.67 | 0.68 | 0.33 | 2.11 |
| PAR | XPF | pH2AX | 1.35E−01 | 0.71 | 0.86 | 0.38 | 0.59 | 0.73 | 0.37 | 2.18 |
| ERCC1 | PAR | RAD51 | 1.47E−01 | 0.76 | 0.90 | 0.43 | 0.60 | 0.82 | 0.34 | 3.30 |
| PAR | XPF | p53 | 1.51E−01 | 0.79 | 0.86 | 0.38 | 0.58 | 0.73 | 0.38 | 2.13 |
| ATM | PAR | p53 | 2.54E−01 | 0.78 | 0.90 | 0.33 | 0.58 | 0.78 | 0.38 | 2.59 |
| PAR | RAD51 | pH2AX | 2.74E−01 | 0.81 | 0.85 | 0.48 | 0.61 | 0.77 | 0.34 | 2.63 |
| ERCC1 | RAD51 | XPF | 2.78E−01 | 0.76 | 0.64 | 0.57 | 0.61 | 0.60 | 0.40 | 1.52 |
| PAR | XPF | pMK2 | 2.88E−01 | 0.76 | 0.86 | 0.33 | 0.56 | 0.70 | 0.40 | 1.88 |
| ATM | PAR | pMK2 | 2.94E−01 | 0.72 | 0.90 | 0.29 | 0.56 | 0.75 | 0.40 | 2.24 |
| ERCC1 | PAR | p53 | 3.58E−01 | 0.71 | 0.86 | 0.33 | 0.56 | 0.70 | 0.40 | 1.88 |
| ATM | PAR | XPF | 4.30E−01 | 0.77 | 0.76 | 0.43 | 0.57 | 0.64 | 0.40 | 1.60 |
| ERCC1 | PAR | pH2AX | 4.51E−01 | 0.60 | 0.86 | 0.38 | 0.58 | 0.73 | 0.38 | 2.13 |
| PAR | pMK2 | pH2AX | 4.51E−01 | 0.64 | 0.86 | 0.38 | 0.58 | 0.73 | 0.38 | 2.13 |
| PAR | p53 | pMK2 | 5.11E−01 | 0.75 | 0.86 | 0.33 | 0.56 | 0.70 | 0.40 | 1.88 |
| ATM | p53 | pH2AX | 5.52E−01 | 0.63 | 0.91 | 0.14 | 0.54 | 0.60 | 0.45 | 1.35 |
| ATM | PAR | pH2AX | 5.64E−01 | 0.70 | 0.81 | 0.38 | 0.57 | 0.67 | 0.40 | 1.70 |
| PAR | p53 | pH2AX | 5.64E−01 | 0.71 | 0.81 | 0.38 | 0.57 | 0.67 | 0.40 | 1.70 |
| ATM | ERCC1 | pH2AX | 6.14E−01 | 0.65 | 0.87 | 0.24 | 0.56 | 0.63 | 0.43 | 1.48 |
| ATM | ERCC1 | PAR | 6.19E−01 | 0.73 | 0.86 | 0.29 | 0.55 | 0.67 | 0.43 | 1.64 |
| ERCC1 | PAR | pMK2 | 6.19E−01 | 0.63 | 0.86 | 0.29 | 0.55 | 0.67 | 0.43 | 1.64 |
| p53 | pMK2 | pH2AX | 7.64E−01 | 0.63 | 0.91 | 0.24 | 0.57 | 0.71 | 0.41 | 1.99 |
| ERCC1 | pMK2 | pH2AX | 8.15E−01 | 0.59 | 0.87 | 0.33 | 0.59 | 0.70 | 0.39 | 1.96 |
| ERCC1 | p53 | pH2AX | 9.16E−01 | 0.55 | 0.83 | 0.24 | 0.54 | 0.56 | 0.45 | 1.22 |
| ATM | ERCC1 | pMK2 | 9.37E−01 | 0.74 | 0.70 | 0.48 | 0.59 | 0.59 | 0.41 | 1.44 |
| ATM | pMK2 | pH2AX | 9.47E−01 | 0.64 | 0.83 | 0.29 | 0.56 | 0.60 | 0.43 | 1.40 |

TABLE 9
Four HNCMARKER Probability Analysis on Survival for Head and Neck Cancer patients treated with chemotherapy and radiation

| Markers | | | | pval | AUC | Sens | Spec | PosPow | NegPow | AER | RelRisk | RelRisk.CI.lower |
|---|---|---|---|---|---|---|---|---|---|---|---|---|
| ATM | FANCD2 | RAD51 | XPF | 1.17E−05 | 0.87 | 0.95 | 0.81 | 0.84 | 0.94 | 0.12 | 15.12 | 2.23 |
| BRCA1 | ERCC1 | FANCD2 | RAD51 | 2.05E−05 | 0.87 | 0.91 | 0.76 | 0.80 | 0.89 | 0.16 | 7.20 | 1.92 |
| BRCA1 | FANCD2 | RAD51 | p53 | 2.05E−05 | 0.86 | 0.91 | 0.76 | 0.80 | 0.89 | 0.16 | 7.20 | 1.92 |
| ATM | BRCA1 | RAD51 | p53 | 3.40E−05 | 0.84 | 0.91 | 0.76 | 0.80 | 0.89 | 0.16 | 7.20 | 1.92 |
| BRCA1 | RAD51 | p53 | pMK2 | 3.40E−05 | 0.83 | 0.91 | 0.76 | 0.80 | 0.89 | 0.16 | 7.20 | 1.92 |
| FANCD2 | RAD51 | XPF | pMK2 | 6.76E−05 | 0.85 | 0.95 | 0.71 | 0.78 | 0.94 | 0.16 | 12.44 | 1.85 |
| BRCA1 | FANCD2 | PAR | RAD51 | 9.46E−05 | 0.89 | 0.95 | 0.71 | 0.76 | 0.94 | 0.17 | 12.16 | 1.80 |
| BRCA1 | FANCD2 | RAD51 | XPF | 9.50E−05 | 0.86 | 0.86 | 0.76 | 0.79 | 0.84 | 0.19 | 5.01 | 1.74 |
| BRCA1 | FANCD2 | RAD51 | pH2AX | 9.50E−05 | 0.84 | 0.86 | 0.76 | 0.79 | 0.84 | 0.19 | 5.01 | 1.74 |
| ATM | XPF | p53 | pMK2 | 1.20E−04 | 0.77 | 0.70 | 0.86 | 0.84 | 0.72 | 0.23 | 3.01 | 1.56 |
| ATM | BRCA1 | FANCD2 | RAD51 | 1.22E−04 | 0.85 | 0.91 | 0.71 | 0.77 | 0.88 | 0.19 | 6.54 | 1.75 |
| BRCA1 | FANCD2 | RAD51 | pMK2 | 1.22E−04 | 0.84 | 0.91 | 0.71 | 0.77 | 0.88 | 0.19 | 6.54 | 1.75 |
| ATM | ERCC1 | FANCD2 | XPF | 1.26E−04 | 0.87 | 0.91 | 0.67 | 0.75 | 0.88 | 0.20 | 6.00 | 1.61 |
| ATM | BRCA1 | FANCD2 | p53 | 1.26E−04 | 0.81 | 0.91 | 0.67 | 0.75 | 0.88 | 0.20 | 6.00 | 1.61 |
| ATM | FANCD2 | XPF | p53 | 1.26E−04 | 0.85 | 0.91 | 0.67 | 0.75 | 0.88 | 0.20 | 6.00 | 1.61 |
| ATM | FANCD2 | XPF | pMK2 | 1.26E−04 | 0.86 | 0.91 | 0.71 | 0.78 | 0.88 | 0.18 | 6.61 | 1.77 |
| BRCA1 | ERCC1 | FANCD2 | p53 | 1.26E−04 | 0.84 | 0.91 | 0.67 | 0.75 | 0.88 | 0.20 | 6.00 | 1.61 |
| ATM | ERCC1 | XPF | pMK2 | 1.55E−04 | 0.79 | 0.74 | 0.86 | 0.85 | 0.75 | 0.20 | 3.40 | 1.66 |
| ATM | BRCA1 | ERCC1 | RAD51 | 1.88E−04 | 0.83 | 0.91 | 0.71 | 0.77 | 0.88 | 0.19 | 6.54 | 1.75 |
| ATM | BRCA1 | RAD51 | pMK2 | 1.88E−04 | 0.80 | 0.91 | 0.71 | 0.77 | 0.88 | 0.19 | 6.54 | 1.75 |
| BRCA1 | RAD51 | XPF | pH2AX | 1.88E−04 | 0.81 | 0.91 | 0.67 | 0.74 | 0.88 | 0.21 | 5.93 | 1.59 |
| BRCA1 | RAD51 | pMK2 | pH2AX | 1.88E−04 | 0.81 | 0.91 | 0.71 | 0.77 | 0.88 | 0.19 | 6.54 | 1.75 |
| ATM | FANCD2 | RAD51 | p53 | 2.05E−04 | 0.87 | 0.95 | 0.67 | 0.75 | 0.93 | 0.19 | 11.25 | 1.67 |
| ERCC1 | FANCD2 | RAD51 | XPF | 2.05E−04 | 0.87 | 0.95 | 0.67 | 0.75 | 0.93 | 0.19 | 11.25 | 1.67 |
| ERCC1 | FANCD2 | RAD51 | p53 | 2.05E−04 | 0.87 | 0.95 | 0.67 | 0.75 | 0.93 | 0.19 | 11.25 | 1.67 |
| FANCD2 | RAD51 | XPF | p53 | 2.05E−04 | 0.88 | 0.95 | 0.67 | 0.75 | 0.93 | 0.19 | 11.25 | 1.67 |
| FANCD2 | RAD51 | p53 | pMK2 | 2.05E−04 | 0.84 | 0.95 | 0.67 | 0.75 | 0.93 | 0.19 | 11.25 | 1.67 |
| BRCA1 | FANCD2 | XPF | pMK2 | 2.45E−04 | 0.84 | 0.87 | 0.73 | 0.77 | 0.84 | 0.20 | 4.87 | 1.69 |
| FANCD2 | XPF | p53 | pH2AX | 2.77E−04 | 0.84 | 0.91 | 0.67 | 0.75 | 0.88 | 0.20 | 6.00 | 1.61 |
| ERCC1 | FANCD2 | XPF | p53 | 2.87E−04 | 0.88 | 0.96 | 0.62 | 0.73 | 0.93 | 0.20 | 10.27 | 1.53 |
| FANCD2 | XPF | p53 | pMK2 | 2.87E−04 | 0.86 | 0.96 | 0.67 | 0.76 | 0.93 | 0.18 | 11.38 | 1.69 |
| FANCD2 | PAR | RAD51 | p53 | 2.99E−04 | 0.90 | 0.95 | 0.71 | 0.76 | 0.94 | 0.17 | 12.16 | 1.80 |
| ATM | BRCA1 | ERCC1 | FANCD2 | 3.59E−04 | 0.84 | 0.91 | 0.62 | 0.72 | 0.87 | 0.23 | 5.43 | 1.47 |
| ERCC1 | RAD51 | XPF | p53 | 4.35E−04 | 0.81 | 0.77 | 0.76 | 0.77 | 0.76 | 0.23 | 3.25 | 1.46 |
| BRCA1 | RAD51 | XPF | p53 | 4.89E−04 | 0.83 | 0.82 | 0.76 | 0.78 | 0.80 | 0.21 | 3.91 | 1.59 |
| ATM | BRCA1 | FANCD2 | XPF | 4.98E−04 | 0.83 | 0.87 | 0.67 | 0.74 | 0.82 | 0.23 | 4.20 | 1.47 |
| BRCA1 | ERCC1 | FANCD2 | XPF | 4.98E−04 | 0.83 | 0.87 | 0.67 | 0.74 | 0.82 | 0.23 | 4.20 | 1.47 |
| BRCA1 | FANCD2 | XPF | p53 | 4.98E−04 | 0.84 | 0.87 | 0.67 | 0.74 | 0.82 | 0.23 | 4.20 | 1.47 |
| BRCA1 | ERCC1 | RAD51 | pMK2 | 5.29E−04 | 0.82 | 0.86 | 0.71 | 0.76 | 0.83 | 0.21 | 4.56 | 1.59 |
| ATM | BRCA1 | FANCD2 | pMK2 | 5.56E−04 | 0.80 | 0.91 | 0.62 | 0.72 | 0.87 | 0.23 | 5.43 | 1.47 |
| BRCA1 | ERCC1 | FANCD2 | pMK2 | 5.56E−04 | 0.81 | 0.91 | 0.67 | 0.75 | 0.88 | 0.20 | 6.00 | 1.61 |
| BRCA1 | FANCD2 | p53 | pMK2 | 5.56E−04 | 0.81 | 0.91 | 0.62 | 0.72 | 0.87 | 0.23 | 5.43 | 1.47 |
| ATM | BRCA1 | RAD51 | pH2AX | 5.62E−04 | 0.83 | 0.91 | 0.62 | 0.71 | 0.87 | 0.23 | 5.36 | 1.44 |
| ATM | RAD51 | XPF | p53 | 6.33E−04 | 0.81 | 0.77 | 0.76 | 0.77 | 0.76 | 0.23 | 3.25 | 1.46 |
| FANCD2 | p53 | pMK2 | pH2AX | 7.05E−04 | 0.80 | 0.96 | 0.57 | 0.71 | 0.92 | 0.23 | 9.23 | 1.38 |
| ATM | FANCD2 | XPF | pH2AX | 7.25E−04 | 0.84 | 0.91 | 0.62 | 0.72 | 0.87 | 0.23 | 5.43 | 1.47 |
| ATM | ERCC1 | FANCD2 | RAD51 | 7.90E−04 | 0.88 | 0.95 | 0.62 | 0.72 | 0.93 | 0.21 | 10.14 | 1.51 |
| ATM | FANCD2 | RAD51 | pH2AX | 7.90E−04 | 0.85 | 0.95 | 0.62 | 0.72 | 0.93 | 0.21 | 10.14 | 1.51 |
| ERCC1 | FANCD2 | RAD51 | pMK2 | 7.90E−04 | 0.85 | 0.95 | 0.62 | 0.72 | 0.93 | 0.21 | 10.14 | 1.51 |
| ERCC1 | FANCD2 | RAD51 | pH2AX | 7.90E−04 | 0.85 | 0.95 | 0.62 | 0.72 | 0.93 | 0.21 | 10.14 | 1.51 |
| FANCD2 | RAD51 | p53 | pH2AX | 7.90E−04 | 0.88 | 0.95 | 0.62 | 0.72 | 0.93 | 0.21 | 10.14 | 1.51 |
| FANCD2 | RAD51 | pMK2 | pH2AX | 7.90E−04 | 0.84 | 0.95 | 0.62 | 0.72 | 0.93 | 0.21 | 10.14 | 1.51 |
| BRCA1 | RAD51 | p53 | pH2AX | 7.99E−04 | 0.84 | 0.91 | 0.62 | 0.71 | 0.87 | 0.23 | 5.36 | 1.44 |
| BRCA1 | ERCC1 | RAD51 | p53 | 8.00E−04 | 0.85 | 0.86 | 0.71 | 0.76 | 0.83 | 0.21 | 4.56 | 1.59 |
| BRCA1 | RAD51 | XPF | pMK2 | 8.00E−04 | 0.81 | 0.86 | 0.71 | 0.76 | 0.83 | 0.21 | 4.56 | 1.59 |
| ATM | ERCC1 | XPF | p53 | 8.52E−04 | 0.78 | 0.61 | 0.86 | 0.82 | 0.67 | 0.27 | 2.47 | 1.39 |
| ATM | ERCC1 | FANCD2 | p53 | 1.04E−03 | 0.89 | 0.96 | 0.57 | 0.71 | 0.92 | 0.23 | 9.23 | 1.38 |
| ATM | ERCC1 | FANCD2 | pMK2 | 1.04E−03 | 0.88 | 0.96 | 0.57 | 0.71 | 0.92 | 0.23 | 9.23 | 1.38 |
| ATM | FANCD2 | p53 | pMK2 | 1.04E−03 | 0.85 | 0.96 | 0.57 | 0.71 | 0.92 | 0.23 | 9.23 | 1.38 |
| ERCC1 | FANCD2 | XPF | pMK2 | 1.04E−03 | 0.87 | 0.96 | 0.62 | 0.73 | 0.93 | 0.20 | 10.27 | 1.53 |
| FANCD2 | XPF | pMK2 | pH2AX | 1.10E−03 | 0.85 | 0.91 | 0.62 | 0.72 | 0.87 | 0.23 | 5.43 | 1.47 |
| FANCD2 | PAR | RAD51 | XPF | 1.13E−03 | 0.88 | 0.95 | 0.62 | 0.70 | 0.93 | 0.22 | 9.85 | 1.47 |
| FANCD2 | PAR | RAD51 | pMK2 | 1.13E−03 | 0.86 | 0.95 | 0.62 | 0.70 | 0.93 | 0.22 | 9.85 | 1.47 |
| BRCA1 | ERCC1 | RAD51 | XPF | 1.21E−03 | 0.80 | 0.77 | 0.76 | 0.77 | 0.76 | 0.23 | 3.25 | 1.46 |
| ERCC1 | XPF | p53 | pMK2 | 1.30E−03 | 0.79 | 0.65 | 0.76 | 0.75 | 0.67 | 0.30 | 2.25 | 1.21 |
| BRCA1 | ERCC1 | FANCD2 | pH2AX | 1.32E−03 | 0.79 | 0.87 | 0.62 | 0.71 | 0.81 | 0.25 | 3.81 | 1.34 |
| BRCA1 | FANCD2 | XPF | pH2AX | 1.32E−03 | 0.82 | 0.87 | 0.62 | 0.71 | 0.81 | 0.25 | 3.81 | 1.34 |
| BRCA1 | FANCD2 | p53 | pH2AX | 1.32E−03 | 0.79 | 0.87 | 0.62 | 0.71 | 0.81 | 0.25 | 3.81 | 1.34 |
| ATM | FANCD2 | p53 | pH2AX | 1.59E−03 | 0.78 | 0.96 | 0.52 | 0.69 | 0.92 | 0.25 | 8.25 | 1.25 |
| ERCC1 | FANCD2 | p53 | pH2AX | 1.59E−03 | 0.80 | 0.96 | 0.52 | 0.69 | 0.92 | 0.25 | 8.25 | 1.25 |
| BRCA1 | FANCD2 | PAR | p53 | 1.90E−03 | 0.84 | 0.90 | 0.57 | 0.68 | 0.86 | 0.26 | 4.75 | 1.28 |
| ATM | BRCA1 | ERCC1 | p53 | 1.97E−03 | 0.81 | 0.87 | 0.62 | 0.71 | 0.81 | 0.25 | 3.81 | 1.34 |
| RAD51 | XPF | p53 | pMK2 | 2.06E−03 | 0.82 | 0.82 | 0.67 | 0.72 | 0.78 | 0.26 | 3.24 | 1.32 |
| BRCA1 | ERCC1 | RAD51 | pH2AX | 2.06E−03 | 0.81 | 0.91 | 0.57 | 0.69 | 0.86 | 0.26 | 4.83 | 1.31 |
| ERCC1 | FANCD2 | p53 | pMK2 | 2.32E−03 | 0.87 | 0.96 | 0.57 | 0.71 | 0.92 | 0.23 | 9.23 | 1.38 |
| BRCA1 | XPF | p53 | pMK2 | 2.40E−03 | 0.80 | 0.83 | 0.67 | 0.73 | 0.78 | 0.25 | 3.29 | 1.34 |
| ERCC1 | FANCD2 | XPF | pH2AX | 2.56E−03 | 0.85 | 0.91 | 0.57 | 0.70 | 0.86 | 0.25 | 4.90 | 1.33 |
| ATM | FANCD2 | RAD51 | pMK2 | 2.56E−03 | 0.84 | 0.95 | 0.57 | 0.70 | 0.92 | 0.23 | 9.10 | 1.36 |
| ATM | FANCD2 | PAR | RAD51 | 2.73E−03 | 0.89 | 0.95 | 0.62 | 0.70 | 0.93 | 0.22 | 9.85 | 1.47 |
| FANCD2 | PAR | RAD51 | pH2AX | 2.81E−03 | 0.89 | 0.95 | 0.57 | 0.68 | 0.92 | 0.24 | 8.82 | 1.32 |
| FANCD2 | RAD51 | XPF | pH2AX | 2.86E−03 | 0.86 | 0.91 | 0.62 | 0.71 | 0.87 | 0.23 | 5.36 | 1.44 |
| ATM | ERCC1 | RAD51 | XPF | 2.89E−03 | 0.80 | 0.77 | 0.71 | 0.74 | 0.75 | 0.26 | 2.96 | 1.33 |
| ATM | RAD51 | XPF | pH2AX | 2.89E−03 | 0.78 | 0.77 | 0.71 | 0.74 | 0.75 | 0.26 | 2.96 | 1.33 |
| FANCD2 | PAR | p53 | pMK2 | 3.03E−03 | 0.85 | 0.95 | 0.62 | 0.71 | 0.93 | 0.21 | 10.00 | 1.49 |
| BRCA1 | ERCC1 | XPF | p53 | 3.30E−03 | 0.79 | 0.74 | 0.71 | 0.74 | 0.71 | 0.27 | 2.59 | 1.26 |
| BRCA1 | ERCC1 | XPF | pMK2 | 4.34E−03 | 0.80 | 0.78 | 0.71 | 0.75 | 0.75 | 0.25 | 3.00 | 1.36 |
| RAD51 | XPF | pMK2 | pH2AX | 4.49E−03 | 0.78 | 0.86 | 0.62 | 0.70 | 0.81 | 0.26 | 3.75 | 1.31 |
| ATM | BRCA1 | FANCD2 | pH2AX | 4.57E−03 | 0.77 | 0.87 | 0.57 | 0.69 | 0.80 | 0.27 | 3.45 | 1.22 |
| BRCA1 | FANCD2 | pMK2 | pH2AX | 4.57E−03 | 0.78 | 0.87 | 0.57 | 0.69 | 0.80 | 0.27 | 3.45 | 1.22 |
| ATM | ERCC1 | FANCD2 | pH2AX | 4.81E−03 | 0.81 | 0.96 | 0.48 | 0.67 | 0.91 | 0.27 | 7.33 | 1.11 |
| ATM | FANCD2 | pMK2 | pH2AX | 4.81E−03 | 0.81 | 0.96 | 0.48 | 0.67 | 0.91 | 0.27 | 7.33 | 1.11 |
| ERCC1 | FANCD2 | pMK2 | pH2AX | 4.81E−03 | 0.81 | 0.96 | 0.48 | 0.67 | 0.91 | 0.27 | 7.33 | 1.11 |
| FANCD2 | PAR | XPF | p53 | 5.44E−03 | 0.87 | 0.90 | 0.57 | 0.68 | 0.86 | 0.26 | 4.75 | 1.28 |
| ATM | BRCA1 | ERCC1 | pH2AX | 5.89E−03 | 0.74 | 0.91 | 0.48 | 0.66 | 0.83 | 0.30 | 3.94 | 1.08 |
| ATM | BRCA1 | p53 | pH2AX | 5.89E−03 | 0.75 | 0.91 | 0.48 | 0.66 | 0.83 | 0.30 | 3.94 | 1.08 |
| ATM | BRCA1 | pMK2 | pH2AX | 5.89E−03 | 0.72 | 0.91 | 0.52 | 0.68 | 0.85 | 0.27 | 4.40 | 1.20 |
| BRCA1 | ERCC1 | pMK2 | pH2AX | 5.89E−03 | 0.72 | 0.91 | 0.52 | 0.68 | 0.85 | 0.27 | 4.40 | 1.20 |
| BRCA1 | p53 | pMK2 | pH2AX | 5.89E−03 | 0.73 | 0.91 | 0.52 | 0.68 | 0.85 | 0.27 | 4.40 | 1.20 |
| ATM | BRCA1 | FANCD2 | PAR | 5.92E−03 | 0.85 | 0.90 | 0.52 | 0.66 | 0.85 | 0.29 | 4.26 | 1.16 |
| BRCA1 | ERCC1 | FANCD2 | PAR | 5.92E−03 | 0.84 | 0.90 | 0.57 | 0.68 | 0.86 | 0.26 | 4.75 | 1.28 |
| BRCA1 | FANCD2 | PAR | pMK2 | 5.92E−03 | 0.83 | 0.90 | 0.52 | 0.66 | 0.85 | 0.29 | 4.26 | 1.16 |
| BRCA1 | FANCD2 | PAR | XPF | 6.45E−03 | 0.85 | 0.86 | 0.62 | 0.69 | 0.81 | 0.26 | 3.69 | 1.29 |
| ERCC1 | FANCD2 | PAR | RAD51 | 6.60E−03 | 0.88 | 0.90 | 0.62 | 0.69 | 0.87 | 0.24 | 5.19 | 1.39 |
| ERCC1 | FANCD2 | PAR | XPF | 6.60E−03 | 0.87 | 0.95 | 0.52 | 0.67 | 0.92 | 0.26 | 8.00 | 1.20 |
| ATM | BRCA1 | XPF | p53 | 6.88E−03 | 0.77 | 0.70 | 0.76 | 0.76 | 0.70 | 0.27 | 2.50 | 1.29 |
| BRCA1 | PAR | RAD51 | pH2AX | 7.15E−03 | 0.85 | 0.90 | 0.57 | 0.67 | 0.86 | 0.27 | 4.67 | 1.26 |
| ATM | BRCA1 | RAD51 | XPF | 7.76E−03 | 0.81 | 0.77 | 0.71 | 0.74 | 0.75 | 0.26 | 2.96 | 1.33 |
| ATM | RAD51 | XPF | pMK2 | 8.05E−03 | 0.79 | 0.82 | 0.67 | 0.72 | 0.78 | 0.26 | 3.24 | 1.32 |
| BRCA1 | PAR | RAD51 | p53 | 8.44E−03 | 0.89 | 0.90 | 0.62 | 0.69 | 0.87 | 0.24 | 5.19 | 1.39 |
| ATM | FANCD2 | PAR | XPF | 8.56E−03 | 0.86 | 0.95 | 0.52 | 0.67 | 0.92 | 0.26 | 8.00 | 1.20 |
| BRCA1 | PAR | RAD51 | XPF | 8.68E−03 | 0.88 | 0.90 | 0.57 | 0.67 | 0.86 | 0.27 | 4.67 | 1.26 |
| BRCA1 | ERCC1 | p53 | pMK2 | 8.92E−03 | 0.77 | 0.83 | 0.67 | 0.73 | 0.78 | 0.25 | 3.29 | 1.34 |
| ATM | BRCA1 | p53 | pMK2 | 9.81E−03 | 0.78 | 0.83 | 0.62 | 0.70 | 0.76 | 0.27 | 2.99 | 1.23 |
| ATM | BRCA1 | ERCC1 | XPF | 1.06E−02 | 0.78 | 0.74 | 0.67 | 0.71 | 0.70 | 0.30 | 2.36 | 1.15 |
| PAR | RAD51 | XPF | p53 | 1.12E−02 | 0.88 | 0.90 | 0.57 | 0.67 | 0.86 | 0.27 | 4.67 | 1.26 |
| ATM | FANCD2 | PAR | p53 | 1.13E−02 | 0.87 | 0.90 | 0.57 | 0.68 | 0.86 | 0.26 | 4.75 | 1.28 |
| ATM | BRCA1 | PAR | XPF | 1.26E−02 | 0.78 | 0.86 | 0.52 | 0.64 | 0.79 | 0.31 | 3.00 | 1.06 |
| BRCA1 | PAR | XPF | p53 | 1.26E−02 | 0.81 | 0.86 | 0.52 | 0.64 | 0.79 | 0.31 | 3.00 | 1.06 |
| BRCA1 | PAR | XPF | pMK2 | 1.26E−02 | 0.78 | 0.86 | 0.52 | 0.64 | 0.79 | 0.31 | 3.00 | 1.06 |
| ERCC1 | RAD51 | XPF | pH2AX | 1.32E−02 | 0.77 | 0.86 | 0.57 | 0.68 | 0.80 | 0.28 | 3.39 | 1.19 |
| RAD51 | XPF | p53 | pH2AX | 1.32E−02 | 0.80 | 0.86 | 0.57 | 0.68 | 0.80 | 0.28 | 3.39 | 1.19 |
| BRCA1 | ERCC1 | XPF | pH2AX | 1.40E−02 | 0.76 | 0.83 | 0.57 | 0.68 | 0.75 | 0.30 | 2.71 | 1.12 |
| BRCA1 | XPF | p53 | pH2AX | 1.40E−02 | 0.77 | 0.83 | 0.52 | 0.66 | 0.73 | 0.32 | 2.46 | 1.02 |
| ERCC1 | RAD51 | p53 | pH2AX | 1.47E−02 | 0.74 | 0.91 | 0.48 | 0.65 | 0.83 | 0.30 | 3.87 | 1.06 |
| ERCC1 | FANCD2 | PAR | p53 | 1.52E−02 | 0.87 | 0.90 | 0.57 | 0.68 | 0.86 | 0.26 | 4.75 | 1.28 |
| ATM | ERCC1 | RAD51 | pMK2 | 1.63E−02 | 0.78 | 0.86 | 0.62 | 0.70 | 0.81 | 0.26 | 3.75 | 1.31 |
| ATM | FANCD2 | PAR | pMK2 | 1.65E−02 | 0.85 | 0.95 | 0.52 | 0.67 | 0.92 | 0.26 | 8.00 | 1.20 |
| ERCC1 | FANCD2 | PAR | pH2AX | 1.65E−02 | 0.83 | 0.95 | 0.52 | 0.67 | 0.92 | 0.26 | 8.00 | 1.20 |
| FANCD2 | PAR | XPF | pMK2 | 1.69E−02 | 0.85 | 0.95 | 0.48 | 0.65 | 0.91 | 0.29 | 7.10 | 1.08 |
| FANCD2 | PAR | p53 | pH2AX | 1.69E−02 | 0.84 | 0.95 | 0.43 | 0.63 | 0.90 | 0.31 | 6.25 | 0.95 |
| FANCD2 | PAR | pMK2 | pH2AX | 1.69E−02 | 0.81 | 0.95 | 0.43 | 0.63 | 0.90 | 0.31 | 6.25 | 0.95 |
| ATM | BRCA1 | PAR | RAD51 | 1.79E−02 | 0.88 | 0.85 | 0.62 | 0.68 | 0.81 | 0.27 | 3.63 | 1.26 |
| BRCA1 | FANCD2 | PAR | pH2AX | 1.80E−02 | 0.81 | 0.86 | 0.52 | 0.64 | 0.79 | 0.31 | 3.00 | 1.06 |
| ATM | BRCA1 | ERCC1 | pMK2 | 1.82E−02 | 0.77 | 0.87 | 0.57 | 0.69 | 0.80 | 0.27 | 3.45 | 1.22 |
| BRCA1 | ERCC1 | p53 | pH2AX | 1.82E−02 | 0.75 | 0.87 | 0.48 | 0.65 | 0.77 | 0.32 | 2.80 | 1.00 |
| ATM | PAR | XPF | p53 | 1.99E−02 | 0.80 | 0.81 | 0.52 | 0.63 | 0.73 | 0.33 | 2.36 | 0.97 |
| BRCA1 | PAR | p53 | pH2AX | 2.02E−02 | 0.78 | 0.90 | 0.52 | 0.66 | 0.85 | 0.29 | 4.26 | 1.16 |
| BRCA1 | ERCC1 | PAR | p53 | 2.06E−02 | 0.78 | 0.86 | 0.52 | 0.64 | 0.79 | 0.31 | 3.00 | 1.06 |
| ATM | BRCA1 | PAR | pH2AX | 2.07E−02 | 0.75 | 0.90 | 0.48 | 0.63 | 0.83 | 0.31 | 3.80 | 1.04 |
| BRCA1 | PAR | XPF | pH2AX | 2.07E−02 | 0.76 | 0.90 | 0.48 | 0.63 | 0.83 | 0.31 | 3.80 | 1.04 |
| BRCA1 | PAR | pMK2 | pH2AX | 2.07E−02 | 0.73 | 0.90 | 0.48 | 0.63 | 0.83 | 0.31 | 3.80 | 1.04 |
| ATM | PAR | RAD51 | p53 | 2.11E−02 | 0.89 | 0.95 | 0.52 | 0.66 | 0.92 | 0.27 | 7.86 | 1.18 |
| ATM | BRCA1 | XPF | pMK2 | 2.21E−02 | 0.79 | 0.78 | 0.67 | 0.72 | 0.74 | 0.27 | 2.74 | 1.24 |
| ERCC1 | XPF | pMK2 | pH2AX | 2.22E−02 | 0.73 | 0.83 | 0.52 | 0.66 | 0.73 | 0.32 | 2.46 | 1.02 |
| ERCC1 | XPF | p53 | pH2AX | 2.22E−02 | 0.73 | 0.78 | 0.57 | 0.67 | 0.71 | 0.32 | 2.27 | 1.04 |
| ERCC1 | PAR | RAD51 | XPF | 2.89E−02 | 0.81 | 0.90 | 0.48 | 0.62 | 0.83 | 0.32 | 3.72 | 1.02 |
| ERCC1 | PAR | XPF | p53 | 3.19E−02 | 0.78 | 0.86 | 0.48 | 0.62 | 0.77 | 0.33 | 2.69 | 0.96 |
| ATM | ERCC1 | XPF | pH2AX | 3.22E−02 | 0.73 | 0.78 | 0.57 | 0.67 | 0.71 | 0.32 | 2.27 | 1.04 |
| ATM | XPF | pMK2 | pH2AX | 3.22E−02 | 0.75 | 0.78 | 0.62 | 0.69 | 0.72 | 0.30 | 2.49 | 1.13 |
| ATM | RAD51 | p53 | pMK2 | 3.55E−02 | 0.81 | 0.82 | 0.62 | 0.69 | 0.76 | 0.28 | 2.94 | 1.20 |
| ATM | RAD51 | p53 | pH2AX | 3.59E−02 | 0.80 | 0.91 | 0.48 | 0.65 | 0.83 | 0.30 | 3.87 | 1.06 |
| BRCA1 | XPF | pMK2 | pH2AX | 3.65E−02 | 0.77 | 0.83 | 0.52 | 0.66 | 0.73 | 0.32 | 2.46 | 1.02 |
| ATM | ERCC1 | FANCD2 | PAR | 3.67E−02 | 0.88 | 0.90 | 0.52 | 0.66 | 0.85 | 0.29 | 4.26 | 1.16 |
| ERCC1 | FANCD2 | PAR | pMK2 | 3.67E−02 | 0.83 | 0.90 | 0.52 | 0.66 | 0.85 | 0.29 | 4.26 | 1.16 |
| BRCA1 | PAR | RAD51 | pMK2 | 3.75E−02 | 0.85 | 0.85 | 0.57 | 0.65 | 0.80 | 0.29 | 3.27 | 1.14 |
| XPF | p53 | pMK2 | pH2AX | 3.91E−02 | 0.74 | 0.78 | 0.52 | 0.64 | 0.69 | 0.34 | 2.06 | 0.95 |
| ATM | FANCD2 | PAR | pH2AX | 3.92E−02 | 0.84 | 0.95 | 0.38 | 0.61 | 0.89 | 0.33 | 5.45 | 0.84 |
| ATM | BRCA1 | XPF | pH2AX | 3.93E−02 | 0.76 | 0.83 | 0.43 | 0.61 | 0.69 | 0.36 | 1.99 | 0.84 |
| ATM | RAD51 | pMK2 | pH2AX | 4.07E−02 | 0.77 | 0.86 | 0.57 | 0.68 | 0.80 | 0.28 | 3.39 | 1.19 |
| ATM | PAR | RAD51 | pMK2 | 4.09E−02 | 0.82 | 0.95 | 0.48 | 0.63 | 0.91 | 0.29 | 6.97 | 1.05 |
| ATM | XPF | p53 | pH2AX | 4.14E−02 | 0.73 | 0.74 | 0.57 | 0.65 | 0.67 | 0.34 | 1.96 | 0.96 |
| ERCC1 | RAD51 | XPF | pMK2 | 4.37E−02 | 0.78 | 0.73 | 0.62 | 0.67 | 0.68 | 0.33 | 2.11 | 1.03 |
| ERCC1 | RAD51 | p53 | pMK2 | 4.55E−02 | 0.79 | 0.82 | 0.57 | 0.67 | 0.75 | 0.30 | 2.67 | 1.10 |
| BRCA1 | ERCC1 | PAR | RAD51 | 4.56E−02 | 0.85 | 0.85 | 0.52 | 0.63 | 0.79 | 0.32 | 2.94 | 1.03 |
| ATM | ERCC1 | p53 | pMK2 | 4.82E−02 | 0.79 | 0.83 | 0.57 | 0.68 | 0.75 | 0.30 | 2.71 | 1.12 |
| ATM | ERCC1 | PAR | RAD51 | 4.90E−02 | 0.83 | 0.95 | 0.43 | 0.61 | 0.90 | 0.32 | 6.13 | 0.93 |
| ATM | BRCA1 | ERCC1 | PAR | 4.95E−02 | 0.78 | 0.86 | 0.48 | 0.62 | 0.77 | 0.33 | 2.69 | 0.96 |
| BRCA1 | ERCC1 | PAR | XPF | 5.07E−02 | 0.77 | 0.86 | 0.43 | 0.60 | 0.75 | 0.36 | 2.40 | 0.86 |
| PAR | RAD51 | XPF | pH2AX | 5.53E−02 | 0.81 | 0.95 | 0.43 | 0.61 | 0.90 | 0.32 | 6.13 | 0.93 |
| ATM | BRCA1 | PAR | p53 | 5.94E−02 | 0.81 | 0.81 | 0.52 | 0.63 | 0.73 | 0.33 | 2.36 | 0.97 |
| ATM | BRCA1 | PAR | pMK2 | 5.94E−02 | 0.77 | 0.81 | 0.52 | 0.63 | 0.73 | 0.33 | 2.36 | 0.97 |
| ERCC1 | PAR | RAD51 | p53 | 6.06E−02 | 0.84 | 0.90 | 0.52 | 0.64 | 0.85 | 0.29 | 4.18 | 1.13 |
| PAR | RAD51 | p53 | pMK2 | 6.06E−02 | 0.84 | 0.90 | 0.52 | 0.64 | 0.85 | 0.29 | 4.18 | 1.13 |
| PAR | RAD51 | XPF | pMK2 | 6.20E−02 | 0.83 | 0.90 | 0.48 | 0.62 | 0.83 | 0.32 | 3.72 | 1.02 |
| ATM | ERCC1 | RAD51 | pH2AX | 6.63E−02 | 0.78 | 0.91 | 0.43 | 0.63 | 0.82 | 0.33 | 3.44 | 0.95 |
| PAR | XPF | p53 | pH2AX | 8.20E−02 | 0.76 | 0.90 | 0.38 | 0.59 | 0.80 | 0.36 | 2.97 | 0.83 |
| ATM | ERCC1 | RAD51 | p53 | 8.58E−02 | 0.83 | 0.82 | 0.52 | 0.64 | 0.73 | 0.33 | 2.41 | 1.00 |
| ERCC1 | PAR | XPF | pMK2 | 8.65E−02 | 0.74 | 0.86 | 0.38 | 0.58 | 0.73 | 0.38 | 2.13 | 0.78 |
| ERCC1 | RAD51 | pMK2 | pH2AX | 9.06E−02 | 0.74 | 0.86 | 0.43 | 0.61 | 0.75 | 0.35 | 2.45 | 0.88 |
| RAD51 | p53 | pMK2 | pH2AX | 9.06E−02 | 0.80 | 0.86 | 0.43 | 0.61 | 0.75 | 0.35 | 2.45 | 0.88 |
| BRCA1 | ERCC1 | PAR | pMK2 | 1.07E−01 | 0.74 | 0.86 | 0.43 | 0.60 | 0.75 | 0.36 | 2.40 | 0.86 |
| ATM | PAR | XPF | pMK2 | 1.09E−01 | 0.78 | 0.81 | 0.43 | 0.59 | 0.69 | 0.38 | 1.91 | 0.80 |
| ATM | PAR | RAD51 | pH2AX | 1.11E−01 | 0.81 | 0.90 | 0.48 | 0.62 | 0.83 | 0.32 | 3.72 | 1.02 |
| PAR | RAD51 | p53 | pH2AX | 1.11E−01 | 0.85 | 0.90 | 0.48 | 0.62 | 0.83 | 0.32 | 3.72 | 1.02 |
| PAR | RAD51 | pMK2 | pH2AX | 1.11E−01 | 0.80 | 0.90 | 0.48 | 0.62 | 0.83 | 0.32 | 3.72 | 1.02 |
| BRCA1 | ERCC1 | PAR | pH2AX | 1.11E−01 | 0.73 | 0.81 | 0.52 | 0.63 | 0.73 | 0.33 | 2.36 | 0.97 |
| ATM | PAR | RAD51 | XPF | 1.13E−01 | 0.84 | 0.90 | 0.48 | 0.62 | 0.83 | 0.32 | 3.72 | 1.02 |
| BRCA1 | PAR | p53 | pMK2 | 1.27E−01 | 0.77 | 0.81 | 0.48 | 0.61 | 0.71 | 0.36 | 2.13 | 0.88 |
| FANCD2 | PAR | XPF | pH2AX | 1.47E−01 | 0.78 | 0.86 | 0.38 | 0.59 | 0.73 | 0.37 | 2.18 | 0.80 |
| ATM | ERCC1 | p53 | pH2AX | 1.48E−01 | 0.69 | 0.91 | 0.29 | 0.58 | 0.75 | 0.39 | 2.33 | 0.68 |
| PAR | XPF | p53 | pMK2 | 1.51E−01 | 0.81 | 0.86 | 0.38 | 0.58 | 0.73 | 0.38 | 2.13 | 0.78 |
| ATM | ERCC1 | pMK2 | pH2AX | 1.63E−01 | 0.68 | 0.87 | 0.43 | 0.63 | 0.75 | 0.34 | 2.50 | 0.91 |
| ATM | PAR | XPF | pH2AX | 1.68E−01 | 0.75 | 0.90 | 0.33 | 0.58 | 0.78 | 0.38 | 2.59 | 0.74 |
| PAR | XPF | pMK2 | pH2AX | 1.68E−01 | 0.73 | 0.90 | 0.33 | 0.58 | 0.78 | 0.38 | 2.59 | 0.74 |
| ATM | ERCC1 | PAR | XPF | 2.49E−01 | 0.76 | 0.81 | 0.33 | 0.55 | 0.64 | 0.43 | 1.51 | 0.65 |
| ATM | PAR | p53 | pMK2 | 2.54E−01 | 0.77 | 0.90 | 0.33 | 0.58 | 0.78 | 0.38 | 2.59 | 0.74 |
| ERCC1 | PAR | XPF | pH2AX | 2.55E−01 | 0.72 | 0.86 | 0.38 | 0.58 | 0.73 | 0.38 | 2.13 | 0.78 |
| ERCC1 | PAR | RAD51 | pH2AX | 2.74E−01 | 0.79 | 0.85 | 0.48 | 0.61 | 0.77 | 0.34 | 2.63 | 0.93 |
| ATM | PAR | p53 | pH2AX | 2.83E−01 | 0.78 | 0.86 | 0.38 | 0.58 | 0.73 | 0.38 | 2.13 | 0.78 |
| ATM | PAR | pMK2 | pH2AX | 2.83E−01 | 0.69 | 0.86 | 0.38 | 0.58 | 0.73 | 0.38 | 2.13 | 0.78 |
| PAR | p53 | pMK2 | pH2AX | 2.83E−01 | 0.72 | 0.86 | 0.38 | 0.58 | 0.73 | 0.38 | 2.13 | 0.78 |
| ATM | ERCC1 | PAR | p53 | 2.94E−01 | 0.80 | 0.90 | 0.29 | 0.56 | 0.75 | 0.40 | 2.24 | 0.65 |
| ATM | ERCC1 | PAR | pMK2 | 2.94E−01 | 0.72 | 0.90 | 0.29 | 0.56 | 0.75 | 0.40 | 2.24 | 0.65 |
| ERCC1 | PAR | RAD51 | pMK2 | 3.18E−01 | 0.77 | 0.85 | 0.43 | 0.59 | 0.75 | 0.37 | 2.34 | 0.84 |
| ATM | ERCC1 | PAR | pH2AX | 4.51E−01 | 0.71 | 0.86 | 0.38 | 0.58 | 0.73 | 0.38 | 2.13 | 0.78 |
| ERCC1 | PAR | pMK2 | pH2AX | 4.51E−01 | 0.63 | 0.86 | 0.38 | 0.58 | 0.73 | 0.38 | 2.13 | 0.78 |
| ERCC1 | PAR | p53 | pH2AX | 5.64E−01 | 0.68 | 0.81 | 0.38 | 0.57 | 0.67 | 0.40 | 1.70 | 0.72 |
| ATM | p53 | pMK2 | pH2AX | 5.66E−01 | 0.67 | 0.87 | 0.29 | 0.57 | 0.67 | 0.41 | 1.71 | 0.65 |
| ERCC1 | p53 | pMK2 | pH2AX | 7.70E−01 | 0.64 | 0.83 | 0.33 | 0.58 | 0.64 | 0.41 | 1.58 | 0.69 |
| ERCC1 | PAR | p53 | pMK2 | 9.89E−01 | 0.71 | 0.81 | 0.29 | 0.53 | 0.60 | 0.45 | 1.33 | 0.58 |

TABLE 10
HNCMARKERS and response to Induction
Chemotherapy in Head and Neck Cancer

| | | | % Correct |
|---|---|---|---|
| | | | Responders |
| | DNA | | at 100% |
| | Repair | ROC plot/ | SD/PD |
| Marker | Pathway | AUC value | Correct |
| XPF | NER | 0.783 | 60 |
| pMK2 | DDR | 0.707 | 47 |
| MLH 1 | MMR | 0.545 | 23 |
| PARP1 | BER | 0.571 | 20 |
| PAR | BER | 0.509 | 17 |
| FANCD2 | FA/HR | 0.571 | 13 |
| ERCC1 | NER | 0.569 | 7 |
| pH2AX | DDR/NHEJ | 0.519 | — |
| Ki67 | Cell | 0.702 | — |
| | proliferation | | |

### REFERENCE

Parkin D M, Bray F, Ferlay J, Pisani P et al. (2001) Estimating the world cancer burden: Globocan 2000. Int J Cancer 94: 153-156
Seiwert T Y, Salama J K, Vokes E E. The chemoradiation paradigm in head and neck cancer. Nat Clin Pract Oncol. 2007 4(3):156-71
Salama J K, Seiwert T Y, Vokes E E. Chemoradiotherapy for locally advanced head and neck cancer. J Clin Oncol. 2007 Sep. 10; 25(26):4118-26
Salama J K, Stenson K M, Kistner E O, Mittal B B, Argiris A, Witt M E, Rosen F, Brockstein B E, Cohen E E, Haraf D J, Vokes E E. Induction chemotherapy and concurrent chemoradiotherapy for locoregionally advanced head and neck cancer: a multi-institutional phase II trial investigating three radiotherapy dose levels. Ann Oncol. 2008 October; 19(10):1787-94.
Vokes, E. E., Kies, M. S., Haraf, D J., Stenson, K., List, M., Humerickhouse, R., Dolan, M. E., Pelzer, H., Sulzen, L., Witt, M. E., Hsieh, Y. C., Mittal, B. B., and Weichselbaum, R. R. Concomitant chemoradiotherapy as primary therapy for locoregionally advanced head and neck cancer. J. Clin. Oncol., 18: 1652-1661, 2000.
Kies, M. S., Haraf, D. J., Rosen, F., Stenson, K., List, M., Brockstein, B., Chung, T., Mittal, B. B., Pelzer, H., Portugal, L., Rademaker, A., Weichselbaum, R., and Vokes, E. E. Concomitant infusional paclitaxel and fluorouracil, oral hydroxyurea, and hyperfractionated radiation for locally advanced squamous head and neck cancer. J. Clin. Oncol., 19: 1961-1969, 2001.
Michiels S., Le Maître A., Buyse M., Burzykowski T., Maillard E., Bogaerts J., Vermorken J., Budach W., Pajak T., Ang K. Surrogate endpoints for overall survival in locally advanced head and neck cancer: meta-analyses of individual patient data The Lancet Oncology, 10, 341-350, 2009.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of accessing the effectiveness of a treatment regimen treatment of a subject having a head and neck cancer comprising
a) detecting the level of an effective amount of one or more HNCMARKERS in a sample from the subject, and
b) comparing the level of the effective amount of the one or more HNCMARKERS to a reference value.

2. A method of monitoring a treatment regimen of a subject with head and neck cancer comprising
a) detecting the level of an effective amount of one or more HNCMARKERS in a first sample from the subject at a first period of time;
b) detecting the level of an effective amount of one or more HNCMARKERS in a second sample from the subject at a second period of time;
c) comparing the level of the effective amount of one or more HNCMARKERS detected in step (a) to the amount detected in step (b), or to a reference value.

3. A method of determining whether a subject with head and neck cancer would derive a benefit from a treatment regimen
a) detecting the level of an effective amount of one or more HNCMARKERS and
b) comparing the level of the effective amount of one or more HNCMARKERS detected in step (a) to a reference value.

4. A method for predicting the survivability of a head and neck cancer-diagnosed subject comprising
a) detecting the level of an effective amount of one or more HNCMARKERS in a sample from the subject, and
b) comparing the level of the effective amount of the one or more HNCMARKERS to a reference value.

5. The method of claim 4, wherein said subject has received treated for head and neck cancer

6. The method of claim 5, wherein said treatment is immunotherapy, induction chemotherapy, concurrent chemoradiotherapy or a combination thereof.

7. The method of claim 4, wherein said HNCMARKER is selected from the group consisting of XPF, FANCD2, RAD51, BRCA1, ATM, PAR, p53, ERCC1, pH2AX, and pMK2.

8. The method of claim 7, comprising detecting
a) XPF and at least one HNCMARKER selected from the group consisting of FANCD2, pMAPKAP Kinase 2 (pMK2), pH2AX, BRCA1, PAR, ATM, ERCC1, RAD51, p53, POL H, MUS81, p16, and HPV
b) FANCD2 and at least one HNCMARKER selected from the group consisting of XPF, pMAPKAP Kinase 2 (pMK2), pH2AX, BRCA1, PAR, ATM, ERCC1, RAD51, p53, POL H, MUS81, p16, and HPV.
c) pMAPKAP Kinase 2 (pMK2) and at least one HNCMARKER selected from the group consisting of XPF, FANCD2, pH2AX, BRCA1, PAR, ATM, ERCC1, RAD51, p53, POL H, MUS81, p16, and HPV.
d) pH2AX and at least one HNCMARKER selected from the group consisting of XPF, FANCD2, pMAPKAP Kinase 2 (pMK2), BRCA1, PAR, ATM, ERCC1, RAD51, p53, POL H, MUS81, p16, and HPV.
e) BRCA1 and at least one HNCMARKER selected from the group consisting of XPF, FANCD2, pMAPKAP Kinase 2 (pMK2), pH2AX, PAR, ATM, ERCC1, RAD51, p53, POL H, MUS81, p16, and HPV.
f) PAR and at least one HNCMARKER selected from the group consisting of XPF, FANCD2, pMAPKAP Kinase 2 (pMK2), pH2AX, BRCA1, ATM, ERCC1, RAD51, p53, POL H, MUS81, p16, and HPV.
g) ATM and at least one HNCMARKER selected from the group consisting of XPF, FANCD2, pMAPKAP Kinase 2 (pMK2), pH2AX, BRCA1, PAR, ERCC1, RAD51, p53, POL H, MUS81, p16, and HPV.
h) ERCC1 and at least one HNCMARKER selected from the group consisting of XPF, FANCD2, pMAPKAP Kinase 2 (pMK2), pH2AX, BRCA1, PAR, ATM, RAD51, p53, POL H, MUS81, p16, and HPV.
i) RAD51 and at least one HNCMARKER selected from the group consisting of XPF, FANCD2, pMAPKAP Kinase 2 (pMK2), pH2AX, BRCA1, PAR, ATM, ERCC1, p53, POL H, MUS81, p16, and HPV.
j) p53, and at least one HNCMARKER selected from the group consisting of XPF, FANCD2, pMAPKAP Kinase 2 (pMK2), pH2AX, BRCA1, PAR, ATM, ERCC1, RAD51, POL H, MUS81, p16, and HPV.
k) POL H and at least one HNCMARKER selected from the group consisting of XPF, FANCD2, pMAPKAP Kinase 2 (pMK2), pH2AX, BRCA1, PAR, ATM, ERCC1, RAD51, p53, MUS81, p16, and HPV.
l) MUS81 and at least one HNCMARKER selected from the group consisting of XPF, FANCD2, pMAPKAP Kinase 2 (pMK2), pH2AX, BRCA1, PAR, ATM, ERCC1, RAD51, p53, POL H, p16, and HPV.
m) p16 and at least one HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and HPV.
n) HPV and at least one HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and p16.

9. The method of claim 4, comprising measuring
a) XPF and at least two HNCMARKER selected from the group consisting of FANCD2, pMAPKAP Kinase 2 (pMK2), pH2AX, BRCA1, PAR, ATM, ERCC1, RAD51, p53, POL H, MUS81, p16, and HPV
b) FANCD2 and at least two HNCMARKER selected from the group consisting of XPF, pMAPKAP Kinase 2 (pMK2), pH2AX, BRCA1, PAR, ATM, ERCC1, RAD51, p53, POL H, MUS81, p16, and HPV.
c) pMAPKAP Kinase 2 (pMK2) and at least two HNCMARKER selected from the group consisting of XPF, FANCD2, pH2AX, BRCA1, PAR, ATM, ERCC1, RAD51, p53, POL H, MUS81, p16, and HPV.
d) pH2AX and at least two HNCMARKER selected from the group consisting of XPF, FANCD2, pMAPKAP Kinase 2 (pMK2), BRCA1, PAR, ATM, ERCC1, RAD51, p53, POL H, MUS81, p16, and HPV.
e) BRCA1 and at least two HNCMARKER selected from the group consisting of XPF, FANCD2, pMAPKAP Kinase 2 (pMK2), pH2AX, PAR, ATM, ERCC1, RAD51, p53, POL H, MUS81, p16, and HPV.
f) PAR and at least two HNCMARKER selected from the group consisting of XPF, FANCD2, pMAPKAP Kinase 2 (pMK2), pH2AX, BRCA1, ATM, ERCC1, RAD51, p53, POL H, MUS81, p16, and HPV.
g) ATM and at least two HNCMARKER selected from the group consisting of XPF, FANCD2, pMAPKAP Kinase 2 (pMK2), pH2AX, BRCA1, PAR, ERCC1, RAD51, p53, POL H, MUS81, p16, and HPV.
h) ERCC1 and at least two HNCMARKER selected from the group consisting of XPF, FANCD2, pMAPKAP Kinase 2 (pMK2), pH2AX, BRCA1, PAR, ATM, RAD51, p53, POL H, MUS81, p16, and HPV.
i) RAD51 and at least two HNCMARKER selected from the group consisting of XPF, FANCD2, pMAPKAP Kinase 2 (pMK2), pH2AX, BRCA1, PAR, ATM, ERCC1, p53, POL H, MUS81, p16, and HPV.
j) p53, and at least two HNCMARKER selected from the group consisting of XPF, FANCD2, pMAPKAP Kinase 2 (pMK2), pH2AX, BRCA1, PAR, ATM, ERCC1, RAD51, POL H, MUS81, p16, and HPV.
k) POL H and at least two HNCMARKER selected from the group consisting of XPF, FANCD2, pMAPKAP Kinase 2 (pMK2), pH2AX, BRCA1, PAR, ATM, ERCC1, RAD51, p53, MUS81, p16, and HPV.
l) MUS81 and at least two HNCMARKER selected from the group consisting of XPF, FANCD2, pMAPKAP Kinase 2 (pMK2), pH2AX, BRCA1, PAR, ATM, ERCC1, RAD51, p53, POL H, p16, and HPV.
m) p16 and at least two HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and HPV.
n) HPV and at least two HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and p16.

10. The method of claim 4, comprising measuring
a) XPF and at least three HNCMARKER selected from the group consisting of FANCD2, pMAPKAP Kinase 2 (pMK2), pH2AX, BRCA1, PAR, ATM, ERCC1, RAD51, p53, POL H, MUS81, p16, and HPV
b) FANCD2 and at least three HNCMARKER selected from the group consisting of XPF, pMAPKAP Kinase 2 (pMK2), pH2AX, BRCA1, PAR, ATM, ERCC1, RAD51, p53, POL H, MUS81, p16, and HPV.
c) pMAPKAP Kinase 2 (pMK2) and at least three HNCMARKER selected from the group consisting of XPF, FANCD2, pH2AX, BRCA1, PAR, ATM, ERCC1, RAD51, p53, POL H, MUS81, p16, and HPV.
d) pH2AX and at least three HNCMARKER selected from the group consisting of XPF, FANCD2, pMAPKAP Kinase 2 (pMK2), BRCA1, PAR, ATM, ERCC1, RAD51, p53, POL H, MUS81, p16, and HPV.
e) BRCA1 and at least three HNCMARKER selected from the group consisting of XPF, FANCD2, pMAPKAP Kinase 2 (pMK2), pH2AX, PAR, ATM, ERCC1, RAD51, p53, POL H, MUS81, p16, and HPV.
f) PAR and at least three HNCMARKER selected from the group consisting of XPF, FANCD2, pMAPKAP Kinase 2 (pMK2), pH2AX, BRCA1, ATM, ERCC1, RAD51, p53, POL H, MUS81, p16, and HPV.
g) ATM and at least three HNCMARKER selected from the group consisting of XPF, FANCD2, pMAPKAP Kinase 2 (pMK2), pH2AX, BRCA1, PAR, ERCC1, RAD51, p53, POL H, MUS81, p16, and HPV.
h) ERCC1 and at least three HNCMARKER selected from the group consisting of XPF, FANCD2, pMAPKAP Kinase 2 (pMK2), pH2AX, BRCA1, PAR, ATM, RAD51, p53, POL H, MUS81, p16, and HPV.
i) RAD51 and at least three HNCMARKER selected from the group consisting of XPF, FANCD2, pMAPKAP Kinase 2 (pMK2), pH2AX, BRCA1, PAR, ATM, ERCC1, p53, POL H, MUS81, p16, and HPV.
j) p53, and at least three HNCMARKER selected from the group consisting of XPF, FANCD2, pMAPKAP Kinase 2 (pMK2), pH2AX, BRCA1, PAR, ATM, ERCC1, RAD51, POL H, MUS81, p16, and HPV.
k) POL H and at least three HNCMARKER selected from the group consisting of XPF, FANCD2, pMAPKAP Kinase 2 (pMK2), pH2AX, BRCA1, PAR, ATM, ERCC1, RAD51, p53, MUS81, p16, and HPV.
l) MUS81 and at least three HNCMARKER selected from the group consisting of XPF, FANCD2, pMAPKAP Kinase 2 (pMK2), pH2AX, BRCA1, PAR, ATM, ERCC1, RAD51, p53, POL H, p16, and HPV.
m) p16 and at least three HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and HPV.
n) HPV and at least three HNCMARKER selected from the group consisting of XPF, FANCD2, pMAPKAP Kinase 2 (pMK2), pH2AX, BRCA1, PAR, ATM, ERCC1, RAD51, p53, POL H, MUS81, and p16.

11. A method of determining the sensitivity of a head and neck cancer to a chemotherapeutic agent comprising identifying an alteration in at least one HNCMARKER, wherein the presence of said alteration indicates said cell is sensitive to a chemotherapeutic agent.

12. A method of determining the resistance of a head and neck cancer to a chemotherapeutic agent comprising identifying a alteration in at least one HNCMARKER, wherein the absence of said alteration indicates said cell is resistant to a chemotherapeutic agent.

13. The method of claim 11, wherein said alteration is a increase or a decrease.

14. The method of claim 11, wherein said alteration is determined by detecting a mutation in a HNCMARKER.

15. The method of claim 11, wherein said alteration is determined by detecting a post-translation modification of a HNCMARKER.

16. The method of claim 15, wherein said post-translational modification is selected from the group consisting of phosphorylation, ubiquitination, sumo-ylation, acetylation, alkylation, methylation, glycylation, glycosylation, isoprenylation, lipoylation, phosphopantetheinylation, sulfation, selenation and C-terminal amidation.

17. The method of claim 1, 2 or 3 wherein said treatment regimen is immunotherapy, induction chemotherapy, concurrent chemoradiotherapy or a combination thereof.

18. The method any one of claims 17, wherein said chemotherapy or chemoradiotherapy comprises is carboplatin, or one of the related class of platinum drugs, taxane, or one of the class of taxanes, or both

19. The method of claim 17, wherein said immunotherapy is Cetuximab.

20. An algorithm that is derived from the list of biomarkers in Table 1 and Table 2 which specifies how the biomarkers are associated in relation to the other biomarkers in the panel, such that the biomarker algorithm indicates a predictive or prognostic value in treatment response of head and neck cancer
